(19) United States
(12) Patent Application Publication
(10) Pub. No.: US 2025/0188083 A1
(43) Pub. Date: Jun. 12, 2025

(54) Modulators of Histone Acetyltransferase 1 and Methods of Treatment Thereof

(71) Applicant: The Board of Trustees of the Leland Stanford Junior University, Stanford, CA (US)

(72) Inventors: Joshua James Gruber, Palo Alto, CA (US); Andrew Lipchik, Palo Alto, CA (US); Michael P. Snyder, Stanford, CA (US); Steven R. Schow, Redwood Shores, CA (US)

(73) Assignee: The Board of Trustees of the Leland Stanford Junior University, Stanford, CA (US)

(21) Appl. No.: 18/573,193

(22) PCT Filed: Jun. 27, 2022
(86) PCT No.: PCT/US2022/073191

§ 371 Date: Dec. 23, 2023

Related U.S. Application Data
(60) Provisional application No. 63/215,322, filed on Jun. 25, 2021.

(51) Int. Cl. C07D 475/14; A61K 31/525

(52) U.S. Cl. CPC C07D 475/14; A61K 31/525

(57) ABSTRACT

Small molecules compounds and methods of their synthesis are provided. Small molecules identified can inhibit histone acetyltransferase 1 activity. Formulations and medicaments are also provided that are directed to the treatment of human disorders and conditions, such as, for example, neoplasms, cancers, viral, fungal, and parasitic infections, and aging. Therapeutics are also provided containing a therapeutically effective dose of one or more small molecule compounds, present either as pharmaceutically effective salt or in pure form, including, but not limited to, formulations for oral, intravenous, or intramuscular administration.

### CROSS REFERENCE TO RELATED APPLICATIONS

This application claims priority to U.S. Provisional Application Ser. No. 63/215,322 entitled “Modulators of Histone Acetyltransferase 1 and Methods of Treatment Thereof,” filed Jun. 25, 2021, which is herein incorporated by reference in its entirety.

### STATEMENT REGARDING FEDERALLY SPONSORED RESEARCH OR DEVELOPMENT

This invention was made with Government support under contract CA245024 awarded by the National Institutes of Health. The Government has certain rights in the invention.

### TECHNICAL FIELD

The disclosure is generally directed to molecules that modify histone acetyltransferase 1 (HAT1) activity and methods of treatments thereof.

### BACKGROUND

Acetyltransferases are enzymes that transfer an acetyl functional group from a donor molecule onto a biomolecule. Acetyltransferase enzymes include (but are not limited to) histone acetyltransferases, choline acetyltransferase, chloramphenicol acetyltransferase, serotonin N-acetyltransferase, and N-terminal acetyltransferases.

A common acetylation reaction is performed by the class of histone acetyltransferases (HATs), which transfer an acetyl group from acetyl CoA onto histones, which are a class of proteins that help facilitate DNA chromatin formation and regulation of gene expression. Four histones (H2A, H2B, H3 and H4) are combined to form a histone core. Each of these four histone proteins can be acetylated.

Acetylation of histones occurs on particular lysines of the protein. This reaction is portrayed in FIG. 1. The acetyl group of acetyl-CoA is transferred onto the amino group of lysine. Histone deacetylases (HDACs) remove acetyl groups from histones and back onto acetyl CoA. HAT and HDAC enzymatic activity results in particular acetylation patterns, conferring gene expression regulation. Generally, histone cores with high acetylation yields higher levels of gene expression and vice versa. Histone acetyltransferase 1 (HAT1) is an enzyme that acetylates the histone H4, and particularly on lysines 5 and 12. However, the role of di-acetylation of H4 remains enigmatic.

HAT1 is involved in a number of human disorders and conditions, including cancer, aging, immune diseases, organ rejection, and viral, fungal, and parasitic infections. HAT1 has been shown to be overexpressed in a number of cancers and to promote tumorigenesis. Further, HAT1 has been shown to promote human immunodeficiency virus (HIV) and hepatitis viruses (especially HBV) infection and replication. HAT1 is has also been shown to promote aging.

### SUMMARY OF THE DISCLOSURE

Various embodiments are directed to small molecules, methods of synthesis, medicaments formed from these small molecules, and methods for the treatment of disorders using such therapeutics. Several embodiments are directed towards compounds having an isoalloxazine core. In some embodiments, a compound of formula includes a ribityl sidechain that has been modified. In some embodiments, formulations and medicaments are provided that are directed to the treatment of disorders and/or conditions. In various embodiments of formulations and medicaments, the compounds are utilized to treat a mammalian disorder or condition. In various embodiments, the mammalian disorder or condition to be treated is a cancer, aging, immune disease, organ rejection, a viral, fungal, and/or parasitic infection.

### BRIEF DESCRIPTION OF THE DRAWINGS

The description and claims will be more fully understood with reference to the following figures and data graphs, which are presented as exemplary embodiments of the disclosure and should not be construed as a complete recitation of the scope of the disclosure.

FIG. 1 provides a reaction schematic of histone acetyltransferases.

FIGS. 2 and 3 provide a molecular structure diagram of a number of small molecule compounds in accordance with various embodiments.

FIG. 4 provides a molecular structure diagram of compound JG-2016 in accordance with various embodiments.

FIGS. 5A and 5B provide fluorescent images of organs harvested from mice injected JG-2016, generated in accordance with various embodiments.

FIG. 6 provides a schematic of a histone acetyltransferase 1 (HAT1) acetylation assay in accordance with various embodiments. HAT1/Rbap46 purified enzyme complex is incubated with 4-pentynoyl-CoA and histone H4 N-terminal biotinylated peptide in the presence or absence of test compounds. Then reaction products are bound to neutravidin 96-well plate, washed, functionalized by copper(I)-catalyzed cycloaddition click chemistry with biotin-azide. Then, streptavidin-HRP is bound and detected by amplex red fluorescence reaction.

FIG. 7 provides data graphs depicting optimization of the HAT1 acetylation assay, generated in accordance with various embodiments. Left panel: 293T cells were transfected with Flag-HAT1 (F-HAT1) alone or F-HAT1+Rbap46 plasmids, or untransfected (control) then proteins were purified by FLAG immunoprecipitation and HAT1 acetylation assays were performed. Middle panel: HAT1 acetylation reactions were performed with 0.1 M EDTA, 0.1 M DTT or both EDTA+DTT. Right panel: HAT1 acetylation reactions were performed in the presence or absence of Lysyl-CoA (10 μM) over a time course of 1-4 hours.

FIG. 8 provides a fluorescence standard curve utilized in accordance with various embodiments. The curve was generated by mixing biotinylated positive control peptide (H4 N-terminal peptide with propargylglycine residues substituted for lysines 5 and 12) with the unmodified histone H4 N-terminal peptide of equal length, followed by binding products to neutravidin plate, click chemistry addition of biotin-azide, streptavidin-HRP binding and amplex red detection. Black line indicates nonlinear curve fit through all data. Blue dashed line is a linear fit for data from 0-50% positive control (R²=0.98). Limit of detection (LoD) and limit of quantitation (LoQ) are mean±SD for three independent replicates.

FIG. 9 provides data graphs characterizing the HAT1 acetylation assay, generated in accordance with various embodiments. Left panel: High-throughput characterization of technical replicates of 100%, 50% or 0% propargylglycine containing H4 peptides mixed with unmodified peptides spotted in checkboard pattern in 96-well neutravidin-plates. Z′ score and signal window are calculated for the difference between 0-100% (top) and 0-50% (below) peptide mixtures. Right panel: High-throughput characterization of biological replicates of HAT1 acetylation reactions treated with either DMSO control or positive control bisubstrate inhibitor H4K12-CoA (10 μM).

FIG. 10 provides a dose-response curve of the HAT1 acetylation assay treated with H4K12-CoA, generated in accordance with various embodiments. IC₅₀ was calculated by 3 parameter least-squares regression assuming Hill slope −1.0.

FIG. 11 provides a schematic of virtual docking workflow used to pre-select small molecules capable of binding the acetyl-CoA cofactor binding site (highlighted) of the HAT1 crystal structure 2P0W in accordance with various embodiments. The entire NCI open chemical library was screened with Schrodinger Glide with SP and XP modes to select the top 0.001% of compounds predicted to bind. Compounds were obtained from the NCI/DTP repository.

FIG. 12 provides screening results of 37 compounds (left panel) and a dose response of best hit NSC-42186 (right panel) in accordance with various embodiments. Left panel: thirty-seven compounds from the NCI/DTP repository screened in the HAT1 acetylation assay. Inset is structure of the best hit NSC-42186. Positions around the isoalloxazine core are numbered for further reference throughout the text. Right panel: dose-response curve of NSC-42186 treatment in the HAT1 acetylation assay. IC₅₀ was calculated by 3 parameter least-squares regression assuming Hill slope −1.0.

FIG. 13 provides chemical structures (left panel) of riboflavin analogs and derivatives obtained and tested and dose-response curves (right panel) of the riboflavin analogs in tested in the HAT1 acetylation assay in accordance with various embodiments.

FIG. 14 provides screening results of compounds with structural similarity of the isoalloxazine core in accordance with various embodiments. Compounds were derived from the NCI open compounds directory, obtained from NCI/DTP, and tested for inhibitory properties in the HAT1 acetylation assay. Compounds were screened in duplicate on separate days a mean % inhibition values are plotted.

FIG. 15 provides chemical structures of compounds of top hits of the screening assay performed as described in regards to FIG. 14 in accordance with various embodiments.

FIG. 16 provides screening results of a focused library of 54 compounds containing the isoalloxazine core, as well as related structures in accordance with various embodiments. Compounds were screened in duplicate on separate days and mean % inhibition values are plotted.

FIG. 17 provides chemical structures of compounds of top hits of the screening assay performed as described in regards to FIG. 16 in accordance with various embodiments.

FIG. 18 provides chemical structures of compounds that were inactive in the screening assay performed as described in regards to FIG. 16 in accordance with various embodiments.

FIG. 19 provides screening results of synthesized compounds assessed in the HAT1 assay in accordance with various embodiments. Inset shows chemical structure of the core compound used for chemical library generation with R-group at the 10-amino position. Graph shows the mean % inhibition values for each compound performed in duplicate on separate days.

FIG. 20 provides chemical structures of compounds tested in the screening assay performed as described in regards to FIG. 19 in accordance with various embodiments.

FIG. 21 provides screening results of synthesized compounds assessed in the HAT1 assay in accordance with various embodiments. Data is mean of duplicate experiments performed on separate days.

FIGS. 22A and 22B provide chemical structures of compounds tested in the screening assay performed as described in regards to FIG. 21 in accordance with various embodiments.

FIG. 23 provides dose-response curve of JG-2016 in the HAT1 acetylation assay (left panels) and IC₅₀s of enzymatic assays assessing seven different acetyltransferases (right panel) in the presence of JG-2016 in accordance with various embodiments. Left panel: IC₅₀ was calculated by 3 parameter least-squares regression assuming Hill slope −1.0. Mean±SD of triplicate reactions is plotted. Inset: JG-2016 structure. Right panel: IC₅₀ was calculated by 10-point dose curve starting at 100 μM, followed by 3-fold serial dilutions.

FIG. 24 provides a standard curve (left panel) of positive control H4 peptides in the presence of JG-2016 (100 μM) or DMSO control and a dose response curve (right panel) of compound JG-2016 in a HAT1 acetylation assay using acetyl-CoA as the co-factor instead of 4P-CoA in accordance with various embodiments. Left panel: standard curves of positive control H4 peptides were generated in the presence of JG-2016 (100 μM) or DMSO control, followed by binding to neutravidin plates, Cu(I)-catalyzed azide-alkyne cycloaddition of biotin, then streptavidin-HRP binding and amplex red reaction and fluorescence detection (mean □ SD of duplicate samples). R2 by linear regression. Right panel: IC₅₀ was calculated by 3 parameter least-squares regression assuming Hill slope −1.0. Mean±SEM of duplicate reactions is plotted.

FIG. 25 provides fluorescence emission scans (λ=500-600 nm) of mixtures of HAT1 enzyme complex and JG-2016 (100 μM to 97.5 nM by 2-fold serial dilutions) after exposure to 472 nm light to excite the isoalloxazine ring of JG-2016 in accordance with various embodiments.

FIG. 26 provides calculation of binding constant (K_{D}) for JG-2016 to the HAT1 complex using fluorescence titration measurements (emission at λ=525 nm) of HAT1 complex (1.5 μM) with 10-point dose curve of JG-2016 (100 μM to 194 nM, 2-fold serial dilutions) in accordance with various embodiments. Data is mean+/−SD of 3 independent experiments.

FIG. 27 provides fluorescence emission values at λ=525 nM of JG-2016+buffer, JG-2016+HAT1, or HAT1 alone at indicated concentrations of JG-2016 after excitation at 472 nm in accordance with various embodiments. Legend indicates concentration of JG-2016.

FIG. 28 provides dose-response curves of JG-2016, riboflavin analogs or H4K12-CoA bisubstrate inhibitor in the HCC1806 cell line (left panel), the HCC1937 cell lines (middle panel), or the A549 cell line in accordance with various embodiments. EC₅₀ of JG-2016 was calculated for each cell line.

FIGS. 29A to 29F provide cell growth inhibition assays of 7-chloro-, 8-ethyl-isoalloxazine core library compounds and related molecules in accordance with various embodiments. FIGS. 29A to 29E: HC 806 cell line was treated with indicated compounds for 48 hours and cell density was measured by CellTiter Blue (fluorescence). FIG. 29F: HCC 806 cell IC50 is plotted against corresponding HAT1 enzymatic assay results (% inhibition) at single-dose point (100 μM).

FIGS. 30A and 30B provide blots depicting acetylation in hTert-HME1 cells were treated with indicated concentrations of JG-2016 (FIG. 30A) or in hTert-HME1 cells were starved of EGF for 16 hours, then pre-treated with JG-2016 for 30 minutes followed by stimulation with EGF for 8, 10, or 12 hours (FIG. 30B), generated in accordance with various embodiments.

FIG. 31 provides data chart showing the inhibition of tumor growth in mice treated with short-hairpin RNA of HAT1, generated in accordance with various embodiments. A549 cells were treated with either control lentivirus shRNA or 3 separate lentiviral shRNAs targeting the HAT1 mRNA, then injected into bilateral flanks of NSG mice. N=3 control shRNA mice and 7 HAT1 shRNA mice. * indicates p<0.0001 by least squares regression and extra sum-of-squares F-test.

FIG. 32 provides data chart showing the inhibition of tumor growth in mice treated with JG2016, generated in accordance with various embodiments. A549 cells were implanted into bilateral flanks of NSG mice, then vehicle control or JG-2016 at doses indicated were injected intraperitoneally every third day starting on day 13 (indicated by arrows). P-value calculated by least squares regression and extra sum-of-squares F-test. N=5 mice per group.

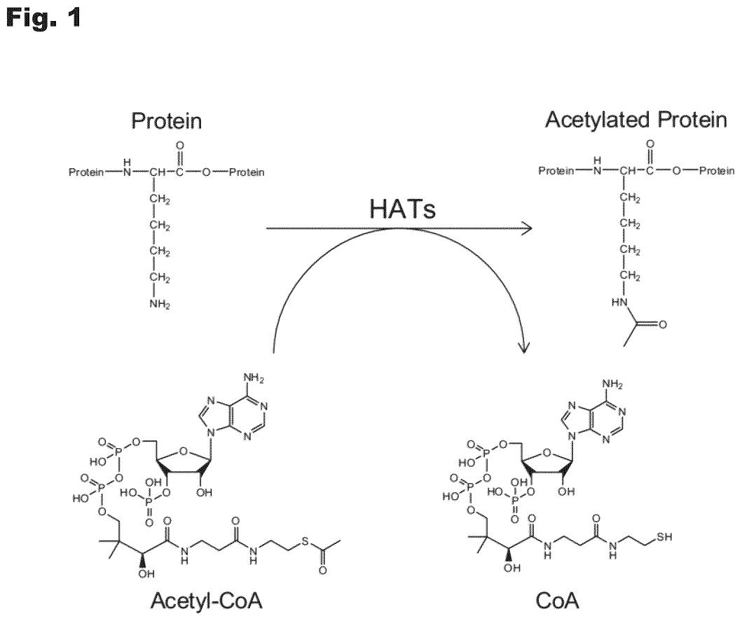

### DETAILED DESCRIPTION

Turning now to the drawings and data, molecules capable of treating disorders and/or conditions, including neoplasms and cancer, aging, immune disease, organ rejection, and viral, fungal, and/or parasitic infections, medicaments formed from these molecules, and methods for the treatment of disorders using such therapeutics are disclosed. In some embodiments, a compound of formula includes an isoalloxazine core. In some embodiments, a compound of formula includes a ribityl sidechain, which can be further modified. In some embodiments, formulations and medicaments are provided that are directed to the treatment of disorders and/or conditions. In some such embodiments these formulations and medicaments target cancers, such as, for example, leukemia, prostate, colon, lung, pancreatic and breast cancer, and potentially other disorders, including metabolic disorders or disorders where oncogenic Ras or PI 3-kinase mutations or PTEN loss are associated with the neoplastic cells. Therapeutic embodiments contain a therapeutically effective dose of one or more small molecule compounds. Embodiments allow for various formulations, including, but not limited to, formulations for oral, intravenous, or intramuscular administration. Other additional embodiments provide treatment regimens for disorders using therapeutic amounts of the small molecules.

In addition to embodiments of medicaments and treatments, embodiments are directed to the ability to visualize compounds having an isoalloxazine core via fluorescence. In some embodiments, a cell or tissue is exposed to UV light and a compound with an isoalloxazine core emits a detectable yellow-green light. Accordingly, in some embodiments, compounds having an isoalloxazine core can be monitored in a clinical and/or laboratory setting.

### Terms of Art

“Acyl” means a —C(═O)R group.

“Alcohol” means a hydrocarbon with an —OH group (ROH).

“Alkyl” refers to the partial structure that remains when a hydrogen atom is removed from an alkane.

“Alkyl phosphonate” means an acyl group bonded to a phosphate, RCO₂PO₃².

“Alkane” means a compound of carbon and hydrogen that contains only single bonds.

“Alkene” refers to an unsaturated hydrocarbon that contains at least one carbon-carbon double bond.

“Alkyne” refers to an unsaturated hydrocarbon that contains at least one carbon-carbon triple bond.

“Alkoxy” refers to a portion of a molecular structure featuring an alkyl group bonded to an oxygen atom.

“Aryl” refers to any functional group or substituent derived from an aromatic ring.

“Amine” molecules are compounds containing one or more organic substituents bonded to a nitrogen atom, RNH₂, R₂NH, or R₃N.

“Amino acid” refers to a difunctional compound with an amino group on the carbon atom next to the carboxyl group, RCH(NH₂)CO₂H.

“Azide” refers to N₃.

“Cyanide” refers to CN.

“Halogen” or “halo” means fluoro (F), chloro (CI), bromo (Br), or iodo (I).

“R” in the molecular formulas above and throughout are meant to indicate any suitable organic functionality.

### Agonists of HAT1

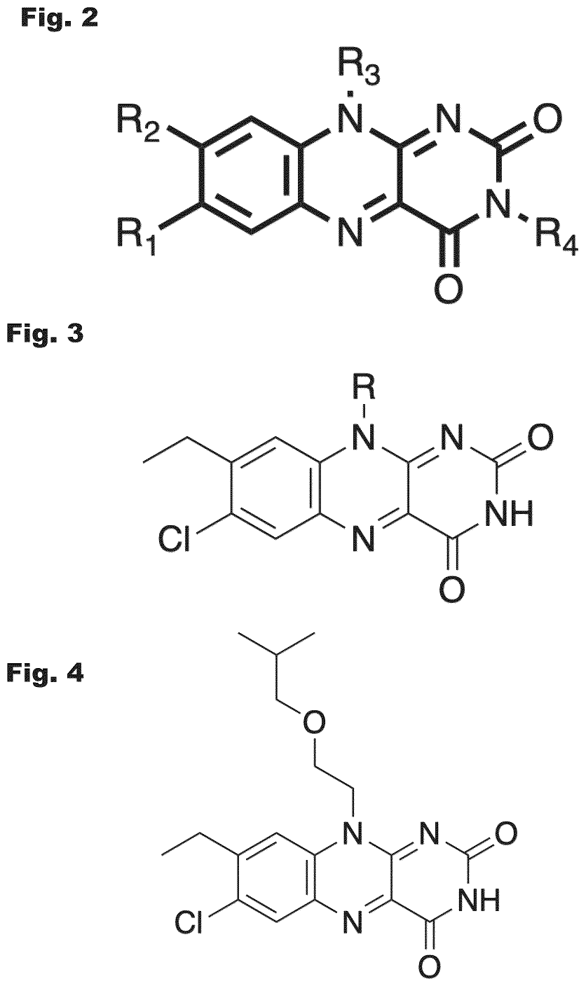

Compounds in accordance with embodiments of the disclosure are based on the discovery of various molecules that agonize HAT1. Several compounds were discovered utilizing a HAT1 activity assay, which is described within the international publication WO/2020/219598, the disclosure of which is incorporated herein by reference. Numerous compounds identified with high agonistic ability include riboflavin derivative compounds. In some embodiments, a compound of formula includes an isoalloxazine core. In some embodiments, a compound of formula includes a ribityl sidechain, which can be further modified. A chemical compound in accordance with embodiments of the invention is illustrated in FIG. 2 and pictured below. Embodiments comprise the molecules as illustrated in FIG. 2, including a compound with an isoalloxazine core and its salt of a suitable acid:

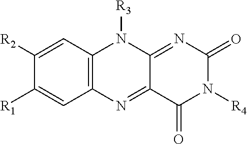

R₁ is a functional group selected from H, OH, methyl (CH₃), ethyl (C₂H₅), ethyleny (C₂H₃), ethynyl (C₂H), halogen, alkyl, alkoxy, azide (N₃), ether, NO₂, or cyanide (CN).

R₂ is a functional group selected from H, OH, methyl (CH₃), ethyl (C₂H₅), ethyleny (C₂H₃), ethynyl (C₂H), halogen, alkyl, alkoxy, azide (N₃), ether, NO₂, or cyanide (CN).

R₃ is a functional group selected from H, glucose, ribose, an alkyl chain, an alkoxy chain, a ribityl chain, OH, (CH₂)ₙOC═OR₁′, (CH₂)ₙNR₂′R₃′, (CH₂)ₙOH, (CHOH)ₙ-alkyl, (CHOH)ₙ-alkyne, (CHOH)ₙ-aryl, (CH₂)ₙOR₁′, C(OC═OR₁′)ₙ, where R₁′ is an alkyl, aryl and R₂′ and R₃′ are each independently an alkyl, alkene, alkyne, aryl, (CH₂)ₙOH, CHOH-alkyl, CHOH-alkyne, or CHOH-aryl.

R₄ is a functional group selected from H, OH, methyl, (CH₃), ethyl (C₂H₆), alkyl (including cyclic alkyls), alkoxy, NO₂, benzyl, and (CH₂) n-benzyl.

n is an independently selected integer selected from 1, 2, 3, or 4.

In certain embodiments, R₁ is a C₁ to C₃ alkyl and R₂ is a halogen. In certain embodiments, R₁ is Et or Me and R₂ is F, Cl, or Br. In certain embodiments, R₁ is Et and R₂ is C₁.

In certain embodiments, R₂ is a C₁ to C₃ alkyl and R₁ is a halogen. In certain embodiments, R₂ is Et or Me and R₁ is F, Cl, or Br. In certain embodiments, R₂ is Et and R₁ is C₁.

In certain embodiments, R₁ and R₂ are each independently a C₁ to C₃ alkyl. In certain embodiments, R₁ and R₂ are each independently Et or Me. In certain embodiments, R₁ and R₂ are each independently Et. In certain embodiments, R₁ and R₂ are each independently Me.

In certain embodiments, R₁ and R₂ are each independently a halogen. In certain embodiments, R₁ and R₂ are each independently F, Cl, or Br. In certain embodiments, R₁ and R₂ are each independently C₁.

In certain embodiments, R₁ is a C₁ to C₃ alkyl, R₂ is a halogen, and R₃ is ribose, an alkyl chain, an alkoxy chain, or a ribityl chain. In certain embodiments, R₁ is Et or Me, R₂ is F, Cl, or Br, and R₃ is ribose, an alkyl chain, an alkoxy chain, or a ribityl chain. In certain embodiments, R₁ is Et, R₂ is Cl, and R₃ is ribose, an alkyl chain, an alkoxy chain, or a ribityl chain.

In certain embodiments, R₂ is a C₁ to C₃ alkyl, R₁ is a halogen, and R₃ is ribose, an alkyl chain, an alkoxy chain, or a ribityl chain. In certain embodiments, R₂ is Et or Me, R₁ is F, Cl, or Br, and R₃ is ribose, an alkyl chain, an alkoxy chain, or a ribityl chain. In certain embodiments, R₂ is Et, R₁ is Cl, and R₃ is ribose, an alkyl chain, an alkoxy chain, or a ribityl chain.

In certain embodiments, R₁ and R₂ are each independently a C₁ to C₃ alkyl and R₃ is ribose, an alkyl chain, an alkoxy chain, or a ribityl chain. In certain embodiments, R₁ and R₂ are each independently Et or Me and R₃ is ribose, an alkyl chain, an alkoxy chain, or a ribityl chain. In certain embodiments, R₁ and R₂ are each independently Et and R₃ is ribose, an alkyl chain, an alkoxy chain, or a ribityl chain. In certain embodiments, R₁ and R₂ are each independently Me and R₃ is ribose, an alkyl chain, an alkoxy chain, or a ribityl chain.

In certain embodiments, R₁ and R₂ are each independently a halogen and R₃ is ribose, an alkyl chain, an alkoxy chain, or a ribityl chain. In certain embodiments, R₁ and R₂ are each independently F, Cl, or Br and R₃ is ribose, an alkyl chain, an alkoxy chain, or a ribityl chain. In certain embodiments, R₁ and R₂ are each independently C₁ and R₃ is ribose, an alkyl chain, an alkoxy chain, or a ribityl chain.

In certain embodiments, R₁ is a C₁ to C₃ alkyl, R₂ is a halogen, and R₃ is ribose,

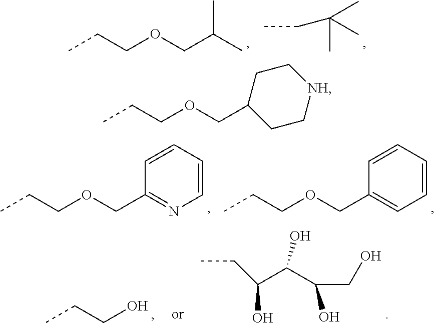

In certain embodiments, R₁ is Et or Me, R₂ is F, Cl, or Br, and R₃ is ribose,

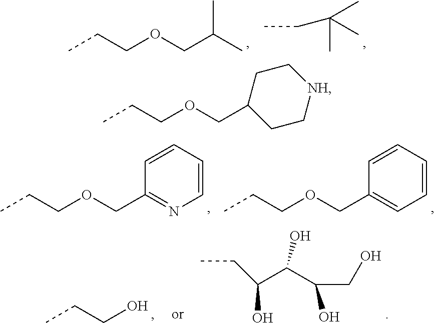

or In certain embodiments, R₁ is Et, R₂ is Cl, and R₃ is ribose,

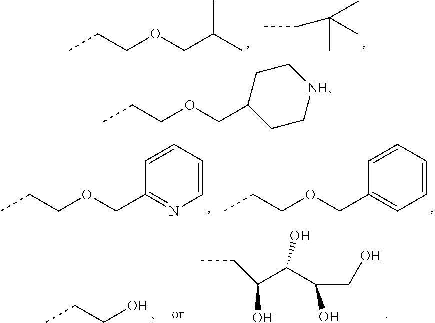

In certain embodiments, R₂ is a C₁ to C₃ alkyl, R₁ is a halogen, and R₃ is ribose,

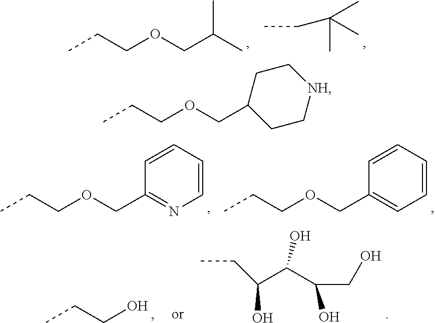

In certain embodiments, R₂ is Et or Me, R₁ is F, Cl, or Br, and R₃ is ribose,

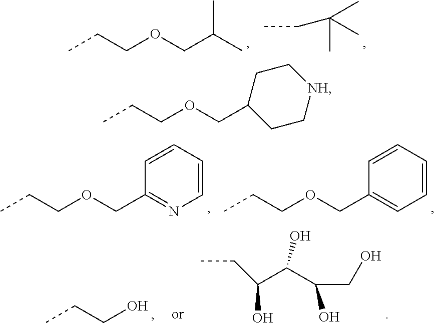

In certain embodiments, R₂ is Et, R₁ is Cl, and R₃ is ribose,

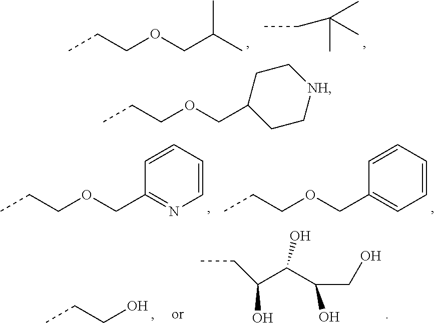

In certain embodiments, R₁ and R₂ are each independently a C₁ to C₃ alkyl and R₃ is ribose,

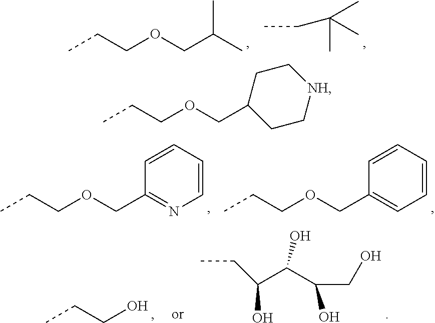

In certain embodiments, R₁ and R₂ are each independently Et or Me and R₃ ribose,

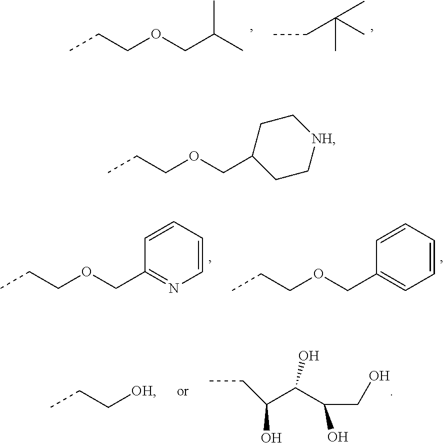

In certain embodiments, R₁ and R₂ are each independently Et and R₃ ribose,

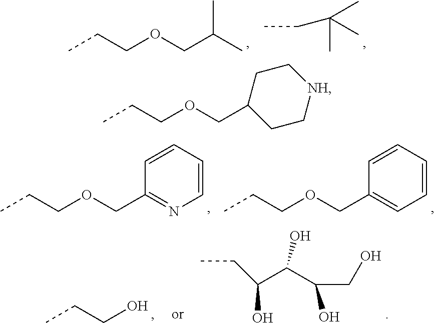

In certain embodiments, R₁ and R₂ are each independently Me and R₃ is ribose,

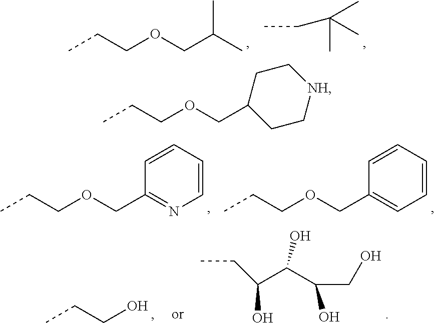

In certain embodiments, R₁ and R₂ are each independently a halogen and R₃ is ribose,

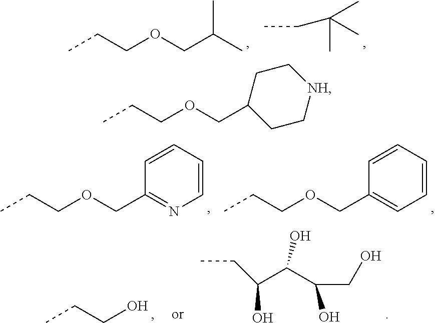

In certain embodiments, R₁ and R₂ are each independently F, Cl, or Br and R₃ is ribose,

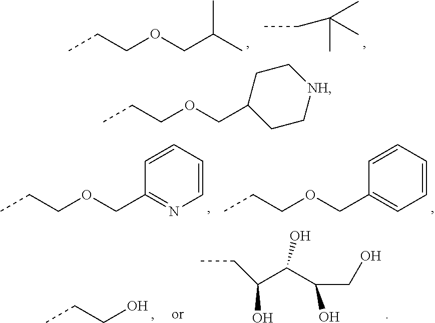

In certain embodiments, R₁ and R₂ are each independently Cl and R₃ is ribose,

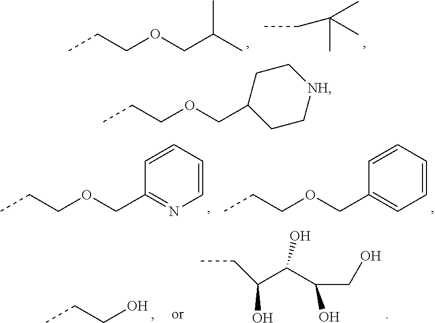

Further examples of compounds are provided in the attached exemplary data, including a number of molecules with an isoalloxazine core with various functional groups. Exemplary compounds with an isoalloxazine core include NSC-42186, riboflavin tetrabutyrate, NSC-3064, and NSC-275266). Other compounds were found to have inhibitory activity include NSC-105827, NSC-156563, NSC-332670, and NSC-83950 (see attached exemplary data).

A chemical compound with an isoalloxazine core in accordance with embodiments of the invention is illustrated in FIG. 3 and pictured below. Embodiments comprise the molecules as illustrated in FIG. 3, including a compound with an isoalloxazine core and its salt of a suitable acid:

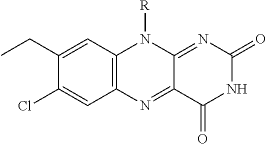

R is a functional group selected from H, glucose, ribose, an alkyl chain, an alkoxy chain, a ribityl chain, OH, (CH₂)ₙOC═OR₁′, (CH₂)ₙNR₂′R₃′, (CH₂)ₙOH, (CHOH)ₙ-alkyl, (CHOH)ₙ-alkyne, (CHOH)ₙ-aryl, (CH₂)ₙOR₁′, C(OC═OR₁′)ₙ, where R₁′ is an alkyl, alkene, alkyne, aryl and R₂′ and R₃′ are each independently an alkyl, alkene, alkyne, aryl, (CH₂)ₙOH, CHOH-alkyl, CHOH-alkyne, or CHOH-aryl.

n is an independently selected integer selected from 1, 2, 3, or 4.

In certain embodiments, R is an alkyl chain, an alkoxy chain, or a ribityl chain. In certain embodiments, R is ribose,

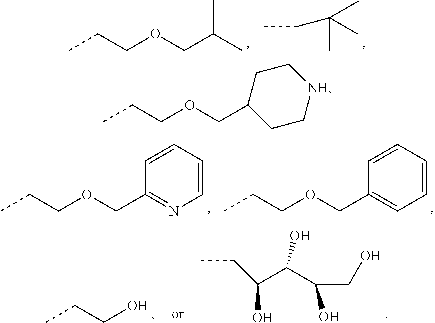

Further examples of compounds are provided in the attached exemplary data, including a number of molecules with an isoalloxazine core with various functional groups. Exemplary compounds with an isoalloxazine core include JG-2016, which includes a 1-ethoxy-2-methyl-propane sidechain at the amino-10 position of the isoalloxazine core and is illustrated in FIG. 4 and pictured below:

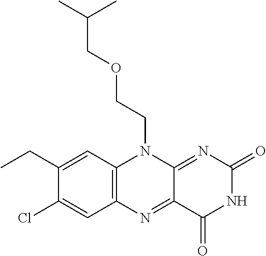 [SMILES: CCc1cc2c(cc1Cl)nc1c(=O)[nH]c(=O)nc-1n2CCOCC(C)C]

It will be understood that compounds in this invention may exist as stereoisomers, including enantiomers, diastereomers, cis, trans, syn, anti, solvates (including hydrates), tautomers, and mixtures thereof, are contemplated in the compounds of the present disclosure.

The compounds can also be related to pharmaceutically acceptable salts, polymorphs, co-crystals, or depot formulations. A “pharmaceutically acceptable salt” retains the desirable biological activity of the compound without undesired toxicological effects. Salts can be salts with a suitable acid, including, but not limited to, hydrochloric acid, hydrobromic acid, sulfuric acid, phosphoric acid, nitric acid and the like; acetic acid, oxalic acid, tartaric acid, succinic acid, malic acid, benzoic acid, pamoic acid, alginic acid, methanesulfonic acid, naphthalenesulphonic acid, and the like. Also, incorporated cations can include ammonium, sodium, potassium, lithium, zinc, copper, barium, bismuth, calcium, and the like; or organic cations such as tetraalkylammonium and trialkylammonium cations. Also useful are combinations of acidic and cationic salts. Included are salts of other acids and/or cations, such as salts with trifluoroacetic acid, chloroacetic acid, and trichloroacetic acid.

Other compounds with an isoalloxazine core, as well as modified isoalloxazine core molecules, suitable for practice of the present invention will be apparent to the skilled practitioner. Some molecules may include any diastereomeric isoalloxazine core compound. Furthermore, these molecules may employ several mechanisms of action to inhibit HAT1 activity, even if the molecules are not structurally identical to the compounds shown above.

### Modes of Treatment

In some embodiments, the compounds described herein, especially compounds with an isoalloxazine core are administered in a therapeutically effective amount as part of a course of treatment. As used in this context, to “treat” means to ameliorate at least one symptom of the disorder to be treated or to provide a beneficial physiological effect. For example, one such amelioration of a symptom could be inhibition of neoplastic proliferation. Assessment of neoplastic proliferation can be performed in many ways, including, but not limited to assessing changes in tumor diameter, changes in tumor bioluminescence, changes in tumor volume, changes in tumor mass, or changes in neoplastic cell proliferation rate. Subjects to be treated include (but are not limited to) animals, mammals, pets, livestock, zoo animals, laboratory research animals, and humans.

In some embodiments, an individual to be treated has been diagnosed as having a neoplastic growth or cancer. In many embodiments, the neoplasm is characterized as fast-growing, aggressive, malignant, HER2-mutant or amplified, EGF/EGFR-mutant or amplified or positive, Ras-mutant, PTEN-negative, having PI 3-kinase mutations, benign, metastatic, or nodular. A number of cancers can be treated, including (but not limited to) acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, neuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, or vascular tumors. In some embodiments, a compound is used as a prophylaxis to mitigate and/or prevent cancer initiation and/or relapse.

In some embodiments, an individual to be treated has been diagnosed as having a viral, fungal, and/or parasitic infection. In many embodiments, the individual is infected with human immunodeficiency virus (HIV) or hepatitis B virus (HBV). In some embodiments, a compound is used as a prophylaxis to mitigate and/or prevent viral, fungal, and/or parasitic infection.

In some embodiments, an individual to be treated has disorder related to aging. In some embodiments, a compound is used as a prophylaxis to mitigate the effects of aging.

In some embodiments, an individual to be treated has an immune disease. In some embodiments, a compound is used as a prophylaxis to mitigate the effects of immune disease.

In some embodiments, an individual to be treated has had an organ transplant to mitigate organ rejection. In some embodiments, a compound is administered prior to, during, and/or after an organ transplant procedure.

A therapeutically effective amount can be an amount sufficient to mitigate, prevent, reduce, ameliorate or eliminate the symptoms of diseases or pathological conditions susceptible to such treatment, such as, for example, treatments for cancer, aging, immune disease, organ rejection, or viral, fungal, and/or parasitic infection.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to non-neoplastic cells and, thereby, reduce side effects.

Data obtained from cell culture assays or animal studies can be used in formulating a range of dosage for use in humans. If the medicament is provided systemically, the dosage of such compounds lies preferably within a range of circulating concentrations that include the ED₅₀ with little or no toxicity. The dosage may vary within this range depending upon the dosage form employed and the route of administration utilized. For any compound used in a method of the disclosure, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of neoplastic growth) as determined in cell culture. Such information can be used to more accurately determine useful doses in humans. Levels in plasma may be measured, for example, by liquid chromatography coupled to mass spectrometry. In some embodiments, a cytotoxic effect is achieved with an IC₅₀ less than 500 μM, 200 μM, 100 μM, 50 μM, 20 μM, 10 μM, or 5 μM. Dosing can also be provided per weight. Accordingly, in some embodiments, a cytotoxic effect is achieved with a dose between 1 mg/kg to 50 mg/kg one to four times daily. In various embodiments, a cytotoxic effect is achieved with a dose of about: 1 mg/kg, 2 mg/kg, 5 mg/kg, 10 mg/kg, 15 mg/kg, 20 mg/kg, 25 mg/kg, 30 mg/kg, 35 mg/kg, 40 mg/kg, 45 mg/kg, or 50 mg/kg provided one to four times daily. In some embodiments, a cytotoxic effect is achieved with a dose between 0.5 mcg to 2000 mcg one to four times daily. In various embodiments, a cytotoxic effect is achieved with a dose of about: 0.5 mcg, 1.0 mcg, 2.0 mcg, 5.0 mcg, 10 mcg, 20 mcg, 30 mcg, 40 mcg, 50 mcg, 100 mcg, 200 mcg, 300 mcg, 400 mcg, 500 mcg, 600 mcg, 700 mcg, 800 mcg, 900 mcg, 1000 mcg, 1200 mcg, 1300 mcg, 1400 mcg, 1500 mcg, 1600 mcg, 1700 mcg, 1800 mcg, 1900 mcg, 2000 mcg one to four times daily.

An “effective amount” is an amount sufficient to effect beneficial or desired results. For example, a therapeutic amount is one that achieves the desired therapeutic effect. This amount can be the same or different from a prophylactically effective amount, which is an amount to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled practitioner will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result. A single small molecule compound may be administered, or combinations of various small molecule compounds may also be administered.

In a number of embodiments, compounds described herein are administered in combination with an appropriate standard of care, such as the standard of care established by the United States Federal Drug Administration (FDA). In many embodiments, compounds are administered in combination with other cytotoxic compounds, especially FDA-approved compounds.

A number of additional or alternative treatments and medications are available to treat neoplasms and cancers, such radiotherapy, chemotherapy, immunotherapy, and hormone treatments. Classes of anti-cancer or chemotherapeutic agents can include alkylating agents, platinum agents, taxanes, vinca agents, anti-estrogen drugs, aromatase inhibitors, ovarian suppression agents, endocrine/hormonal agents, bisphosphonate therapy agents and targeted biological therapy agents. Medications include (but are not limited to) cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolomide, topotecan, vincristine, vinblastine, eribulin, mutamycin, capecitabine, capecitabine, anastrozole, exemestane, letrozole, leuprolide, abarelix, buserelin, goserelin, megestrol acetate, risedronate, pamidronate, ibandronate, alendronate, zoledronate, and tykerb. Accordingly, an individual may be treated, in accordance with various embodiments, by a single medication or a combination of medications described herein. For example, common treatment combination is cyclophosphamide, methotrexate, and 5-fluorouracil (CMF). Furthermore, several embodiments of treatments further incorporate immunotherapeutics, including denosumab, bevacizumab, cetuximab, trastuzumab, pertuzumab, alemtuzumab, ipilimumab, nivolumab, ofatumumab, panitumumab, and rituximab.

Dosing and therapeutic regimens can be administered appropriate to the neoplasm to be treated, as understood by those skilled in the art. For example, 5-FU can be administered intravenously at dosages between 25 mg/m² and 1000 mg/m². Methotrexate can be administered intravenously at dosages between 1 mg/m² and 500 mg/m².

It is also possible to add agents that improve the solubility of these compounds. For example, the claimed compounds can be formulated with one or more adjuvants and/or pharmaceutically acceptable carriers according to the selected route of administration. For oral applications, gelatin, flavoring agents, or coating material can be added. In general, for solutions or emulsions, carriers may include aqueous or alcoholic/aqueous solutions, emulsions or suspensions, including saline and buffered media. Parenteral vehicles can include sodium chloride and potassium chloride, among others. In addition, intravenous vehicles can include fluid and nutrient replenishers, electrolyte replenishers and the like.

Numerous coating agents can be used in accordance with various embodiments of the invention. In some embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

Various embodiments of formulations also include at least one disintegrating agent, as well as diluent. In some embodiments, a disintegrating agent is a super disintegrating agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

Several embodiments of a formulation further utilize other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Multiple embodiments of a formulation also include a surfactant. In certain embodiments, surfactants are selected from the group consisting of Tween 80, sodium lauryl sulfate, and docusate sodium.

Many embodiments of a formulation further utilize a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

Various embodiments of a formulation also include a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Modes of administration, in accordance with multiple embodiments, include, but are not limited to, oral, transdermal, transmucosal (e.g., sublingual, nasal, vaginal or rectal), or parenteral (e.g., subcutaneous, intramuscular, intravenous, bolus or continuous infusion). The actual amount of drug needed will depend on factors such as the size, age and severity of disease in the afflicted individual. The actual amount of drug needed will also depend on the effective concentration ranges of the various active ingredients.

A number of embodiments of formulations include those suitable for oral administration. Formulations may conveniently be presented in unit dosage form and may be prepared by any of the methods well known in the art of pharmacy. Typically, these methods include a step of bringing into association a compound of at least one embodiment described herein, or a pharmaceutically salt, prodrug, or solvate thereof (“active ingredient”) with the carrier which constitutes one or more accessory ingredients.

Embodiments of formulations disclosed herein suitable for oral administration may be presented as discrete units such as capsules, cachets or tablets each containing a predetermined amount of the active ingredient; as a powder or granules; as a solution or a suspension in an aqueous liquid or a nonaqueous liquid; or as an oil-in-water liquid emulsion or a water-in-oil liquid emulsion. Multiple embodiments also compartmentalize various components within a capsule, cachets, or tablets, or any other appropriate distribution technique.

Several embodiments of pharmaceutical preparations include tablets, push-fit capsules made of gelatin, as well as soft, sealed capsules made of gelatin and a plasticizer, such as glycerol or sorbitol. Tablets, in a number of embodiments, may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. Push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Preservatives and other additives, like antimicrobial, antioxidant, chelating agents, and inert gases, can also be present. (See generally, Remington's Pharmaceutical Sciences, 16th Edition, Mack, (1980), the disclosure of which is incorporated herein by reference.)

### Fluorescence Monitoring

In some embodiments, the compounds described herein, especially compounds with an isoalloxazine core are visualized via fluorescence imaging. Any appropriate fluorescent imaging technique can be utilized. For example, compounds can be monitored utilizing a fluorescent technique such as optical molecular imaging and transcutaneous fluorescence spectroscopy. Accordingly, compounds with an isoalloxazine core can be monitored for their distribution, localization, bioavailability, sustainability, or any other characteristic determinable via fluorescent imaging.

To visualize compounds with an isoalloxazine core, in accordance with various embodiments, a UV excitation source can be provided. Compounds with an isoalloxazine core emit a yellow-green light upon UV excitation. The yellow-green emission can be detected by any appropriate detection system, such as those used in the laboratory or clinic.

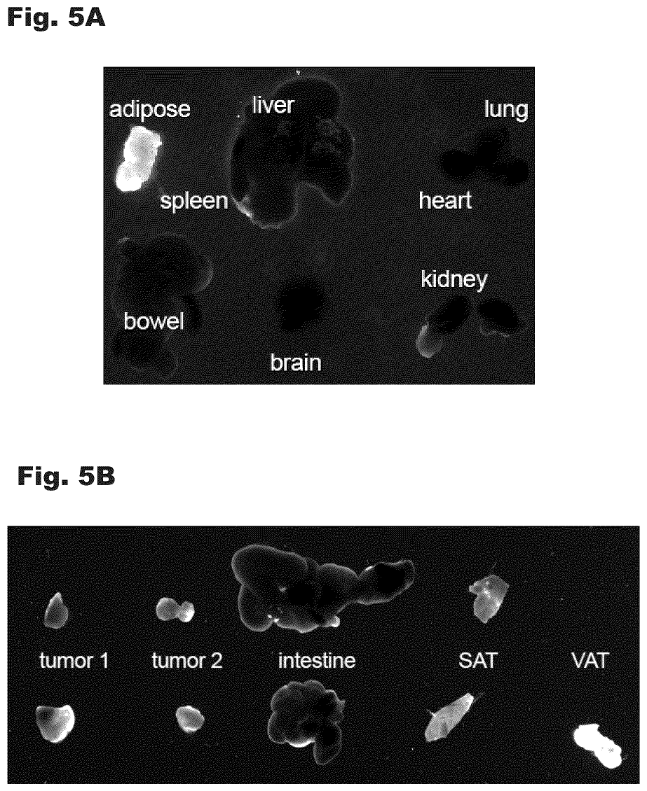

In some embodiments, compounds are monitored for their ability to infiltrate and/or surround a neoplastic growth (e.g., tumor). Provided in FIGS. 5A and 5B are examples of imaging to detect a compound with an isoalloxazine core.

Healthy mice were intraperitoneally injected with JG-2016 at 500 mg/kg and animals were sacrificed 24 hours later. Organs were harvested and excited with UV light (230 nm) and imaged with a yellow-green filter. Imaging results show compound accumulation in various tissues at various amounts, and high accumulation adipose tissue (FIG. 5A).

Mice bearing bilateral A549 tumors were treated with JG-2016 at dose of 100 mg/kg once then sacrificed after 24 hours and organs collected and imaged. Imaging shows compound accumulation in subcutaneous adipose tissues (SAT), visceral adipose tissue (VAT), and tumors (FIG. 5B).

### Exemplary Data

Biological and chemical data supports the development and use of the aforementioned compounds in a variety of embodiments, including synthesis and methods use. It is noted that embodiments of compounds, especially isoalloxazine core molecules, in accordance with the disclosure, inhibit HAT1 activity and thus can further kill and/or inhibit the growth of neoplastic cells.

### An Acetyl-Click Screening Platform Identifies Small Molecule Inhibitors of Histone Acetyltransferase 1 (HAT1)

HAT1 is a central regulator of chromatin synthesis that acetylates nascent histone H3:H4 tetramers in the cytoplasm. It may have a role in cancer metabolism by driving transport of acetyl groups from the cytoplasm, where they are produced by mitochondrial reactions, to the nucleus for consumption in epigenetic processes. This is because the HAT1 di-acetylation mark is not propagated in chromatin and instead is de-acetylated after nascent histone insertion into chromatin. Thus, HAT1 likely provides a nuclear source of free acetate that may be recycled to acetyl-CoA for nuclear acetylation reactions. Correspondingly, suppression of HAT1 protein expression impairs tumor growth. To ascertain whether targeting HAT1 is a viable anti-cancer treatment strategy, small molecule inhibitors of HAT1 were identified and tested. A high-throughput HAT1 acetyl-click assay was developed to facilitate drug discovery and enzymology. Screening of small molecules libraries led to the discovery of multiple riboflavin analogs that inhibited HAT1 enzymatic activity. Compounds were refined by synthesis and testing of over 70 analogs, which yielded structure-activity relationships. The isoalloxazine core was required for enzymatic inhibition, whereas modifications of the ribityl sidechain improved enzymatic potency and cellular growth suppression. One compound (JG-2016) that showed relative specificity towards HAT1 compared to other acetyltransferases was further characterized for biological activity. It suppressed growth of human cancer cells lines in vitro, impaired enzymatic activity in cellulo, and interfered with tumor growth in vivo. This is the first report of a small molecule inhibitor of the HAT1 enzyme complex and represents a step towards targeting this pathway for cancer therapy.

Nutrient metabolism and epigenetic reactions are integrated and sensed by cells to ensure that adequate substrates are available to meet the demands of transcriptional programs that spur cell division. By studying genes induced by epidermal growth factor (EGF) HAT1 was identified as the human acetyltransferase most highly induced by EGF stimulation in mammary cells. HAT1 was also required for rapid cell proliferation and tumor formation in vivo. These data indicate that HAT1 plays a critical role in coordinating anabolic and epigenetic processes for cell division that drives tumor growth.

HAT1 was the first histone acetyltransferase gene isolated, and subsequent work has established that it plays a critical role in chromatin replication, the process of making new nucleosomes during S-phase. In the cytosol, HAT1 di-acetylates histone H4 on lysines 5 and 12 of the amino-terminal histone tail. It then transits to the nucleus together with histone tetramers or disomes and other histone chaperones to deposit nascent histones at the replication fork, or other sites of nucleosome insertion. Then HAT1 is released from chromatin and the HAT1 di-acetylation mark on histone H₄ is quickly removed within a span of 15-30 minutes by the action of histone deacetylases. Thus, HAT1 does not directly acetylate chromatin, and the di-acetylation mark placed by HAT1 is not propagated to mature chromatin.

Prior work suggested a model whereby free acetate derived from de-acetylation of nascent histones was recycled to acetyl-CoA via acetyl-CoA-synthetases to provide substrate for nascent chromatin acetylation (J. J. Gruber, et al., Mol Cell 75:711-724 e5, 2019, the disclosure of which is incorporated herein by reference). As each nascent nucleosome of newly replicated chromatin contributes four HAT1-dependent acetyl groups, this should provide adequate acetyl-CoA to allow for histone acetylation of the much sparser promoter and enhancer sites. Indeed, histone H3 acetylation marks are reduced in cells depleted from HAT1, as expected from this model. In addition, CBP auto-acetylation is strongly dependent on HAT1 which also suggests a role for HAT1 in governing nuclear acetyl flux. Other links between mitochondrial processes and HAT1 function have also been reported.

Although these genetic studies have shed light on HAT1 function, further development of chemical probes should prove useful to distinguish the importance of the enzyme's catalytic activity from its structural role in protein:protein or protein:DNA complexes. Recently, a HAT1 bisubstrate inhibitor was designed by chemically ligating co-enzyme A to the ζ-amine of lysine 12 in the histone H4 N-terminal 20-mer peptide, yielding a Kᵢ of ˜1 nM towards bacterially-expressed recombinant HAT1 (L. Ngo, T. Brown, and TG Zheng, Chem Biol Drug Des, 2019). Although, useful for enzymatic assays, this probe is not cell permeable and therefore of limited utility to study cellular processes dependent on HAT1. Therefore, it was sought to identify and design small molecule modulators of HAT1 to probe the effects of HAT1 activity in cells and validate its role as a pro-tumorigenic factor.

The design of small molecule acetyltransferase inhibitors has been hampered by non-specific and low-throughput assays, which have tended to yield bio-reactive molecules. However, specific, potent, small molecule acetyltransferase inhibitors targeting CBP/p300 and KAT6A/B have recently been described. Previous work pioneered peptide-based sensors of non-receptor tyrosine kinases for cellular detection and monitoring of post-translational modification events including small molecule kinase inhibitor treatments (A. M. Lipchik, et al., J Am Chem Soc 137:2484-94, 2015, the disclosure of which is incorporated herein by reference). A generalizable in silico pipeline was developed to design, optimize and screen kinase-specific peptide substrates for drug discovery screens (A. M. Lipchik, et al, J Am Chem Soc 137:2484-94, 2015, the disclosure of which is incorporated herein by reference). The use of lanthanide coordination by peptide substrates allowed for the development of screening assays without the need for post-translational modification-specific antibodies. Therefore, these approaches were adapted with recent advances in acetylation monitoring to build a high-throughput, peptide-based sensor assay for HAT1 acetylation activity to facilitate drug discovery.

### Results and Data

### Design and Validation of a HAT1 High-Throughput Enzymatic Assay

HAT1 chemical probe screens or high-throughput enzymatic assays have yet to be described. Therefore, a HAT1 enzymatic assay was designed to specifically and rapidly measure the HAT1 di-acetylation product using a click-chemistry approach (FIG. 6). The click-chemistry acetyl-CoA analog 4-pentynoyl-CoA allows for enzymatic transfer of an alkyne handle via an acylation reaction. First, the HAT1 enzyme complex (HAT1+Rbap46) purified from human cells is co-incubated with 4-pentynoyl-CoA co-factor and a biotinylated H4 N-terminal peptide to allow for HAT1-dependent pentoylation of the peptide substrate at lysines 5 and 12. Then, reaction products are bound to a neutravidin capture plate, followed by Cu(I)-catalyzed alkyne-azide cycloaddition with biotin-azide. After streptavidin-HRP binding, fluorescence signal can be detected by peroxidation of amplex red.

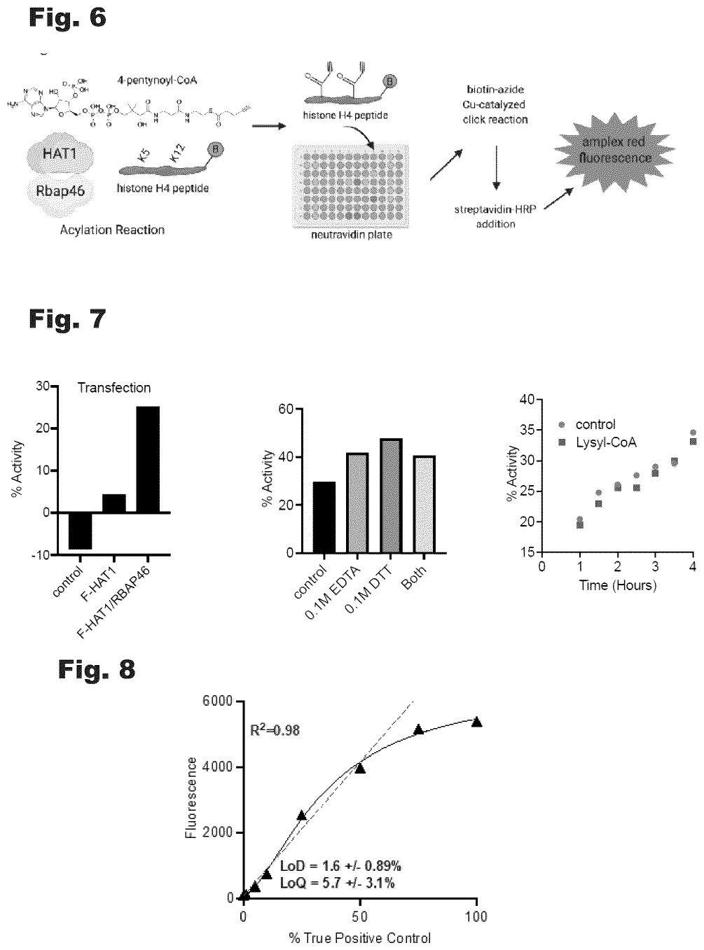

This assay was dependent on exogenous expression and co-purification of both HAT1 and Rbap46 from a human cell line (FIG. 7, left panel). In contrast, utilization of HAT1 alone was less active. Next, assay performance was optimized (FIG. 7, middle panel) and quantified. A standard curve was generated using a histone H4 peptide manufactured to have terminal alkynes at the 5^{th} and 12^{th} positions of the H4 N-terminal peptide (FIG. 8) allowing for quantification of reaction products. The assay demonstrated linear performance (R²=0.98) when up to 50% true positive control peptides were used. Therefore, assay conditions were calibrated to produce signal within this upper threshold. The limit of detection (LoD) was 1.6±0.89% defined as the percentage of positive control peptide that gave a fluorescence reading corresponding to 3× the standard deviation of the unacetylated negative control peptide above baseline. The limit of quantitation (LoQ) was 5.7±3.1% defined as the percentage of positive control peptide that gave fluorescence output corresponding to 10× the standard deviation greater than the baseline negative control. Thus the assay was designed to operate at fluorescent detection values greater than the LoQ.

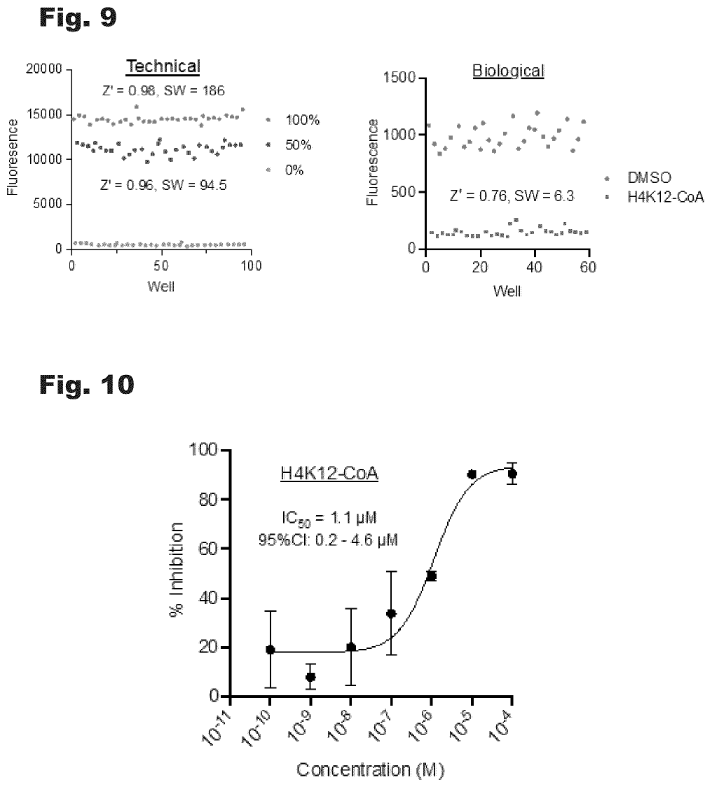

Next, high-throughput assay performance was characterized using technical controls. A 96-well plate was arrayed in checkboard configuration consisting of H4 N-terminal peptides with 0%, 50% or 100% alkyne-containing positive control peptides (FIG. 9, left panel). This allowed us to calculate the Z′ factor and the signal window (SW) for the maximal range of the assay (0-100%) and also for the linear range of the assay (0-50%). A Z′ factor between 0.5 and 1 indicates the assay performs adequately for high-throughput screening (HTS) as it indicates a robust dynamic range. Similarly, a SW >2 also indicates wide separation between positive and negative controls suitable for HTS. Both the maximal range (0-100%) and linear range (0-50%) of the assay demonstrated excellent performance characteristics (Z′>0.5, SW >2; FIG. 9, left panel). Therefore, the assay is technically suitable for HTS.

To determine if the assay maintained appropriate HTS parameters under biological conditions the HAT1/Rbap46 complex was incubated with and without the H4K12-CoA bi-substrate inhibitor (positive control inhibitor), which caused robust inhibition with suitable high-throughput performance metrics Z′ and SW (FIG. 9, right panel). To further demonstrate the biological characteristics of the assay we measured the IC₅₀ for HAT1 inhibition by the bi-substrate inhibitor. H4K12-CoA inhibited HAT1/Rbap46 activity with an IC₅₀ of ˜1 μM (FIG. 10). As this bi-substrate inhibitor has been shown to be a specific inhibitor of HAT1, but not other acetyltransferases, this validates the specificity of the HAT1 high-throughput assay. Finally, although lysyl-CoA is a validated bi-substrate inhibitor for CBP/p300, it had no inhibitory activity towards HAT1/Rbap46 in the assay (FIG. 7, right panel), thereby demonstrating assay specificity. Together these results demonstrate the ability of the HAT1/Rbap46 assay to detect biologically relevant acetylation.

Structural Modeling Coupled with Enzymatic Screening to Identify HAT1 Inhibitors

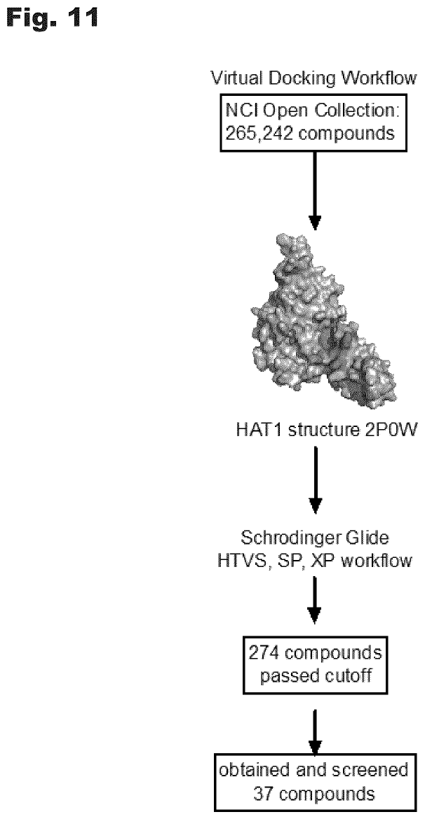

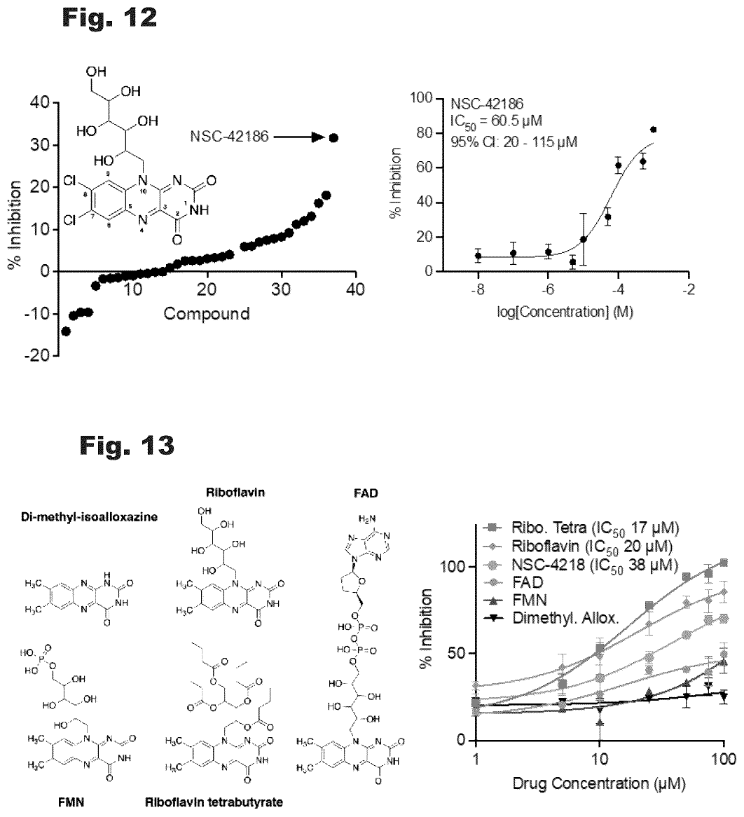

Human HAT1 has been crystalized at high-resolution (1.9-Å) revealing residues that comprise binding surfaces for the histone H4 N-terminal substrate and the acetyl-CoA cofactor. The pantotheine moiety of acetyl-CoA resides in a canyon that orients the thioester bond in close proximity to the lysine 12 side-chain of H4, which is the preferred substrate site for acetyl transfer. This structural data was used to build a virtual docking workflow to identify small molecules with appropriate physiochemical properties to occupy the cofactor binding site (FIG. 11). The NCI open collection of 265,242 molecules was screened against the HAT1 co-factor binding sites using Schrodinger Glide-based virtual screening with increasing precision cutoffs. he top 0.001% of compounds yielded 274 hits from the starting collection. Of these, 39 were obtained and screened with the HAT1 acetylation assay at a single dosage (100 μM, equivalent to the co-factor concentration). The best compound (NSC-42186) was a natural-product derivative of riboflavin that displayed 31% inhibition at 100 μM (FIG. 12, left panel). NSC-42186 showed appropriate dose-response activity with an enzymatic IC₅₀ of 60.5 μM (95% CI: 20-115 μM; FIG. 12, right panel). Thus, this combined virtual and enzymatic screening approach identified small molecule candidate HAT1 inhibitors.

NSC-42186 contains 7,8-di-chloro substitutions of the tri-cyclic isoalloxazine ring that are the sole features that distinguish it from the 7,8-di-methyl isoalloxazine of riboflavin (ring numbering scheme is shown in FIG. 12). Riboflavin, commonly known as vitamin B2, is an essential nutrient in metazoans where it is further metabolized to flavin-mononucleotide (FMN) and flavin-adenine-dinucleotide (FAD), which are cofactors for flavin-containing proteins. To determine if a flavin class effect contributed to HAT1 inhibition the enzymatic assay was repeated with other flavonoids including riboflavin, di-methyl-isoalloxazine, FMN, FAD, and riboflavin tetrabutyrate (FIG. 13). NSC-42186, riboflavin and riboflavin tetrabutyrate all inhibited HAT1 enzymatic activity with low-micromolar IC₅₀s of 38, 20, and 17 μM, respectively (FIG. 13). However, there was no detectable inhibitory activity for di-methyl-alloxazine, FMN, nor FAD. Because riboflavin, FAD and FMN have similar redox potentials it is unlikely that redox cycling is a common mechanism for HAT1 inhibition with this class of compounds. Furthermore, free radical generation by NSC-42186 was not detected under the above assay conditions. Therefore, certain riboflavin analogs, but not all, can modulate HAT1 acetylation activity and this effect appears to be independent of redox cycling.

### Screening of Focused Libraries to Develop Structure-Activity Relationships

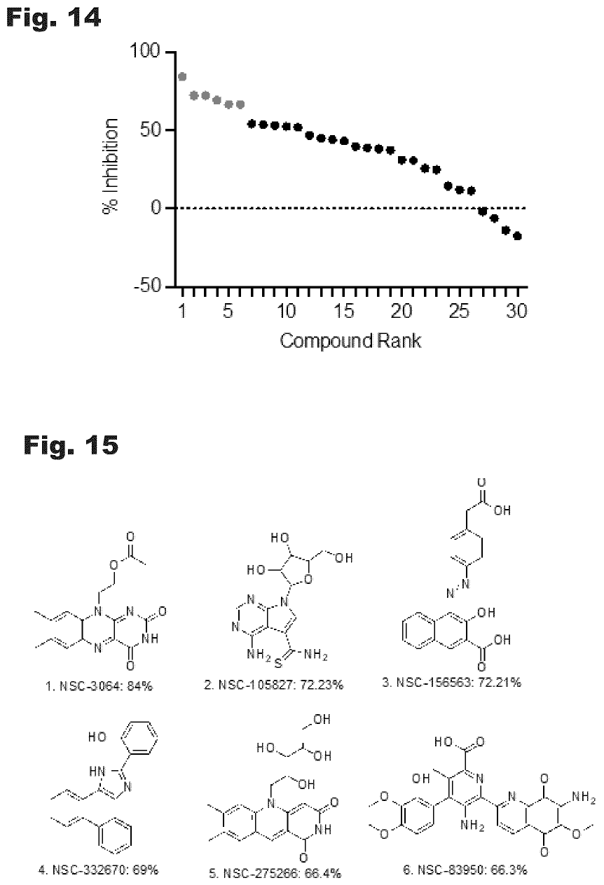

As various riboflavin-derived analogs affected HAT1 enzymatic activity, an expanded collection of similar compounds was assessed. Computational structure searches were performed to identify other compounds in the NCI open library with similarity to the isoalloxazine core of NSC-42186. Of these, 30 additional compounds were experimentally characterized with the assay (FIG. 14). The top hit (NSC-3064) also contained a 7,8-di-methyl-isoalloxazine core but with a different Ni appended sidechain comprised of an acetyl-ethyl (FIG. 15). Also, the fifth best hit (NSC-275266) also contained a 7,8-dimethyl-isoalloxazine core but lacked the amino group at position 4. Therefore, the 7,8-di-substituted-isoalloxazine structure is repeatedly found in compounds with HAT1-inhibitory activity, although the Nio-appended sidechain can vary.

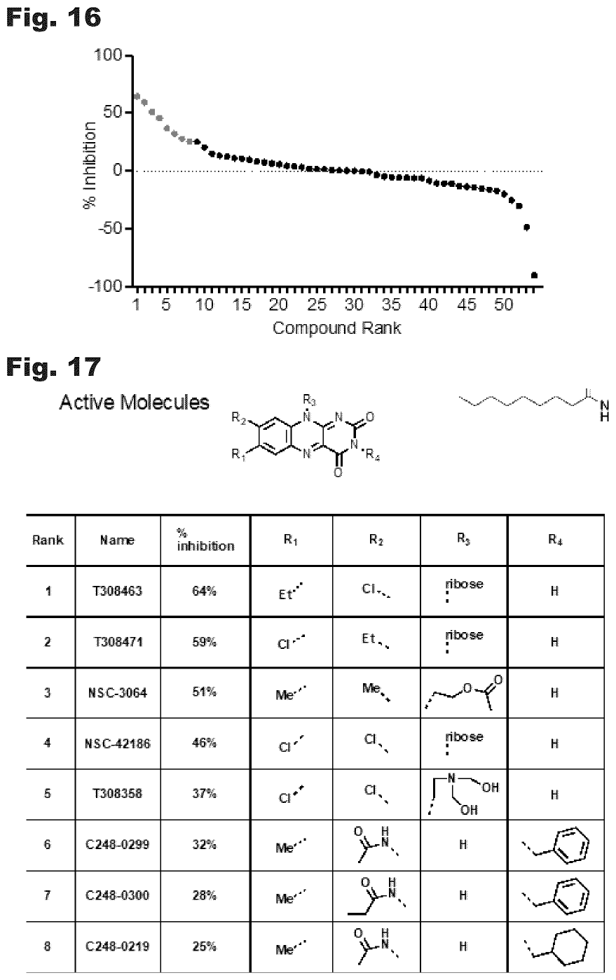

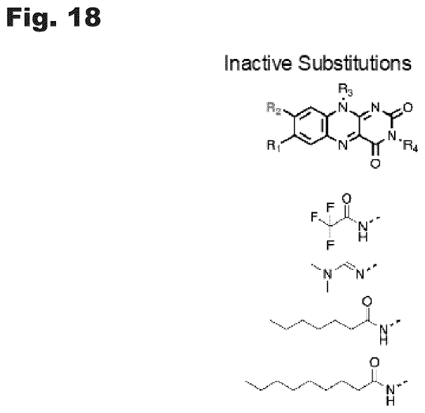

Based on the detection of multiple riboflavin analogs with HAT1 inhibitory properties we next screened a focused library of 54 compounds that all contained a core tri-cyclic ring structure similar to isoalloxazine (FIG. 16). This library contained molecules with R-group substitutions at four different sites around the isoalloxazine core (FIG. 17). R-groups from the top 8 most potent compounds are summarized in table format (FIG. 17). The top two compounds are highly similar with chloro- and ethyl-substitutions at either R₁ or R₂ positions and ribityl-groups at position R₃. This screen also identified several substitutions that dramatically impaired inhibitory activity, for example, bulky or aliphatic substitutions at R₂ (FIG. 18). In contrast, aromatic substitutions at R₄ were partially tolerated (FIG. 17). Therefore, this focused library of isoalloxazine derivatives allowed for determination of structural determinants associated with HAT1 inhibitory activity.

### Medicinal Chemistry Optimization of Riboflavin Analog 7-Chloro, 8-Ethyl-Isoalloxazine

Thus far the studies have identified a series of compounds based on the isoalloxazine tricyclic ring system that contains HAT1 inhibitory activity when modified at specific positions. The most potent inhibitors recovered included chloro- or ethyl-substitutions at the 7,8 positions and a sidechain at amino 10. Therefore, a medicinal chemistry approach was undertaken to synthesize a library of compounds that incorporated these features. Using a 7-chloro-, 8-ethyl-isoalloxazine core, analogs were synthesized with R-groups at the amino-10 side-chain position. Chemical synthesis of analogs was performed by amination of a nitrosylated di-chloro benzyl ring followed by condensation to yield the isoalloxazine core.

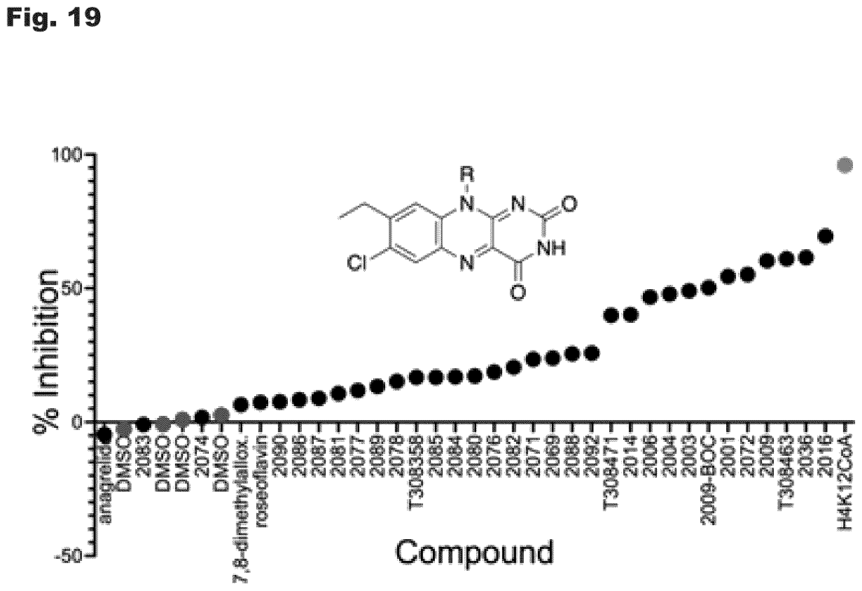

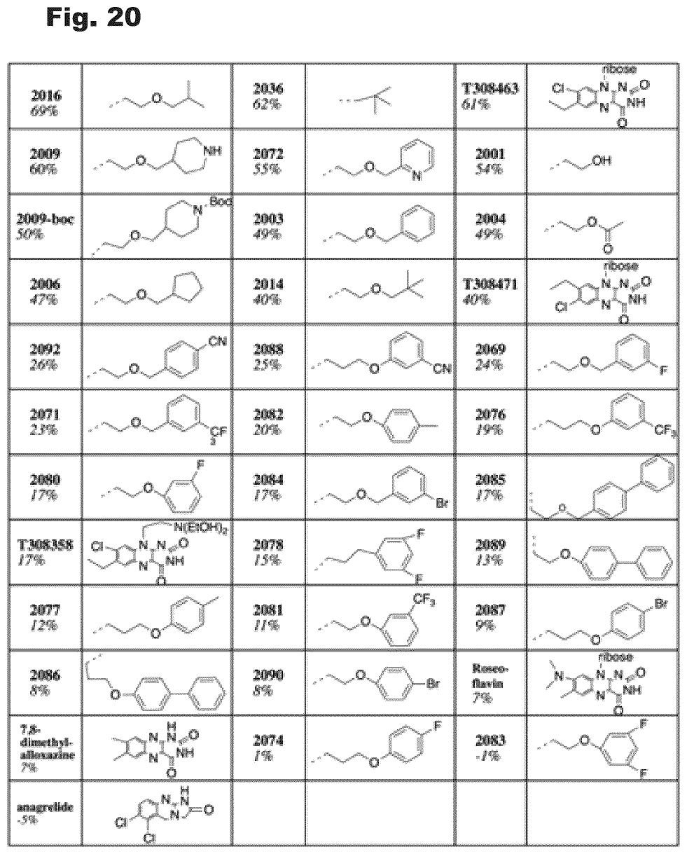

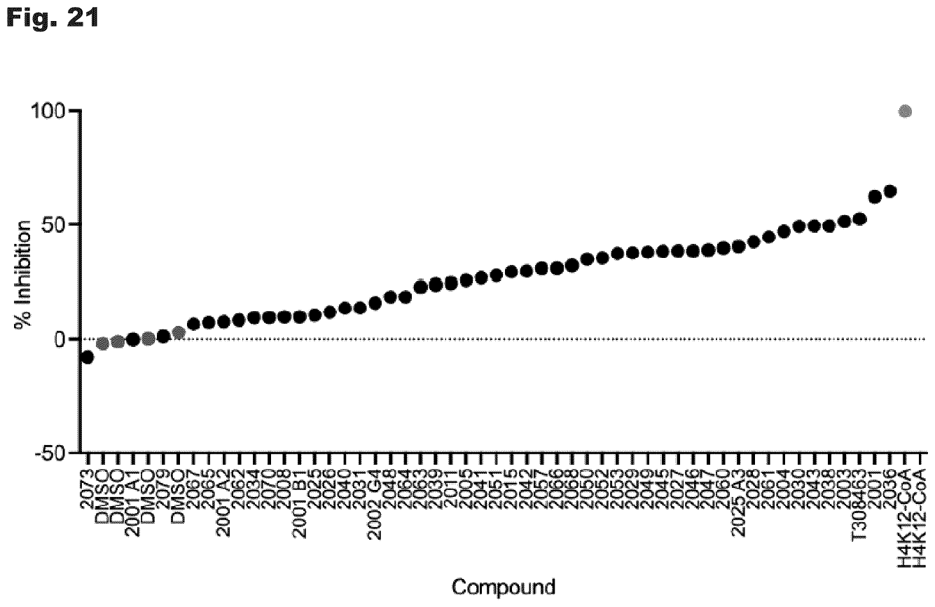

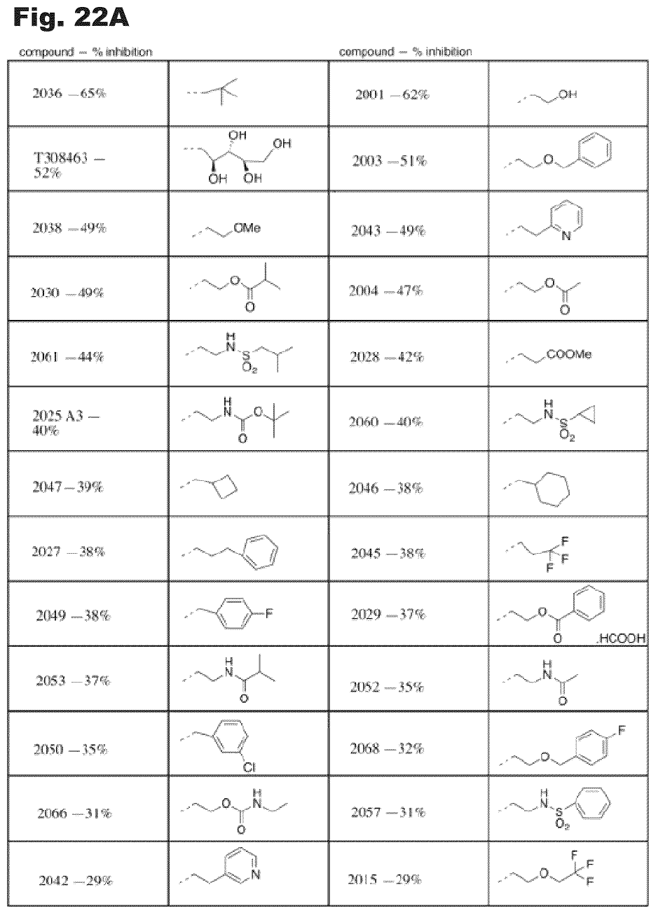

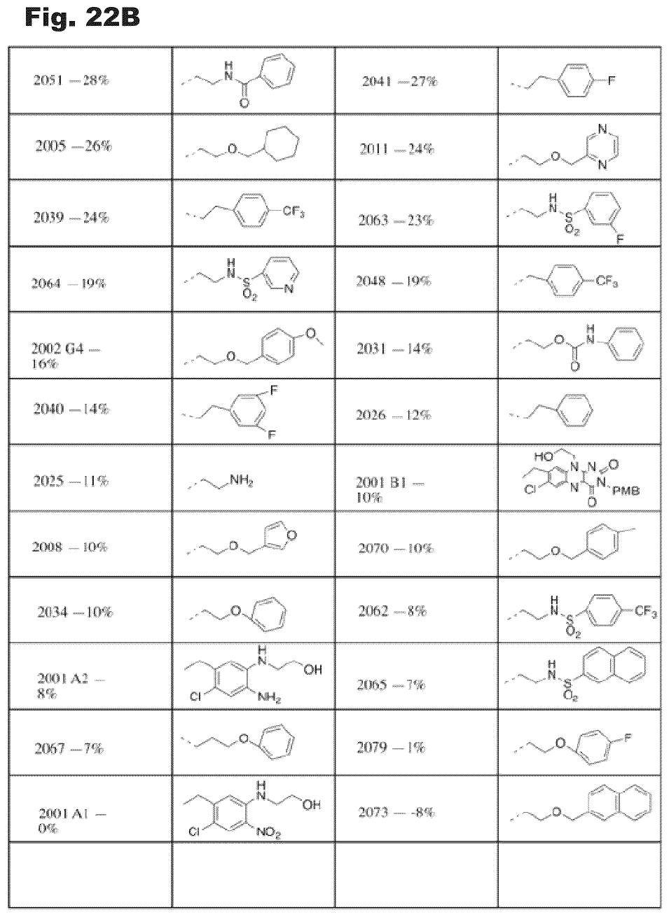

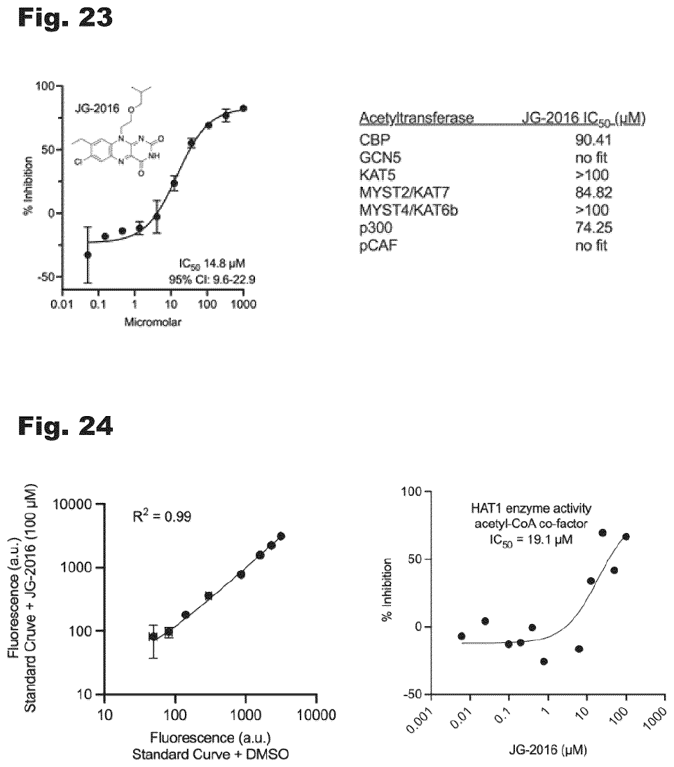

Altogether, 73 analogs were synthesized and tested for HAT1 inhibitory activity together with 11 additional compounds with structural similarity (84 compounds total). Of these 84 compounds, 11 (13%) caused at least 50% inhibition of HAT1 activity at 100 μM (FIGS. 19-22B). Single-dose inhibition studies identified analog JG-2016 as the most potent HAT1 inhibitor, which caused 69% inhibition at 100 μM (FIG. 19). This analog has a 1-ethoxy-2-methyl-propane sidechain at the amino-10 position of the isoalloxazine core (FIG. 20). Dose-response studies showed it to inhibit HAT1 enzymatic activity with IC₅₀ 14.8 μM (95% CI: 9.6-22.9 μM; FIG. 23, left panel), which is approximately a 2-4 fold improvement over the original hit compound NSC-42186.

In addition, this medicinal chemistry approach identified structure-activity relationships. For example, bulky sidechains interfered with enzyme inhibition, most prominently the 1-ethoxymethyl-benzene sidechains substituted at the meta and para positions in compounds 2092, 2088, 2069 and others (FIG. 20), suggesting a steric effect. In summary, these studies identify active and inactive amino-10 position sidechains with HAT1 enzyme inhibitor properties on an isoalloxazine scaffold.

### Mechanism of HAT1 Inhibition by JG-2016

These structure-activity studies nominated the analog JG-2016 for further study as it was the most potent HAT1 inhibitor identified in enzymatic studies. Synthesis of this molecule was scaled-up to the 5-gram scale, and its structure was confirmed by crystallization. To validate that this compound represented a true enzymatic inhibitor and not an assay-interfering compound due to the intrinsic fluorescence of the isoalloxazine core, further characterization of assay conditions in the presence or absence of the compound were performed. Standard curves were prepared in the presence of JG-2016 or equivalent amounts of vehicle (DMSO) as a control (FIG. 24, left panel). The fluorescence activity in the standard curve is generated by titrating a defined amount of biotinylated H4 n-terminal peptide with lysine 5 and 12 substituted for propargylglycines to mimic the HAT1-dependent acylation that occurs in the presence of 4-pentynoyl-CoA. This allowed assessment of the robustness of assay steps in the presence of JG-2016 including binding to neutravidin plates, copper-catalyzed azide-alkyne cycloaddition of biotin-azide, streptavidin-HRP binding and finally amplex red oxidation and fluorescence detection. The standard curves generated in the presence of JG-2016 were indistinguishable from control conditions (FIG. 24, left panel, R²=0.99). This indicates that JG-2016 does not interfere with these assay steps, and instead, likely disrupts HAT1-dependent peptide acylation.

The ability of JG-2016 to impair peptide acetylation more broadly was tested by assaying its inhibitory activity against seven other human histone acetyltransferases. JG-2016 had modest inhibitory activity towards CBP, MYST2/KAT7, p300 (IC₅₀ values 90.41, 84.82, 74.25 μM, respectively; FIG. 23, right panel) that were at least 5-fold higher than that observed towards the HAT1 complex. Additionally, JG-2016 had minimal or undetectable inhibitory activity towards GCN5, PCAF, KAT5 or MYST4/KAT6b. These results suggest that JG-2016 has specific activity towards the HAT1 complex, which is greater than that towards other tested human acetyltransferases. In addition, JG-2016 enzyme inhibition assays carried out in the presence of acetyl-CoA, instead of 4-pentynoyl-CoA, yielded similar dose-response relationships for HAT1 inhibition (FIG. 24, right panel; IC₅₀=19.1 μM).

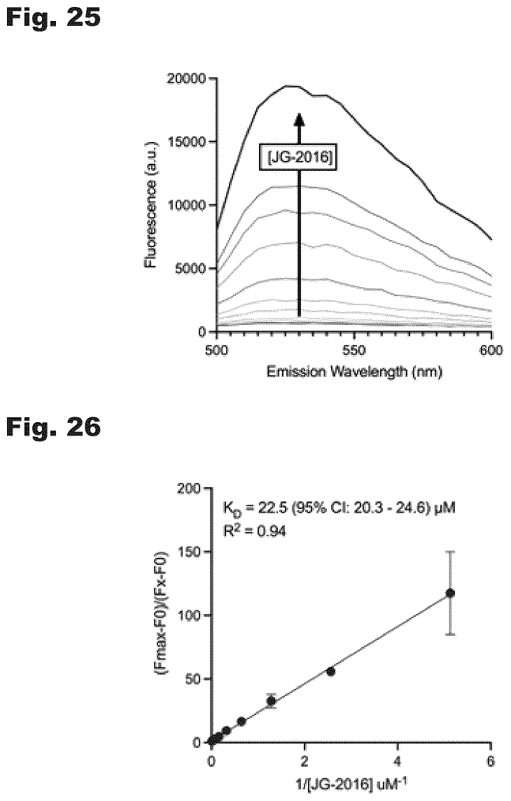

Next, assays for target engagement were performed to demonstrate that JG-2016 could directly modulate the HAT1:Rbap46 complex. The intrinsic fluorescence of JG-2016 was exploited to perform fluorescence titration experiments that the direct binding of the compound to the protein complex to be monitored (FIGS. 25-27). Riboflavin and analogs that retain the isoalloxazine core, including JG-2016 demonstrate broad excitation between 300-500 nm and emission between 500-600 nm. When excited at 472 nm, fluorescence emission scans (500-600 nm) demonstrated JG-2016 concentration-dependent fluorescence signal (FIG. 25). This led to more careful testing of the fluorescence characteristics of JG-2016 when mixed with HAT1.

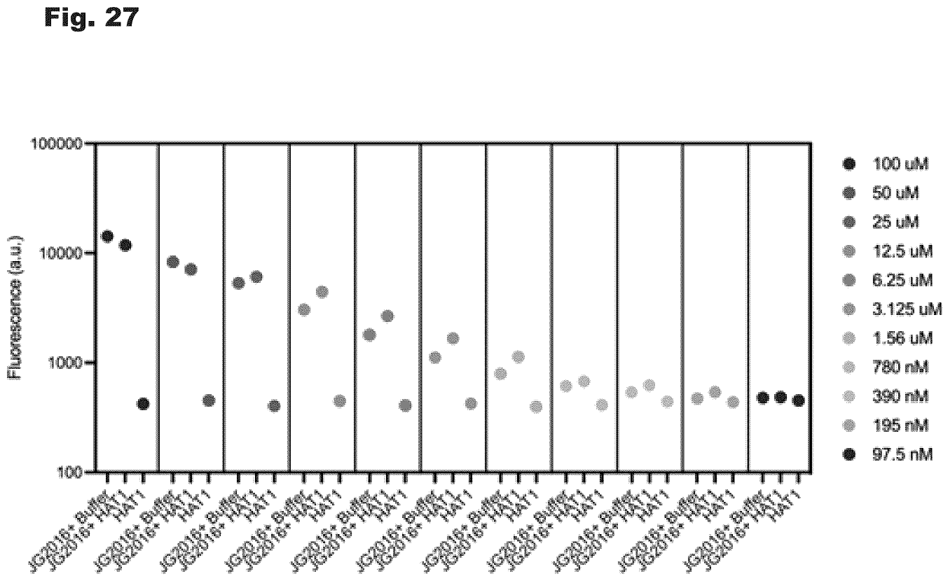

To determine if JG-2016 binding to HAT1 complex modulated fluorescence emission, the dose of JG-2016 was serially titrated from 100 to 0.0975 μM in constant concentration of the HAT1 complex (1.5 μM). There was increased fluorescence emission from JG-2016 in the presence of the HAT1 complex, whereas HAT1 in isolation had only background fluorescence (FIG. 27). A modified Benesi-Hildebrand equation was applied to derive the dissociation constant (K_{D}) for binding of JG-2016 to the HAT1 complex. The obtained K_{D} was 22.5 μM (95% CI: 20.3-24.6 μM; FIG. 26) which was in reasonable agreement with the observed IC₅₀ for enzymatic inhibition in the acetylation assays (FIG. 23, left panel; ˜15 μM). Thus, JG-2016 could directly bind to the HAT1 complex.

### Biological Activity of JG-2016

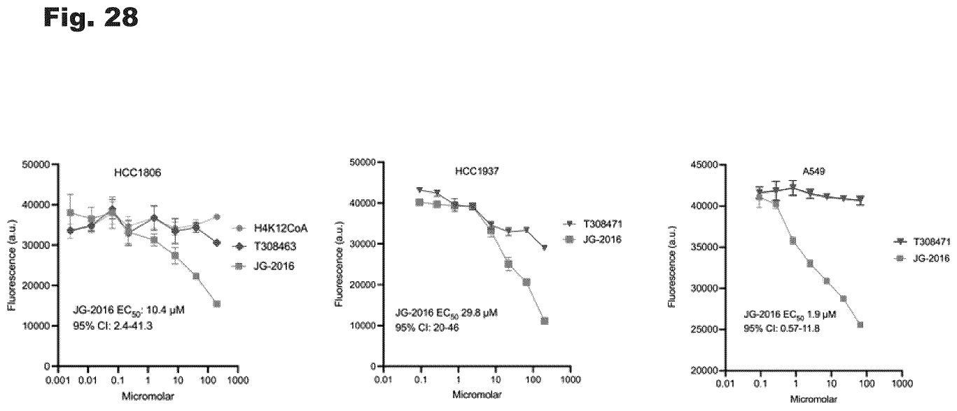

Given that JG-2016 appeared to have relative specific activity towards HAT1 compared to other human acetyltransferase enzymes the effects of JG-2016 in cell lines was assessed. The triple-negative breast cancer cell line HCC1806 was treated with JG-2016, H4K12-CoA and the riboflavin analog T308463 at varying doses and cell growth was assessed by addition of resazurin, which is reduced to resorufin in proliferating cells and emits fluorescence at 590 nm (FIG. 28, left panel). Although H4K12-CoA is a potent enzymatic inhibitor of HAT1 in cell-free assays, it does not cross cell membranes and is susceptible to metabolic degradation and thus had no inhibitory activity against this cell line. In contrast, JG-2016 robustly inhibited cell growth (EC₅₀ 10.4 μM; FIG. 28, left panel). Additionally, JG-2016 was a more potent cell growth inhibitor compared to the parent compound T308471 in HCC1937 (triple-negative breast cancer; EC₅₀=29.8 μM; FIG. 28, middle panel) and A549 (lung cancer; EC₅₀=1.9 μM; FIG. 28, right panel) cell lines. The weak inhibitory activity of the T308 compounds with 10-ribityl sidechains mirrors the minimal toxicity of riboflavin which can be tolerated at mega-doses in animals due to urinary excretion. Thus, the substituted isoalloxazine scaffold derivative JG-2016 has distinct biological properties compared to classical flavonoids.

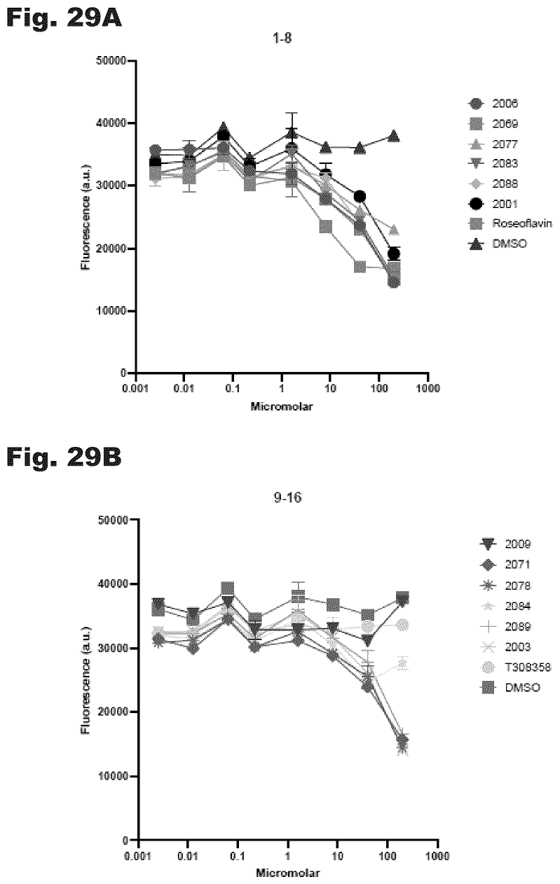

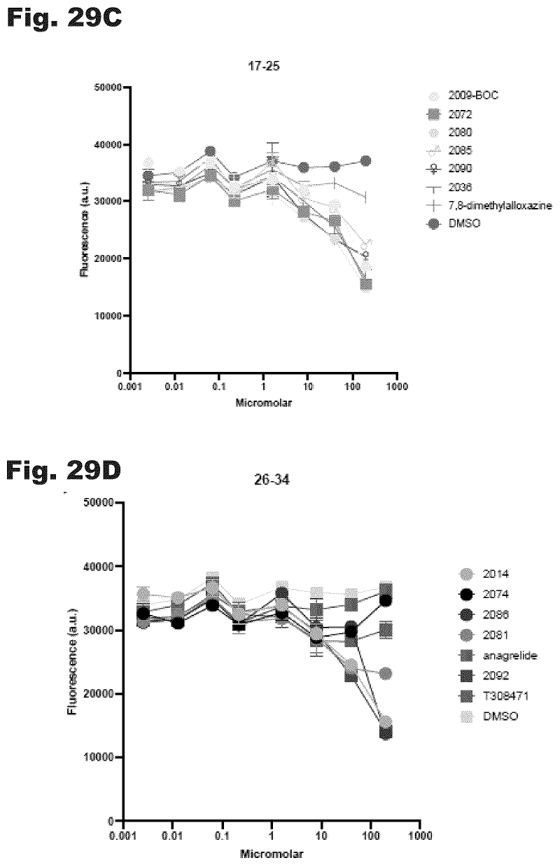

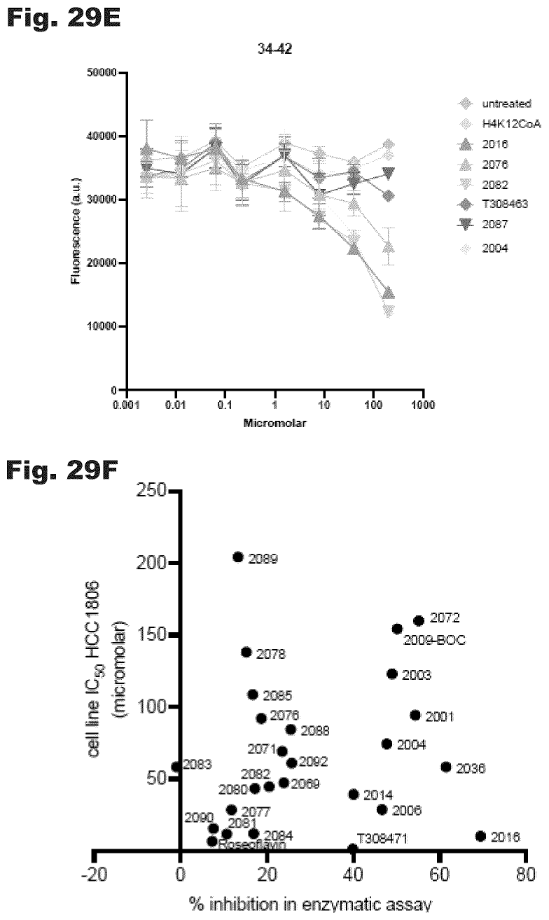

The EC₅₀ for growth inhibition was next assessed in the HCC1806 cell line for 42 flavonoids (selected for a range of HAT1 enzymatic IC₅₀s), as well as other isoalloxazine derivatives and control treatments (FIGS. 29A-29E). In these experiments, JG-2016 analog was among the most potent inhibitors of cell growth. In contrast, some analogs (e.g., roseoflavin) had strong cell growth inhibitory properties but were inactive in the HAT1 enzymatic assay. Among all tested compounds, the correlation of cellular EC₅₀ for cell growth versus enzymatic IC₅₀ for inhibition demonstrated that JG-2016 had the most optimized combination of these parameters (FIG. 29F).

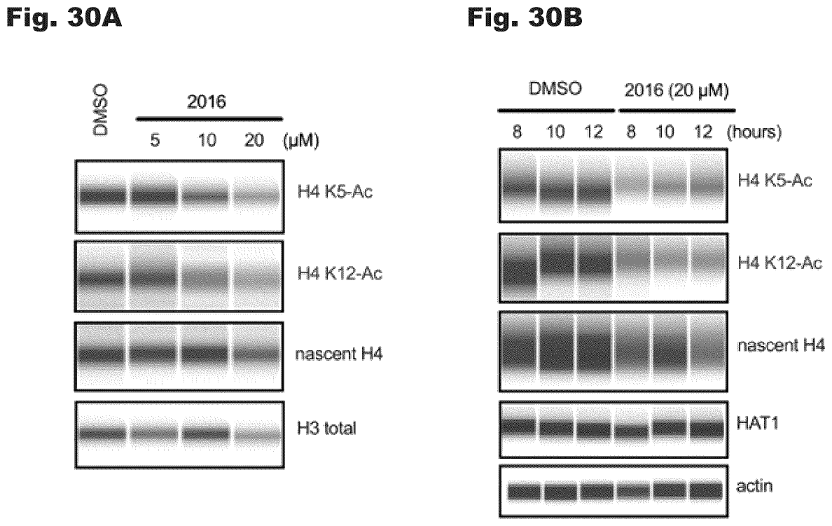

Given the properties of JG-2016 as an enzymatic inhibitor of HAT1 and a suppressor of cell growth it was next tested if this compound could inhibit HAT1 acetylation in vivo. It was previously demonstrated that acute suppression of HAT1 protein levels led to decreased H4 lysine 5 and 12 acetylation of nascent histone tetramers, as well as decreased total H4 protein levels in the hTert-HME1 cell line (J. J. Gruber, et al., Mol Cell 75:711-724 e5, 2019, cited supra). When these cells were treated with JG-2016 we observed dose-dependent inhibition of H4 lysine 5 and 12 acetylation by site-specific antibody-based capillary immunoassay (FIG. 30A). Similarly, time-course analysis also showed on-target inhibition as early as 8 hours post-treatment that persisted through 12 hours (FIG. 30B). At some timepoints, decreased H4 protein levels were also observed, consistent with prior studies of the effects of HAT1 depletion (J. J. Gruber, et al., Mol Cell 75:711-724 e5, 2019, cited supra). These experiments indicated that treatment with JG-2016 can impair HAT1 dependent acetylation in cells.

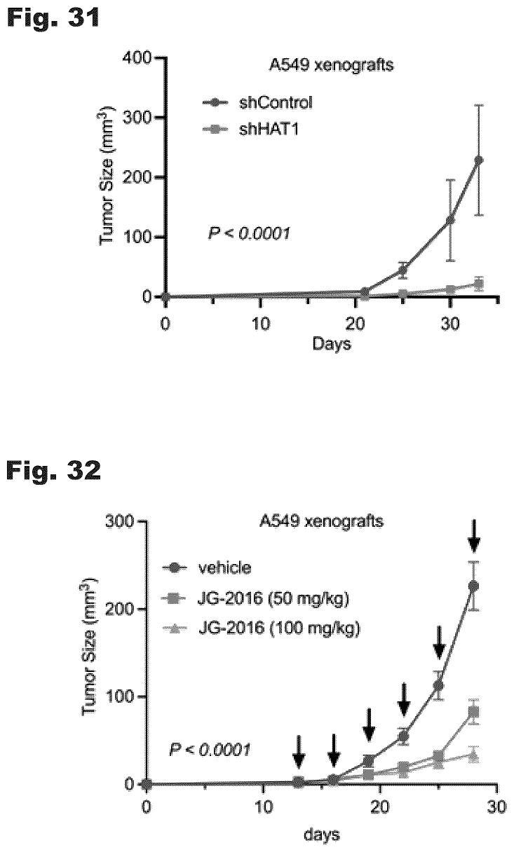

As JG-2016 treatment could decrease nascent H4 acetylation in cells, it was tested whether JG-2016 treatment could impair tumor growth in pre-clinical mouse models. The A549 model was chosen because of the low IC₅₀ required to impair cell proliferation by JG-2016 treatment (1.9 μM; FIG. 28, right panel). First, the A549 cell line was treated with HAT1-targeted shRNAs to demonstrate that HAT1 was required for tumor growth. A549 cells treated with shRNAs targeting the HAT1 mRNA evinced suppression of tumor growth in mice compared to cells treated with control shRNAs (p<0.0001; FIG. 31). Next, A549 cells were implanted into flanks of mice and allowed to establish for 13 days, then treated with intraperitoneal injections of JG-2016 (FIG. 32). Dose-finding studies showed that mice could tolerate up to 250 mg/kg or lower as a single intraperitoneal injection with minimal toxicity, but signs of distress were observed at doses of 500 mg/kg. Thus, two dose levels of JG-2016 were used: 50 mg/kg and 100 mg/kg, both delivered once every three days. These doses are similar to murine dosing of other targeted inhibitors used for cancer. Treatment with JG-2016 at both doses significantly impaired tumor growth compared to vehicle-treated control mice (p<0.0001; FIG. 32). A dose-response relationship was observed with the higher dose causing more profound suppression of tumor growth compared to the lower dose. This indicates that JG-2016 has anti-tumor activity in a pre-clinical model.

### Significance of Data Results

Multiple reports have suggested that HAT1 may be a cancer therapeutic target based on protein knockdowns or knockouts in various pre-clinical models. These works motivated the search for small molecules capable of interfering with HAT1 enzymatic activity. Here, a platform was developed to identify and characterize HAT1 acetyltransferase activity based on a high-throughput, peptide-based, click-chemistry-enabled enzymatic assay. The advantages of this enzymatic assay include the utilization of the human HAT1/Rbap46 enzyme complex purified from human cells as opposed to a bacterial source. Also, this assay provides a direct readout of enzymatic activity on the peptide substrate without relying on coupled reactions that are prone to nonspecific inhibition. In addition, its high-throughput characteristics in 96-well plates were validated, which should enable larger chemical screens to be performed. The click chemistry approach based on 4-pentynoyl-CoA as an acetyl-CoA analog should be adaptable to other acetyltransferases as well. Mutations shown to improve 4-pentynoyl-CoA utilization have recently been reported, which could be incorporated into future studies. Finally, as reaction products are bound prior to functionalization and quantification, it allows for washing to remove potential assay-interfering compounds that commonly cause nonspecific signatures in other assays.

JG-2016 was prioritized based on a workflow that spanned virtual structure-based docking algorithms followed by enzymatic assays, focused library screening, medicinal chemistry and biologic assays. This compound retains the isoalloxazine core common to flavonoids with modifications to the 7,8, and 10 positions that led to significant improvements in enzyme inhibition and cellular growth inhibition. Prior work has demonstrated that flavonoids are selectively transported into cancer cell lines, indicating that active transport of JG-2016 may be a useful feature for cancer-specific targeting. A family of riboflavin transporters have recently been identified and shown to specifically recognize and transport the isoalloxazine core, rather than the ribityl sidechain. Thus, JG-2016 retains chemical features that allow for its transport into cells. Cancer-specific expression of riboflavin transporters may be a biomarker of sensitivity to this agent. This also raises the possibility of using isoalloxazine as a mechanism to achieve cancer-selective targeting of therapeutic agents.

HAT1 sits at the intersection of cytoplasmic mitochondrial processes that generate acetyl-CoA and nuclear reactions that consume it to drive transcription of growth programs. HAT1 likely functions as a cytoplasm-to-nucleus acetyl-shuttle by acetylating nascent histones in the cytoplasm that then become rapidly de-acetylated upon insertion into chromatin leading to a nuclear acetyl pool. This work describes isolation of the first small molecule compounds capable of modulating HAT1 enzymatic activity. These compounds may serve as chemical tools to further our understanding of HAT1 biology, its role in chromatin synthesis and the connection between cellular metabolism, epigenetics, and nuclear acetyl flux. JG-2016 or other related analogs may also serve as a chemical scaffold for more potent HAT1 inhibitors through structure-based design or chemical similarity screens. Finally, this work demonstrates that HAT1 yields therapeutic vulnerability in cancers with an acceptable toxicity profile. Given the importance of HAT1 in the response to EGF stimulation and its role in histone maturation and cell cycle progression, this indicates that further efforts to target HAT1 may be fruitful for cancer therapy.

### EXPERIMENTAL METHODS

### Buffers

Buffers utilized in experimentation include:
RSB-500: 20 mM Tris/pH7.5, 500 mM NaCl, 25 mM MgCl2-keep at 4 deg C.
   500 mL=10 mL 1M Tris, 50 mL 5M NaCl, 1.25 mL 1M MgCl2-H2O to 500 mL
RSB-100: 20 mM Tris/pH7.5, 100 mM, NaCl 25 mM, 25 mM MgCl2-keep at 4 deg C.
   500 mL=10 mL 1M Tris, 10 mL 5M NaCl, 1.25 mL 1M MgCl2-H2O to 500 mL
Elution Buffer: (25 mM HEPES pH 8.0, 100 mM NaCl, 0.01% Triton X)
20×HAT1 Buffer (1 M Tris pH 8.5, 0.1% NP40 [IGEPAL])
PBST: PBS with 0.1% Tween-20

### Expression and Purification of HAT1 Complex

Full-length human HAT1 and Rbap46 were independently cloned into the pHEK293 vector (Takara Bio) with Gibson assembly. HAT1 was appended with a C-terminal FLAG tag, Rbap46 was unmodified. The pHEK293 vector was digested with Xbal then amplified with Phusion polymerase (Thermofisher) and primers, then gel purified. HAT and Rbap46 were ordered as gBlocks (IDT) with ˜20 bp ends overlapping sequence with the pHEK293 vector. Gibson assembly was performed with NEBuilder HiFi DNA Assembly (NEB) and propagated in DH5a cells. 293F cells (Thermo-Fisher) were grown in suspension culture in a humified incubator with 8% CO2 at 37deg C. with constant shaking at 120 RPM. 293F were seeded to a density of 5E5 cells/ml in 300 ml culture volume in a 1 L baffle-free plastic Erlenmeyer flask with vented caps. The next day the transfection mix was prepared with 300 μg of pHEK293-HAT1-FLAG and 300 μg of pHEK293-Rbap46 in 30 mL of PBS with 1.2 mL of PEI (0.5 mg/ml), incubated for 15 minutes, then added to 300 mL culture of 293F. After 48 hours, cells were treated with 12.5 μM forskolin for 30 minutes at 37deg C. then collected by centrifugation and snap frozen. Cell pellets were lysed in 40 mL of RSB-500 buffer with 0.1% triton-X-100 with Complete protease inhibitors (Roche). Lysate was sonicated at 10% amplitude for 10 seconds×3, then centrifuged at 10K RPM for 10 minutes. Supernatant was collected and immunoprecipitated with FLAG M2 agarose (400 μL per 10 mL of extract) for 2 hours, then washed extensively in lysis buffer, then once in RSB-100 buffer+0.1% triton-X-100. HAT enzyme was eluted with FLAG peptide diluted to 0.5 mg/mL in EB (1.5 mL) for at least one hour at 4deg C. Supernatant was collected and combined, washed twice with 15 mL EB and concentrated by centrifugation in Amicon 10K cutoff filters and resuspended in 6 mL EB per original 300 mL culture volume. Agilent protein 230 chip was run to quantitate protein concentration, then snap frozenin 200 μL aliquots and stored at −80deg C. HAT1 acetylation assays were performed to validate enzyme activity and then enzyme diluted in EB to yield approximately 1500 fluorescence units (25% full activity on standard curve) and re-frozen. Typically, this was a 1:40 dilution.

### Virtual Drug Screening Workflow, Docking, Similarity Searches, Visualization

Schrodinger software (2018-3) was utilized to prepare the HAT1 crystal structure 2P0W for serving as a target model for virtual screening. The cognate ligand (acetyl-CoA) was used to generate the grid for the virtual screen. SDF file (NCI_Open_2012-05-01.sdf.gz) of the NCI/DTP open compounds was downloaded from https://cactus.nci.nih.qov/download/nci/. Using the Schrodinger Virtual Screening Workflow pipeline, the ligands were prepared by desalting, removing duplicates and generating conformers (max 32) at pH 7.0-7.2 using EPIK algorithm. The ligands were docked into the receptor grid in 3 successive steps of increasing precision levels (HTVS, SP, XP) with Schrodinger Glide program. The resulting docked poses were used to estimate the binding affinity of the ligand using MMGBSA method as implemented in Schrodinger. The ligands that had favorable Glide XPScore (<10.5) and MMGBSA DGBIND (<−60) were prioritized for testing in the experimental assay. The initial hit compound (NSC-42186) was used as a template to find similar compounds from the NCI database using Schrodinger shape screen. The physiochemical properties of the resulting compounds were calculated using qikprop (Schrodinger) then filtered for predicted water solubility (QPlogS <−1.0 & >−5.7) and reactive functional groups (#rtvFG=0). For visualization docking was performed with mcule centered on the methionine 241 in the cofactor binding site and visualized in pymol. Electrostatic surface potential was calculated in pymol with the APBS plugin.

### HAT1 Acetylation Assay

Acetylation reactions were assembled from the following components: histone H4 peptide (1-23-GGK-biotin; Anaspec #AS65097) resuspended in DMSO to 0.1 mg/mL [34.8 μM], HAT1 enzyme pre-diluted in EB, 20× buffer, 2 mM DTT, 4-pentynoyl-CoA dissolved in water to 1 mg/mL [1 mM]. A 20 μL reaction comprised 10 μL of enzyme, 1 μL of H4 peptide, 1 μL of 20× buffer, 1 μL of DTT pre-mixed and aliquoted to wells of a 96-well PCR plate on ice. Then 1 μL of DMSO or test compound dissolved in DMSO was added per well and mixed by gentle pipetting and allowed to incubate for 10 minutes on ice. Then 2 μL of 4-pentynoyl-CoA and 4 μL of water were pre-mixed and added together to wells, which were then gently mixed by pipetting, centrifuged briefly at 2500 RPM to collect contents, and incubated at 37deg C. for 1 hour. Contents were then either directly processed for reaction products or quenched with 20 μL of 8M urea and stored at −20deg C. until processing. Reaction contents were added to BSA pre-blocked black Neutravidin coated 96-well plates (Fisher #15217) containing 80 μL of PBST per well and bound with gentle orbital shaking for 1 hour at room temperature. Wells were washed 200 μL PBST 3×15 strokes (180 μL stroke volume) with a Hydra96 (Art Robbins Instruments). Click reactions were assembled as follows:
During Peptide binding prepare click reagents fresh
   100 mM THPTA ligand in aqueous buffer or water (4.35 mg/100 uL)
   20 mM CuSO4 in water (4.99 mg/mL)
   300 mM sodium ascorbate in water (29.7 mg/500 uL)
   2.5 mM Biotin Azide (stock is 2.5 mM)
One click reaction contains (scale appropriately to the number of reactions needed):
   140 uL PBS
   10 uL of THPTA
   10 uL of CuSO4
   10 uL of Na Ascorbate
   20 uL of Biotin Azide

Click reagents were dispensed to each well and incubated at 37deg C. for 1 hour, then plate washed 3× as before. Dilute Strept-HRP (CST #3999S®0.224 mg/m) 1:10 in Streptavidin (0.224 mg/ml; EMD Millipore #189730), then further dilute this mix 1:1000 in PBST. Add 100 uL per well then incubate at room temp for 1 hour with gentle orbital shaking. Wash 3× with PBST, then add amplex red detection reagents:
4.45 mL NaHPO4 buffer (1×) (50 mM final Concentration)
50 uL Amplex Red (20 mM diluted in DMSO)
500 uL of dilution H2O2
   Dilution of H2O2 (30% stock)
      3% H2O2-Dilute 3 uL of stock H2O2 in 27 uL of 1×NaHPO4 buffer
      Add 22.7 uL of that 3% H2O2 to 977 uL of 1×NaHPO4 (this is the H2O2 to add to mix)

Add 100 uL of amplex red reaction mix per well, incubate at room temp for 30 minutes protected from light then read fluorescence excitation/emission 571/585 nm. Percent inhibition was calculated according the following formula:

$$\frac{D - X}{D - BG}$$

where D is the fluorescence value of control reactions treated with DMSO only, X is value of reaction treated with test compounds, and BG is value of background wells (no enzyme added). Least squares regression was used to fit dose-response and competitive versus non-competitive inhibition curves.

For HAT1 acetylation assays with acetyl-CoA, identical reactions were carried out with acetyl-CoA in place of 4-pentynoyl-CoA. Then reaction products were spotted onto nitrocellulose membranes and dot blotted with anti-H4-lysine-12 antibodies. Immunoblot signal was quantified by densitometry.

A positive control H4 N-terminal peptide was synthesized (Genscript). Peptide was resuspended at 0.1 mg/mL in DMSO, then mixed with H4 N-terminal peptide (Anaspec #AS65097) to create a standard curve. These mixtures were bound to neutravidin plates, functionalized by click chemistry, bound with streptavidin-HRP and reacted with amplex red as described above for the HAT1 acetylation assay. LoD was calculated as the assay baseline (negative controls) plus 3× the standard deviation of the baseline measurements. LoQ was calculated as the assay baseline plus 10× the standard deviation of the baseline measurements.

### Other Acetyltransferase Assays

Acetylation assays on 7 targets (CBP, GCN5, KAT5, MYST2/KAT7, MYST4/KAT6b, p300, pCAF) was performed by Creative Biomart. JG-2016 was tested in a 10-dose IC50 mode in singlet with 3-fold serial dilution, starting at 100 μM. Reaction buffer was 50 mM Tris-HCL, 0.1 mM EDTA, 250 mM NaCl, 1 mM DTT, 1% DMSO, pH 8.0, using 3 μM 3H-Acetyl-CoA, reaction time of 1 hour at 30 degrees Celsius, with substrate conversion rate between 5-20%. Enzymes were diluted in reaction buffer, followed by addition of compound with Acoustic Technology in nanoliter range, incubated at room temperature for 20 minutes, then 3H-Acetyl-CoA was added. Reaction was incubated at 30 minutes at 30 degrees Celsius, then transferred to filter paper for detection.

### Fluorescence Titration

Fluorescence measurements were performed using a Clario Star microplate reader with black 384 well plates (Greiner, small volume). JG-2016 was diluted in Elution Buffer (100 μM-97.5 nM, serial 2-fold dilutions) and separately in HAT1 enzyme complex (1.5 μM) pre-diluted in Elution Buffer (JG-2016: 100 μM-97.5 nM, serial 2-fold-dilutions). As a control HAT1 enzyme complex (1.5 μM in Elution Buffer) without JG-2016 was also prepared. JG-2016 stock concentration was 20 mM in DMSO and initially diluted 1:200 into either HAT1-containing buffer or buffer alone (thus top DMSO concentration was 0.5%). Ten microliters of each sample were added to the 384 well plate, centrifuged for 1 minute at 1000×g, then fluorescence was read in excitation/emission mode. Plate reader settings: Focal height 14 mm; Excitation settings: excitation bound 320-470 nm, resolution 5 nm, dichroic auto-set, and emission wavelength 548 nm; Emission settings: emission bound 500-600 nm, resolution 5 nm, dichroic auto-set, and excitation wavelength 472 nm. Scans were performed using top optics and precision mode. Monochromator settings: [320-14->470-14/548-100] gain=1831; [472-16/500-10->600-10] gain=1929. The modified Benesi-Hildebrand equation was applied:

$$\frac{{F}_{max} - {F}_{0}}{{F}_{x} - {F}_{0}} = 1 {K}_{D} ( \frac{1}{{[ M ]}_{n}} )$$

where [M]=concentration of JG-2016 in micromolar; Fmax=fluorescence count of JG-2016 at 100 μM in HAT1-containing buffer at 525 nm; F0=average of fluorescence counts for HAT1-containing buffer alone at 525 nm; Fx=fluorescence count of JG-2016+HAT1-containing buffer at [M] concentration.

### Cell Culture, Drug Treatments, Immunoassays

hTert-HME1, HCC1806, HCC1937, A549 were maintained in a humified incubator at 37deg C. with 5% CO₂. HCC1806 and HCC1937 were grown in RPMI with 10% FBS and 1% penicillin-streptomycin. A549 was grown in F-12 media with 10% FBS and 1% penicillin-streptomycin. hTert-HME1 was grown in Mammary Epithelial Growth Media (PromoCell). Drug treatments were performed in 96-well plates. Cells were seeded at 5000 cells per well, then allowed to attach overnight. Drug dilutions were made in 96-well plates then transferred to plate containing cells and incubated for 48-72 hours. Cell density was then determined by CellTiter-Blue (Promega). For capillary immunoassays hTert-HME1 5E5 cells were seeded in 10 cm plates and cultured for 48 hours, then cells were washed 2× in PBS and EGF-free MEGM was added overnight. The next day, cells were treated in EGF-free media with 1% dialyzed BSA+drug for 30 minutes, then EGF was added to the plate and cells were cultured for 8, 10 and 12 hours before harvesting. Microcapillary immunoassays were performed on a SimpleWestern Wes instrument using H4K12-Ac antibody (Abcam ab46983) at 1:25 dilution, H4K5-Ac antibody (Millipore 07-327) at 1:50 dilution, HAT1 (Abcam ab194296) at 1:50 dilution, nascent H4 (Abcam ab7311) at 1:10 dilution, nascent H3 (Abcam ab18521) at 1:50 dilution, actin (Thermo-Fisher MA5-15452) at 1:100 dilution.

### Mouse Models

All mouse experiments were conducted with prior approval of the Administrative Panel on Laboratory Animal Care. A549 cells were infected with 3 lentiviral shRNAs targeting the human HAT1 mRNA (Origene #TL312517). Six-to-eight-week-old NSG female mice (Jackson Labs #005557) were shaved, then 500,000 A549 tumor cells were injected into bilateral flanks and tumor growth was assessed by tri-dimensional tumor measurements to yield tumor volumes. For drug treatments, analog 2016 was resuspended in a 1:1 mixture of PEG-400 and PBS+1% dialyzed BSA-0.2 μm-filtered (MilliporeSigma #12-660-910GM). Intraperitoneal injections (200 μL) were performed with a 21-G needle. Tumor measurements were performed with digital calipers.

### Chemical and Synthesis

Riboflavin analogs were purchased from Chemdiv. For chemical synthesis of the 7-chloro-, 8-ethyl-isoalloxazine compound library, three general schemes were pursued. In the first, R-group amine side chain was either synthesized or purchased and used for nucleophilic addition of the common di-chloro-nitrosyl-ethyl-benzene (JG-2001-X), followed by reduction of the nitrosyl group to amine and then condensation with alloxan to generate the isoalloxazine core. In a second synthetic route (eg. for JG-2031 and JG-2029), the precursors JG-2001 or the PMB-protected analog JG-2001-B1 underwent either (1) esterification of free alcohol in the presence of triethylamine or (2) EDC, HCl+HOBt coupling in the presence of DMF and DMAP. In the third route, the free amine sidechain of compound JG-2025 was coupled to carboxylic acid sidechains in the presence of HATU, DIPEA and DMF (eg. to make JG-2051). Alternatively, JG-2025 was coupled to sulfonyl chloride containing sidechains in the presence of base (TEA or K2CO3) and DMF (eg. to make JG-2060). Compounds were purified by chromatography and validated by proton NMR and mass spectrometry.

### NMR, Mass Spectrometry, Compound Anaysis

¹H-NMR and ¹³C-NMR spectra were measured on a Bruker 400 MHz spectrometer (i-probe 5 mm with Topspin 3.2. Software). All 13C-NMR were recorded at 100 MHz. All purified products were determined to be ≥95% pure unless otherwise noted.

LCMS or mass analysis was performed using either of the following LCMS machines:
(a) Waters Acquity Ultra performance LC equipped with PDA and attached with QDA detector with a Waters X-bridge C18, 50*2.1 mm, 2.5-micron column using a binary solvent system [A: 0.1% FA in Water, B=0.1% FA in H2O: ACN (10:90)].
(b) Water Acquity UPLC-H Class equipped with PDA and Acquity SQ detector with a Waters X-bridge C18, 50*2.1 mm, 2.5-micron column using a Quaternary solvent system [A: 0.1% FA in Water, B=0.1% FA in H2O: ACN (10:90)].
(c) Waters Acquity Ultraperfomance LC connected with PDA and equipped with SQ detector with a Waters X-bridge C18, 50*4.6 mm, 3.5-micron column using a Binary solvent system [A=5 mM Ammonium Bicarbonate in H2O and B=ACN].

Mass analysis was performed on a Waters Acquity Ultraperfomance LC equipped with SQ detector using a Binary solvent system [A=5 mM Ammonium Acetate and 0.1% Formic acid in H2O and B=Methanol].

### Crystallization and Structure Determination

Crystals suitable for x-ray diffraction were obtained evaporating a solution of JG-2016 in isopropanol at room temperature. Data were collected at beamline SSRL BL12-2 at the Stanford Synchrotron Radiation Lightsource synchrotron at SLAC National Accelerator Laboratory (2572 Sand Hill Road, Menlo Park, California, CA 94025, USA). Except two hydrogen atoms attached to nitrogen, all the H atoms were refined using a riding model while keeping their isotropic displacement parameters constrained to 1.2 (H attached to aromatic C atoms) and 1.5 (H atoms attached to non-aromatic C atoms) times larger than those of their carrier atoms. Software used for structure analysis: XDS (Kabsch, 2010), WinGX (Farrugia, 1999), and SHELX (Sheldrick, 2008). Software for data collection Blu-Ice (McPhillips et al., 2002).

### Crystallization Data

| formula | ′C18 H21 CI N4 O3′ |
|---|---|
| weight | 376.84 |
| system | monoclinic |
| space group | ′P 21/c′ |
| unit cell parameters | |
| a | 15.5530(10) |
| b | 9.2970(10) |
| c | 26.0360(10) |
| alpha | 90 |
| beta | 104.670(10) |
| gamma | 90 |
| Z | 8 |
| measurement | 100(2) |
| temperature | |
| R | 0.0593 |
| GOF | 1.055 |

### Preparation of 7-chloro, 8-etyl-isoalloxazine core library compounds

### Synthesis of intermediate JG-2002-X

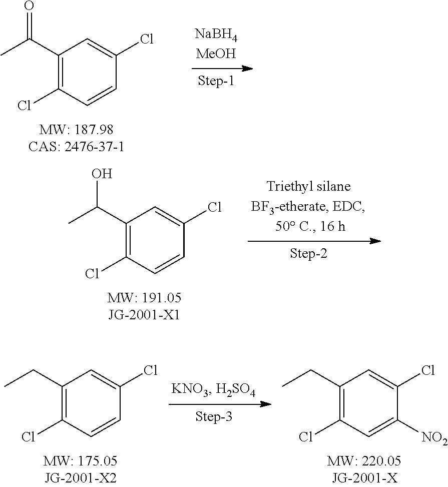

### Step-1: Synthesis of 1-(2,5-dichlorophenyl)ethan-1-ol

Batch no. STL7-A-588-JG-2001-X1-163-b

To a solution of 1-(2,5-dichlorophenyl)ethan-1-one (50.0 g, 264.4 mmol) in MeOH (500 mL), sodium borohydride (15.0 g, 396.7 mmol) was added at −10° C. and reaction mixture was allowed to stir at 0° C. for 1 h. After completion of reaction as indicated by TLC, the reaction mixture was concentrated carefully and crude was diluted with water and EtOAc. The reaction mixture was partitioned between water (1000 mL) and EtOAc (1000 mL×2). The organic layer was separated and washed with brine (2×1000 mL). The combined organic layer was evaporated under vacuo to obtain title compound as off-white solid (54.0 g, 77.63%). 1H NMR: (400 MHz, DMSO) δ 7.59-7.58 (t, J=4 Hz, 1H), δ 7.44-7.42 (dd, J=8 Hz, 1H), δ 7.35-7.32 (t, J=12 Hz, 1H), δ 5.55-5.54 (d, 1H), δ 4.99-4.95 (q, 1H), δ 1.31-1.30 (d, J=4 Hz, 3H).

### Step-2: Synthesis of 1,4-dichloro-2-ethylbenzene

Batch no. STL7-A-588-JG-2001-A2-167-C

To a solution of 1-(2,5-dichlorophenyl)ethan-1-ol (JG-2001-X1) (54.0 g, 284.2 mmol) in ethylene dichloride at 0° C., BF3-etherate (67.1 mL, 539.9 mmol) and triethylsilane (92.0 mL, 568.4 mmol) were added at 0° C. and stirred for 16 hr at 50° C. After completion of reaction as indicated by TLC, the reaction mixture was cooled at 0° C. and quenched with sat. NaHCO₃ (1500 ml). The reaction mixture was partitioned between EtOAc (1500 mL×2) and the organic layer was washed with brine solution (2×1500 mL). The combined organic layer was dried over Na2SO4 and evaporated to obtain title compound as pale-yellow oily liquid (45.0 g, quantitative). 1H NMR: (400 Mz, DMSO). δ 7.50-7.43 (m, 2H), δ 7.31-7.28 (dd, J=12 Hz, 1H), δ 2.72-2.66 (q, J=8 Hz, 2H), δ 1.24-1.14 (t, 3H).

### Step-3: Synthesis of 1,4-dichloro-2-ethyl-5-nitrobenzene

Batch no. STL7-A-588-JG-2001-X-178-c

To a solution of 1,4-dichloro-2-ethylbenzene (JG-2001-X2) (45.0 g, 258.6 mmol) in H2SO4 (450 mL) at 0° C., KNO3 (26.1 g, 258.6 mmole) was added at 0° C. and stirred for 30 min. After completion of reaction as indicated by TLC, the reaction mixture was quenched with ice water (1000 mL) slowly and stirred for 20 min. The product was extracted with EtOAc (500 ml×2) and washed with brine solution (2×500 mL). The combined organic layer was dried over Na2SO4 and evaporated under vacuum. The crude product was purified by column chromatography (0-15% EtOAc/hexane) to obtain title compound as pale-yellow oil (35.0 g, 56.89%). 1HNMR: (400 Mz, CDCl3) δ 1.20-1.16 (t, 3H), 2.792-2.735 (q, 2H), 7.799 (s, 1H), 8.23 (s, 1H).

### Synthesis of JG-2001: 7-chloro-8-ethyl-10-(2-hydroxyethyl)benzo[g]pteridine-2,4(3H, 10H)-dione

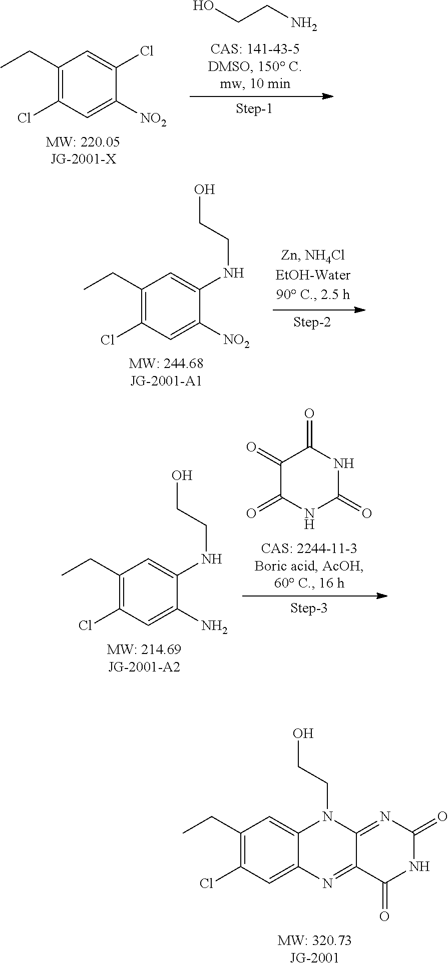

### Step-1: Synthesis of 2-((4-chloro-5-ethyl-2-nitrophenyl)amino)ethan-1-ol

### Batch ID: STL-7-A-525-JG-2001-A1-028-A

To a solution of 1,4-dichloro-2-ethyl-5-nitrobenzene (JG-2001-X) (0.10 g, 0.456 mmol, 1.0 eq) in DMSO (1.5 mL)) 2-aminoethan-1-ol (CAS: 141-43-5) (0.064 g, 1.05 mmol, 5.0 eq) was added and the reaction mixture was heated at 150° C. under microwave irradiation for 10 min. After completion of reaction as indicated by TLC, the reaction mixture was poured into ice cold water (50 mL) and extracted with ethyl acetate (3×50 mL). The combined organic layer was dried over anhydrous sodium sulphate and concentrated under vacuum. The obtained crude product was further purified by column chromatography (35% ethyl acetate/hexane) to obtain title compound as orange solid (0.085 g, 93.27%, 76.44%) LCMS m/z 245.0 & 247.0 (M & M+2).

### Step-2: Synthesis of 2-((2-amino-4-chloro-5-ethylphenyl)amino)ethan-1-ol

### Batch ID: STL-7-A-525-JG-2001-A2-027-A

To a solution of 2-((4-chloro-5-ethyl-2-nitrophenyl)amino)ethan-1-ol (JG-2001-A1) (0.40 g, 1.63 mmol, 1.0 eq) in EtOH:water (9:1 mL), Zn Dust (0.86 g, 13.1 mmol, 8.0 eq) and NH4Cl (0.70 g, 13.1 mmol, 8.0 eq) were added and stirred at 90° C. for 2.5 h. After completion of reaction as indicated by TLC, the reaction mixture was filtered through Celite and filtrate concentrated under reduced pressure the reaction mixture was poured into water (100 mL) and extracted with ethyl acetate (4×25 mL). The combined organic layer was dried over anhydrous sodium sulphate and concentrated under vacuum to afford orange solid as crude (0.37 g, Quantitative) LCMS m/z 215.1 & 217.1 (M & M+2).

### Step-3: Synthesis of 7-chloro-8-ethyl-10-(2-hydroxyethyl)benzo[g]pteridine-2,4(3H, 10H)-dione (JG-2001)

### Batch ID: STL-7-A-525-JG-2001-031-A

To a solution of 2-((2-amino-4-chloro-5-ethylphenyl)amino)ethan-1-ol (JG-2001-A2) (0.300 g, 1.40 mmol, 1.0 eq) in AcOH (6 mL), alloxan monohydrate (CAS No: 2244-11-3) (0.224 g, 1.40 mmol, 1.0 eq) and boric anhydride (0195 g, 2.80 mmol, 2.0 eq) were added and the reaction mixture was stirred at 60° C. for 16 hrs. After completion of reaction as indicated by TLC, the reaction mixture was poured into water (50 mL) and extracted with 10% DCM:MeOH (3×50 mL). The combined organic layer was dried over anhydrous sodium sulphate and concentrated under vacuum. The obtained crude product was further purified by column chromatography (10% MDC:MeOH) to afford orange solid (0.221 g, 49.31%) LCMS m/z 321.2 & 323.2 (M & M+2); 1H NMR (400 MHz, DMSO) δ 11.46 (s, 1H), 8.22 (s, 1H), 8.05 (s, 1H), 4.97 (t, J=5.6 Hz, 1H), 4.71 (t, J=6, 2H), 3.83 (m, 2H), 2.95 (q, J=7.6, 2H), 1.30 (t, J=7.6 Hz, 3H).

### Synthesis of JG-2004

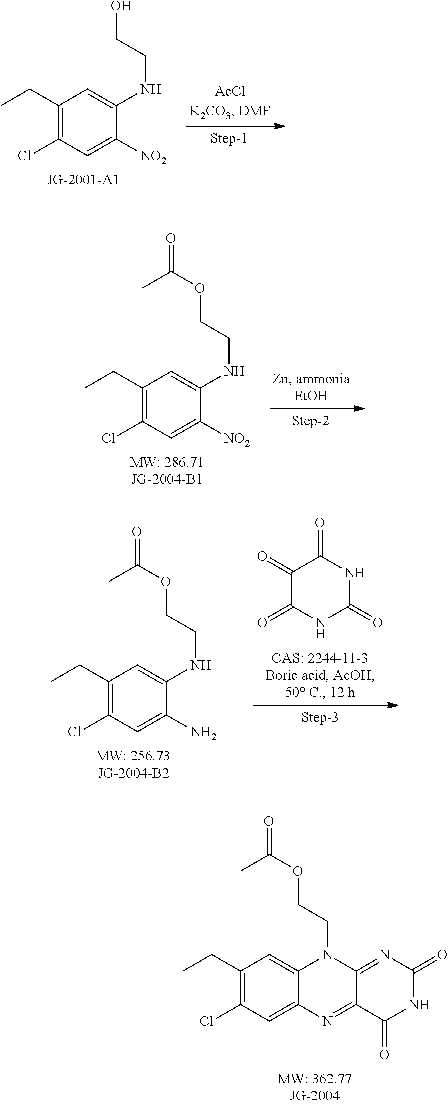 [SMILES: CCc1cc(NCCO)c([N+](=O)[O-])cc1Cl ; CCc1cc(NCCOC(C)=O)c([N+](=O)[O-])cc1Cl ; CCc1cc(NCCOC(C)=O)c(N)cc1Cl ; O=C1NC(=O)C(=O)C(=O)N1 ; CCc1cc2c(cc1Cl)nc1c(=O)[nH]c(=O)nc-1n2CCOC(C)=O]

JG-2001-A1 (150 mg, 1 eq) was combined with acetyl chloride (3 eq) and K2CO3 (2 eq) in DMF, cooled to 0 0 C, then allowed to warm to room temperature for 30 minutes. Product formation was confirmed by LCMS and 1H-NMR and purified by column chromatography (JG-2004-B1, yield 130 mg). Next, JG-2004-B1 (120 mg, 1 eq) was combined with Zn (3 eq) and NH4Cl (5 eq) in a 1:1 mixture of ethanol and water, then incubated at room temperature for 3 hours. Product formation was confirmed by TLC, LCMS and 1H-NMR, then purified by column chromatography (JG-2004-B2, yield 50 mg). JG-2004-B2 (30 mg, 1 eq) was mixed with CA 2244-11-3 (1 eq) and CAS 1303-86-1 (1 eq) in acetic acid, heated to 70° C. for 1 hour. Product formation (JG-2004) was confirmed by TLC, LCMS and purified by column chromatography (yield 10 mg).

2-(7-chloro-8-ethyl-2,4-dioxo-3,4-dihydrobenzo[g]pteridin-10(2H)-yl)ethyl acetate (JG-2004)-1H NMR (400 MHz, DMSO, ppm) δ 11.49 (s, 1H), 8.23 (s, 1H), 8.04 (s, 1H), 4.89 (t, J=5.6 Hz, 2H), 4.42 (t, J=5.2 Hz, 2H), 2.95 (q, J=7.2 Hz, 2H), 1.89 (s, 3H), 1.30 (t, J=7.6 Hz, 3H). LCMS, calc'd 362.77 m/z; found 363.12 m/z

### Synthesis of two methylene linker compounds (JG-2042, JG-2039, JG-2040, JG-2041, JG-2043, JG-2048, JG-2049, JG-2050, JG-2026, JG-2027)

### Synthesis of 7-chloro-8-ethyl-10-(2-(pyridin-3-yl)ethyl)benzo[g]pteridine-2,4(3H, 10H)-dione (JG-2042)

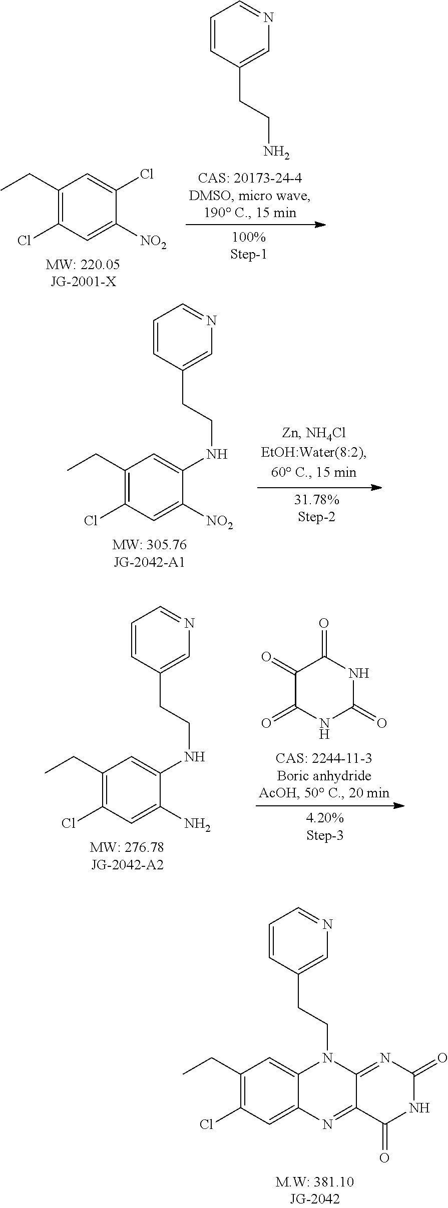 [SMILES: NCCc1cccnc1 ; CCc1cc(Cl)c([N+](=O)[O-])cc1Cl ; CCc1cc(NCCc2cccnc2)c([N+](=O)[O-])cc1Cl ; CCc1cc(NCCc2cccnc2)c(N)cc1Cl ; O=C1NC(=O)C(=O)C(=O)N1 ; CCc1cc2c(cc1Cl)nc1c(=O)[nH]c(=O)nc-1n2CCc1cccnc1]

### Step-1: Synthesis of 4-chloro-5-ethyl-2-nitro-N-(2-(pyridin-3-yl)ethyl)aniline (JG-2042-A1)

### Batch ID: STL-7-A-588-JG-2042-A1-111-a

To a solution of 1,4-dichloro-2-ethyl-5-nitrobenzene (JG-2001-X) (0.200 g, 0.91 mmol, 1 eq) in DMSO (3 mL), 2-(22yridine-3-yl)ethan-1-amine (CAS: 20173-24-4) (0.445 g, 3.65 mmol, 3.0 eq) was added and the reaction mixture was heated at 190° C. under microwave irradiation for 15 min. After completion of reaction as indicated by TLC, the reaction mixture was poured into water (10 mL) and extracted with ethyl acetate (3×10 mL). The combined organic layer was dried over anhydrous sodium sulphate and concentrated under vacuum to afford yellow liquid (0.30 g, 100%) LCMS m/z 306.5 & 308.5 (M & M+2). Note: Crude material was directly used in next step without further purification.

### Step-2: Synthesis of 4-chloro-5-ethyl-N₁-(2-(22 yridine-3-yl)ethyl)benzene-1,2-diamine (JG-2042-A2)

### Batch ID: STL-7-A-588-JG-2042-A2-119-b

To a solution of 4-chloro-5-ethyl-2-nitro-N-(2-(22yridine-3-yl)ethyl)aniline (JG-2042-A1) (0.3 g, 0.98 mmol, 1.0 eq) in EtOH:water (8:2 mL), Zn Dust (0.513 g, 7.80 mmol, 8.0 eq) and NH4Cl (0.419 g, 7.80 mmol, 8.0 eq) were added and stirred at 60° C. for 15 min. After completion of reaction as indicated by TLC, the reaction mixture was filtered through Celite and filtrate was extracted with ethyl acetate (2×10 mL). The combined organic layer was dried over anhydrous sodium sulphate and concentrated under reduced pressure. The obtained crude product was further purified by column chromatography (20% ethyl acetate/hexane) to obtain title compound as yellow solid (0.086 g, 31.78%) LCMS m/z 276.5 & 278.5 (M & M+2).

### Step-3: Synthesis of 7-chloro-8-ethyl-10-(2-(pyridin-3-yl)ethyl)benzo[g]pteridine-2,4(3H, 10H)-dione (JG-2042)

### Batch ID: STL-7-A-588-JG-2042-133-b

To a solution of 4-chloro-5-ethyl-N₁-(2-(pyridin-3-yl)ethyl)benzene-1,2-diamine (JG-2042-A2) (0.086 g, 0.31 mmol, 1.0 eq) in AcOH (1.5 mL), alloxan monohydrate (CAS No: 2244-11-3) (0.049 g, 0.31 mmol, 1.0 eq) and boric anhydride (0.043 g, 0.62 mmol, 2.0 eq) were added and the reaction mixture was stirred at 50° C. for 20 min. After completion of reaction as indicated by TLC, The reaction mixture was slowly poured into ice cooled water (10 mL) and extracted with ethyl acetate (4×10 mL). The combined organic layer was dried over anhydrous sodium sulphate and concentrated under reduced pressure. The obtained crude product was further purified by column chromatography (8.2% MeOH/DCM) to obtain title compound as yellow solid (0.003 g, 4.20%). LCMS m/z 382.7 & 384.7 (M & M+2); 1H NMR (400 MHz, DMSO) δ 11.48 (s, 1H), 8.53 (s, 1H), 8.41 (d, J=4.0 Hz, 1H), 8.18 (s, 1H), 7.77-7.64 (m, 2H), 7.29 (dd, J=7.5, 4.9 Hz, 1H), 4.85 (t, J=6.8 Hz, 2H), 3.10 (t, J=6.9 Hz, 2H), 2.88-2.80 (m, 2H), 1.20 (t, J=7.4 Hz, 3H).

The following compounds were made according to the procedure described for JG-2042 using JG-2001-X.

| Target ID | Structure | Analytical data |
|---|---|---|
| JG-2039 | 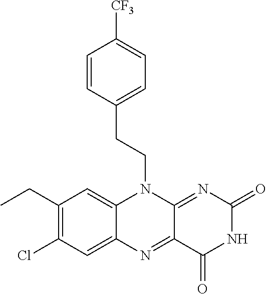 [SMILES: CCc1cc2c(cc1Cl)nc1c(=O)[nH]c(=O)nc-1n2CCc1ccc(C(F)(F)F)cc1] | LCMS m/z 449.8 & 451.8 (M & M + 2); ¹H NMR (400 MHz, DMSO) δ 11.49 (s, 1H), 8.18 (s, 1H), 7.66 (s, 1H), 7.63- 7.54 (m, 4H), 4.86 (bs, 2H), 3.17 (bs, 2H), 2.82 (q, 2H), 1.17 (t, J = 6.8 Hz, 3H). |
| JG-2040 | 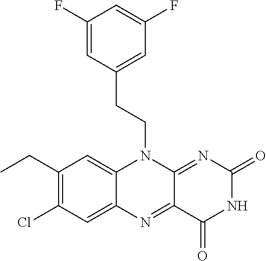 [SMILES: CCc1cc2c(cc1Cl)nc1c(=O)[nH]c(=O)nc-1n2CCc1cc(F)cc(F)c1] | LCMS m/z 417.1 & 419.0 (M & M + 2); 1H NMR (400 MHz, DMSO, ppm) δ 11.49 (s, 1H), 8.19 (s, 1H), 7.70 (s, 1H), 7.09 (m, J = 6.4 Hz, 3H), 4.84 (t, 2H), 3.10 (t, J = 6.4 Hz, 2H), 2.84 (q, J = 7.2 Hz, 2H), 1.21 (t, J = 7.2 Hz, 3H) |
| JG-2041 | 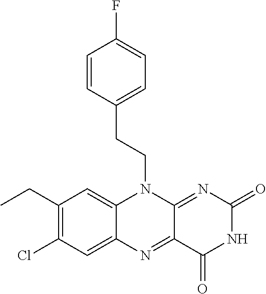 [SMILES: CCc1cc2c(cc1Cl)nc1c(=O)[nH]c(=O)nc-1n2CCc1ccc(F)cc1] | LCMS m/z 399.2 & 401.0 (M & M + 2); 1H NMR (400 MHz, DMSO, ppm) δ 11.47 (s, 1H), 8.18 (s, 1H), 7.69 (s, 1H), 7.36 (q, J = 6 Hz, 2H), 7.10 (t, J = 8.8 Hz, 2H), 4.81 (t, J = 7.2 Hz, 2H), 3.05 (t, J = 6.85 Hz, 2H), 2.85 (q, J = 7.2 Hz, 2H), 1.21 (t, J = 7.6 Hz, 3H) |
| JG-2043 | 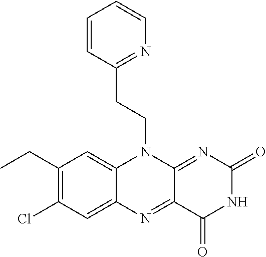 [SMILES: CCc1cc2c(cc1Cl)nc1c(=O)[nH]c(=O)nc-1n2CCc1ccccn1] | LCMS m/z 382.6 & 384.7 (M & M + 2); ¹H NMR (400 MHz, DMSO) δ 11.47 (s, 1H), 8.53 (d, J = 3.6 Hz, 1H), 8.20 (s, 1H), 7.78 (s, 1H), 7.69 (m, 1H), 7.33 (d, J = 7.6 Hz, 1H), 7.25 (m, 1H), 4.98 (t, 2H), 3.24 (t, 2H), 2.87 (q, 2H), 1.24 (t, J = 6.8 Hz, 3H). |
| JG-2048 | 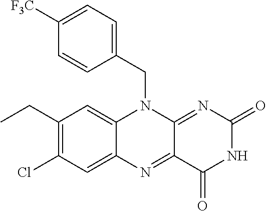 [SMILES: CCc1cc2c(cc1Cl)nc1c(=O)[nH]c(=O)nc-1n2Cc1ccc(C(F)(F)F)cc1] | LCMS m/z 435.1 & 437.1 (M & M + 2); 1H NMR (400 MHz, DMSO, ppm) δ 11.52 (s, 1H), 8.25 (s, 1H), 7.65 (m, J = 8 Hz, 5H), 5.99 (s, 2H), 2.81 (q, J = 7.2 Hz, 2H), 1.13 (t, J = 7.6 Hz, 3H) |
| JG-2049 | 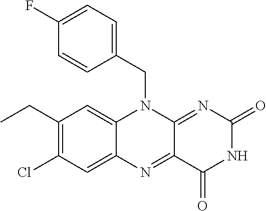 [SMILES: CCc1cc2c(cc1Cl)nc1c(=O)[nH]c(=O)nc-1n2Cc1ccc(F)cc1] | LCMS m/z 385.6 & 387.56 (M & M + 2); 1H NMR (400 MHz, DMSO, ppm) δ 11.50 (s, 1H), 8.23 (s, 1H), 7.6 (s, 1H), 7.46 (d-d, J = 5.6 Hz, 2H), 7.17 (t, J = 8.8 Hz, 2H), 5.89 (s, 2H), 2.81 (q, J = 7.6 Hz, 2H), 1.15 (t, J = 7.6 Hz, 3H) |
| JG-2050 | 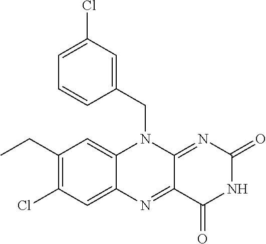 [SMILES: CCc1cc2c(cc1Cl)nc1c(=O)[nH]c(=O)nc-1n2Cc1cccc(Cl)c1] | LCMS m/z 401.2 & 403.1 (M & M + 2); 1H NMR (400 MHz, DMSO, ppm) δ 11.51 (s, 1H), 8.24 (s, 1H), 7.64 (s, 1H), 7.50 (s, 1H), 7.37 (d, J = 5.2 Hz, 3H), 5.90 (s, 2H), 2.81 (q, J = 7.2 Hz, 2H), 1.47 (t, J = 7.6 Hz, 3H) |
| JG-2026 | 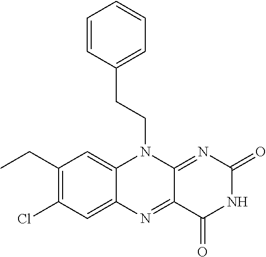 [SMILES: CCc1cc2c(cc1Cl)nc1c(=O)[nH]c(=O)nc-1n2CCc1ccccc1] | LCMS m/z 381.1 & 383.1 (M & M + 2); 1H NMR (400 MHz, DMSO, ppm) δ 11.47 (s, 1H), 8.19 (s, 1H), 7.7 (1H, s), 7.34 (d-d, J = 7.2 Hz, 2H), 7.27 (d-d, J = 7.6 Hz, 2H), 7.22 (m, J = 6.82 Hz, 1H), 4.8 (t, 2H), 3.06 (t, J = 6.8 Hz, 2H), 2.85 (q, J = 7.2 Hz, 2H), 1.23 (t, J = 7.2 Hz, 3H) |
| JG-2027 | 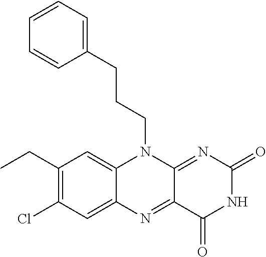 [SMILES: CCc1cc2c(cc1Cl)nc1c(=O)[nH]c(=O)nc-1n2CCCc1ccccc1] | LCMS m/z 385.1 & 397.3 (M & M + 2); 1H NMR (400 MHz, DMSO, ppm) δ 11.44 (s, 1H), 8.21 (s, 1H), 7.67 (s, 1H), 7.29 (d-d, J = 6.8 Hz, 2H), 7.29 (d-d, J = 6.8 Hz, 2H), 7.21 (m, J = 6.4 Hz, 1H), 4.6 (t, 2H), 3.13 (q, J = 7.7 Hz, 2H), 2.90 (m, J = 7.6 Hz, 2H), 2.81 (t, J = 7.6 Hz, 2H), 1.25 (t, J = 7.6 Hz, 3H) |

### Synthesis methylene sidechain derivatives (JG-2046, JG-2047, JG-2036, JG-2038, JG-2045, JG-2028)

### Synthesis of 7-chloro-10-(cyclohexylmethyl)-8-ethylbenzo[g]pteridine-2,4(3H, 10H)-dione (JG-2046)

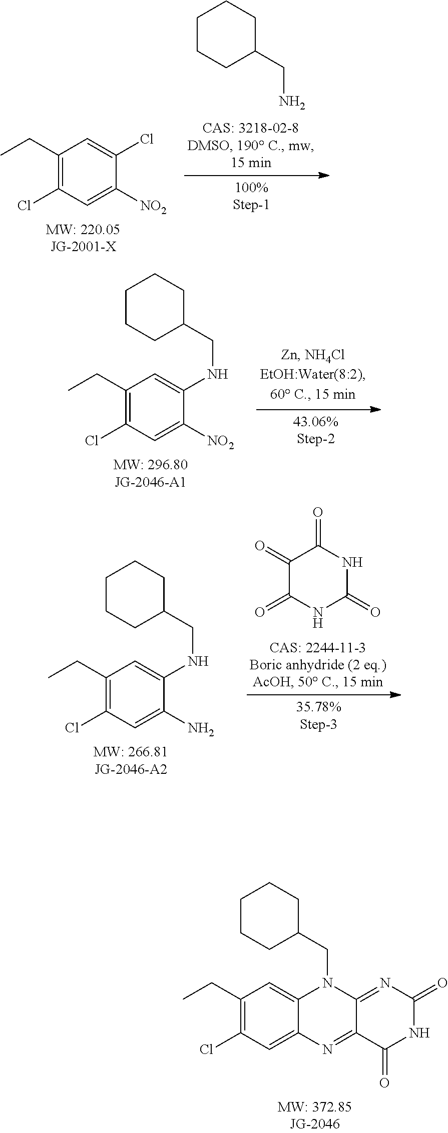 [SMILES: NCC1CCCCC1 ; CCc1cc(Cl)c([N+](=O)[O-])cc1Cl ; CCc1cc(NCC2CCCCC2)c([N+](=O)[O-])cc1Cl ; CCc1cc(NCC2CCCCC2)c(N)cc1Cl ; O=C1NC(=O)C(=O)C(=O)N1 ; CCc1cc2c(cc1Cl)nc1c(=O)[nH]c(=O)nc-1n2CC1CCCCC1]

### Step-1: Synthesis of 4-chloro-N-(cyclohexylmethyl)-5-ethyl-2-nitroaniline

### Batch ID: STL-7-A-588-JG-2046-A1-110-a

To a solution of 1,4-dichloro-2-ethyl-5-nitrobenzene (JG-2001-X) (0.2009 g, 0.91 mmol, 1 eq) in DMSO (3 ml), cyclohexylmethanamine (CAS: 3218-02-8) (0.413 g, 3.62 mmol, 3.0 eq) was added and the reaction mixture was stirred at 190° 0 temperature under microwave irradiation for 15 min. After completion of reaction as indicated by TLC, the reaction mixture was poured into water (10 mL) and extracted with ethyl acetate (3×10 mL). The combined organic layer was dried over anhydrous sodium sulphate and concentrated under vacuum to obtain title compound as yellow solid (0.310 g, 100%). LCMS m/z 297.6 &299.7 (M & M+2). Crude material which was directly used in next step without further purification.

### Step-2: Synthesis of 4-chloro-N1-(cyclohexylmethyl)-5-ethylbenzene-1,2-diamine

### Batch ID: STL-7-A-588-JG-2046-A2-120-b

To a solution of 4-chloro-N-(cyclohexylmethyl)-5-ethyl-2-nitroaniline (JG-2046-A1) (0.310 g, 1.04 mmol, 1.0 eq) in EtOH:water (8:2 mL), Zn Dust (0.546 g, 8.35 mmol, 8.0 eq) and NH4Cl (0.446 g, 8.35 mmol, 8.0 eq) were added and the reaction mixture was stirred at 60° C. for 15 min. After completion of reaction as indicated by TLC, the reaction mixture was filtered through celite and extracted with ethyl acetate (2×10 mL). The combined organic layer was dried over anhydrous sodium sulphate and concentrated under vacuum. The obtained crude product was further purified by column chromatography (30% ethyl acetate/hexane) to obtain yellow solid (0.120 g, 43.06%). LCMS m/z 267.6 & 269.6 (M & M+2).

### Step-3: Synthesis of 7-chloro-10-(cyclohexylmethyl)-8-ethylbenzo[g]pteridine-2,4(3H, 10H)-dione

### Batch ID: STL-7-A-588-JG-2046-134-a

To a solution of 4-chloro-N₁-(cyclohexylmethyl)-5-ethylbenzene-1,2-diamine (JG-2046-A2) (0.120 g, 0.44 mmol, 1.0 eq) in AcOH (1.5 mL), alloxan monohydrate (CAS: 2244-11-3) (0.072 g, 0.44 mmol, 1.0 eq) and Boric anhydride (0.062 g, 0.89 mmol, 2.0 eq) were added and the reaction mixture was stirred at 50° C. for 15 min. After completion of reaction as indicated by TLC, the reaction mixture was poured into water and extracted with ethyl acetate (4×10 mL). The reaction mixture was dried over anhydrous sodium sulphate and concentrated under vacuum. The obtained crude product was further purified by column chromatography (5% MeOH and DCM) to obtain title compound as yellow solid (0.060 g, 35.78%) LCMS m/z 373.8 (M+1); 1H NMR (400 MHz, DMSO) δ 11.42 (s, 1H), δ 8.21 (s, 1H), 7.96 (s, 1H), 4.50 (bs, 2H), 2.95 (q, J=7.2 Hz 2H), 2.10-1.90 (m, 1H), 1.70-1.52 (in, 4H), 1.30 (t, 3H), 1.30-1.00 (in, 6H).

The following compounds were made according to the procedure described for JG-2046 using JG-2001-X.

| Target ID | Structure | Analytical data |
|---|---|---|
| JG-2047 | 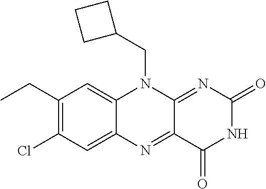 [SMILES: CCc1cc2c(cc1Cl)nc1c(=O)[nH]c(=O)nc-1n2CC1CCC1] | LCMS m/z 345.5 & 347.6 (M & M + 2); 1H NMR (400 MHz, DMSO, ppm) δ 11.43 (s, 1H), 8.21 (s, 1H), 8.0 (s, 1H), 4.72 (d, J = 6.4 Hz, 2H), 2.95 (q, J = 7.2 Hz, 2H), 2.89 (m, J = 7.6 Hz, 1H), 1.98 (m, J = 8.8 Hz, 4H), 1.80 (m, J = 7.6 Hz, 2H), 1.29 (t, J = 7.2 Hz, 3H) |
| JG-2036 | 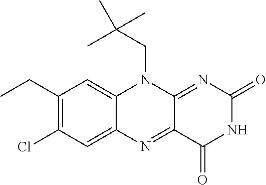 [SMILES: CCc1cc2c(cc1Cl)nc1c(=O)[nH]c(=O)nc-1n2CC(C)(C)C] | LCMS m/z 347.5 & 349.9 (M & M + 2); 1H NMR (400 MHz, DMSO, ppm) δ 11.44 (s, 1H), 8.19 (s, 1H), 8.08 (s, 1H), 4.97 (s, 1H), 4.34 (s, 1H), 2.94 (q, J = 7.2 Hz, 2H), 1.27 (t, J = 7.2 Hz, 3H), 1.00 (s, 9H) |
| JG-2038 | 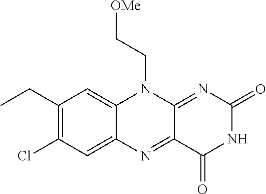 [SMILES: CCc1cc2c(cc1Cl)nc1c(=O)[nH]c(=O)nc-1n2CCOC] | LCMS m/z 335.6 & 337.6 (M & M + 2); 1H NMR (400 MHz, DMSO) δ 11.45 (s, 1H), δ 8.20 (s, 1H), 8.02 (s, 1H), 4.80 (t, 2H), 3.75 (t, 2H), 3.26 (s, 3H), 2.94 (q, J = 7.2 Hz, 2H), 1.29 (t, J = 7.2 Hz, 3H). |
| JG-2045 | 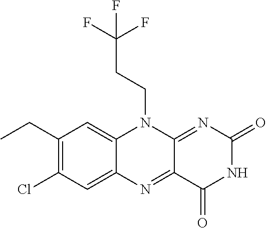 [SMILES: CCc1cc2c(cc1Cl)nc1c(=O)[nH]c(=O)nc-1n2CCC(F)(F)F] | LCMS m/z 373.2 & 375.2 (M & M + 2); 1H NMR (400 MHz, DMSO, ppm) δ 11.52 (s, 1H), 8.25 (s, 1H), 7.90 (s, 1H), 4.85 (t, J = 6.8 Hz, 2H), 2.96 (q, J = 7.2 Hz, 2H), 2.83 (q, J = 7.6 Hz, 2H), 1.35 (t, J = 7.2 Hz, 3H) |
| JG-2028 | 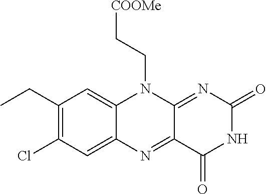 [SMILES: CCc1cc2c(cc1Cl)nc1c(=O)[nH]c(=O)nc-1n2CCC(=O)OC] | LCMS m/z 363.5 & 365.7 (M & M + 2); 1H NMR (400 MHz, DMSO, ppm) δ 11.48 (s, 1H), 8.22 (s, 1H), 8.05 (s, 1H), 4.80 (t, J = 7.6 Hz, 2H), 3.66 (s, 3H), 2.97 (q, J = 7.6 Hz, 2H), 2.82 (t, J = 7.6 Hz, 2H), 1.3 (t, J = 7.6 Hz, 3H) |

### Synthesis of JG-2003

### (10-(2-(benzyloxy)ethyl)-7-chloro-8-ethylbenzo[g]pteridine-2,4(3H, 10H)-dione)

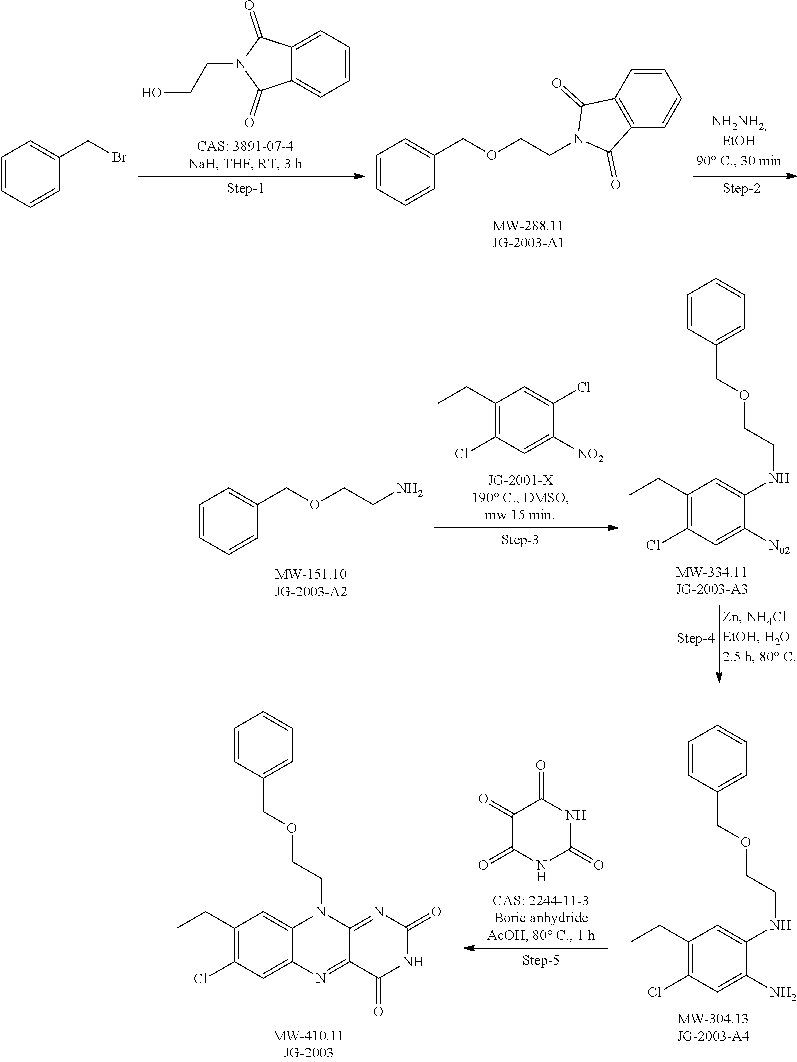

### Step-1: Synthesis of 2-(2-(benzyloxy)ethyl)isoindoline-1,3-dione

### Batch ID: STL-7-A-525-JG-2003-A1-095-A

To a solution of (bromomethyl)benzene (CAS: 100-39-0) (1.0 g, 5.23 mmol, 1 eq) in THE (10 mL), NaH (0.19 g, 7.84 mmol, 1.5 eq) and 2-(2-hydroxyethyl)isoindoline-1,3-dione (0.89 g, 5.23 mmol, 1eq) were added and the reaction mixture was stirred at RT for 3 h. After completion of reaction as indicated by TLC, the reaction mixture was poured into water (50 mL) and extracted with ethyl acetate (3×50 mL). The combined organic layer was dried over anhydrous sodium sulphate and concentrated under vacuum to obtain yellow liquid (1.19 g, 72.40%). The crude was forwarded to next step.

### Step-2: Synthesis of 2-(benzyloxy)ethan-1-amine

### Batch ID: STL-7-A-525-JG-2003-A2-096-Ao

To a solution of 2-(2-(benzyloxy)ethyl)isoindoline-1,3-dione (JG-2003-A1) (1.19 g) in ethanol (17 mL), hydrazine hydrate (1.7 ml) was added and the reaction mixture was stirred at 90° C. for 30 min. After completion of reaction as indicated by TLC, the reaction mixture was filtered and filtrate was concentrated under vacuum. The filtrate was poured into water (50 mL) and extracted with (9:1 MDC: MeOH) (3×50 mL). The combined organic layer was dried over anhydrous sodium sulphate and concentrated under vacuum to afford yellow liquid (0.60 g, 93.80%) LCMS m/z 152.1 (M+1).

### Step-3: Synthesis of N-(2-(benzyloxy)ethyl)-4-chloro-5-ethyl-2-nitroaniline

### Batch ID: STL-7-A-525-JG-2003-A3-098-A

To a solution of 1,4-dichloro-2-ethyl-5-nitrobenzene (JG-2001-X) (0.29 g, 1.31 mmol, 1.0 eq) in DMSO (9 mL), 2-(benzyloxy)ethan-1-amine (JG-2003-A2) (0.600 g, 3.95 mmol, 3.0 eq) was added and the reaction mixture was heated at 190° C. under microwave irradiation for 15 min. After completion of reaction as indicated by TLC, the reaction mixture was poured into ice cold water (50 mL) and extracted with ethyl acetate (3×50 mL). The combined organic layer was dried over anhydrous sodium sulphate and concentrated under vacuum to obtain orange solid. The crude was carry forwarded for next step without any purification. LCMS m/z 335.3 (M+1).

### Step-4: Synthesis of N₁-(2-(benzyloxy)ethyl)-4-chloro-5-ethylbenzene-1,2-diamine

### Batch ID: STL-7-A-525-JG-2003-A4-099-A

To a solution of N-(2-(benzyloxy)ethyl)-4-chloro-5-ethyl-2-nitroaniline (JG-2003-A3) (1.03 g, 3.08 mmol, 1.0 eq) in EtOH:water (10:2.5 mL), Zn Dust (1.60 g, 24.5 mmol, 8.0 eq) and NH4Cl (1.31 g, 25.52 mmol, 8.0 eq) were added and stirred at 80° C. for 2.5 hr. After completion of reaction as indicated by TLC, the reaction mixture was filtered through Celite and filtrate was concentrated under reduced pressure. The filtrate was poured into ice cold water (50 mL) and extracted with ethyl acetate (3×50 mL). The combined organic layer was dried over anhydrous sodium sulphate and concentrated under vacuum. The crude was purified by column chromatography (30% ethyl acetate/hexane) to obtain title compound (0.16 g, 17.15%, over 2 steps). LCMS m/z 304.6 & 306.9 (M & M+2).

### Step-5: Synthesis of 10-(2-(benzyloxy)ethyl)-7-chloro-8-ethylbenzo[g]pteridine-2,4(3H, 10H)-dione (JG-2003)

### Batch ID: STL-7-A-525-JG-2003-117-B

To a solution of N1-(2-(benzyloxy)ethyl)-4-chloro-5-ethylbenzene-1,2-diamine (JG-2003-A4) (0.15 g, 0.493 mmol, 1.0 eq) in AcOH (3 mL), alloxan monohydrate (CAS No: 2244-11-3) (0.078 g, 0.49 mmol, 1.0 eq) and boric anhydride (0.068 g, 0.98 mmol, 2.0 eq) were added and the reaction mixture was stirred at 80° C. for 1 hr. After completion of reaction as indicated by TLC, The reaction mixture was poured into ice cooled water (10 mL) and filtered the solid. The obtained crude product was further triturated with diethylether to obtain title compound as yellow solid (0.065 g, 32.13%). LCMS m/z 411.6 & 413.6 (M & M+2); 1H NMR (400 MHz, DMSO) δ 11.44 (s, 1H), 8.19 (s, 1H), 8.03 (s, 1H), 7.22-7.11 (m, 5H), 4.85 (t, 2H), 4.45 (s, 2H), 3.91 (t, J=4.8 Hz, 2H), 2.87 (q, J=7.2 Hz, 2H), 1.17 (t, J=7.2 Hz, 3H).

### Synthesis of benzyl-oxy-ethyl derivatives (JG-2068, JG-2069, JG-2070, JG-2071, JG-2072, JG-2073, JG-2084, JG-2085, JG-2002-G4)

### Synthesis of 7-chloro-8-ethyl-10-(2-((4-fluorobenzyl)oxy)ethyl)benzo[g]pteridine-2,4(3H, 10H)-dione (JG-2068)

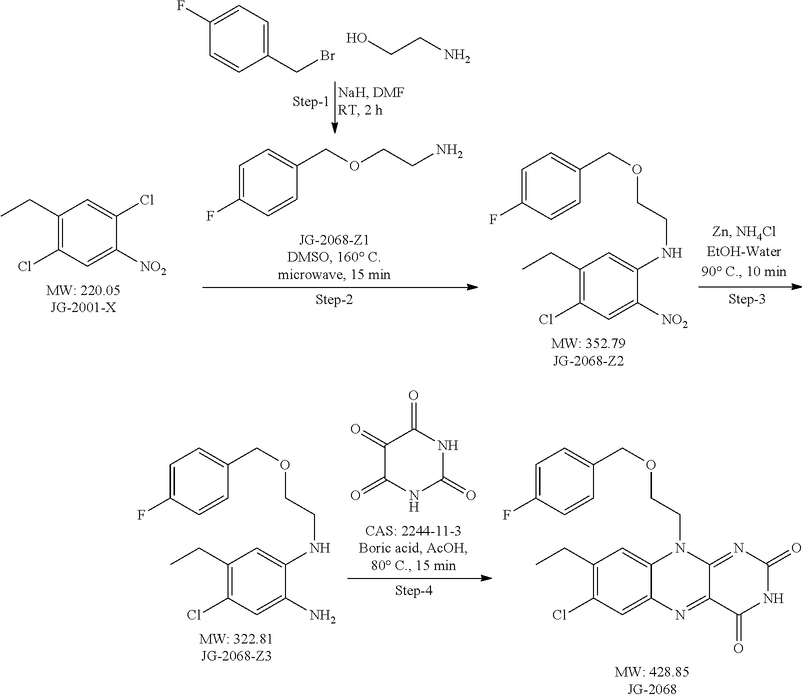

### Step-1: Synthesis of 2-((4-fluorobenzyl)oxy)ethan-1-amine

### Batch ID: STL-7-A-607-JG-2001-Z1-019a

To a solution of 1-(bromomethyl)-4-fluorobenzene (CAS: 459-46-1) (1.0 g, 5.29 mmol, 1 eq) in DMF (5 mL), 2-aminoethan-1-ol (CAS: 141-43-5) (0.645 g, 10.5 mmol, 2.0 eq) and NaH (0.126 g, 5.29 mmol, 1 eq) were added and the reaction mixture was stirred at RT for 2 h. After completion of reaction as indicated by TLC, the reaction mixture was quenched by methanol and poured into water (50 mL) and extracted with ethyl acetate (3×50 mL). The combined organic layer was dried over anhydrous sodium sulphate and concentrated under vacuum to afford yellow liquid. The crude was forwarded to next step. LCMS m/z 170 (M+1).

### Step-2: Synthesis of 4-chloro-5-ethyl-N-(2-((4-fluorobenzyl)oxy)ethyl)-2-nitroaniline

### Batch ID: STL-7-A-588-JG-2001-Z2-183-A

To a solution of 1,4-dichloro-2-ethyl-5-nitrobenzene (JG-2001-X) (0.22 g, 0.99 mmol, 1.0 eq) in DMSO (8 mL), 2-((4-fluorobenzyl)oxy)ethan-1-amine (JG-2001-Z1) (0.84 g, 4.70 mmol, 5.0 eq) was added and the reaction mixture was heated at 160° C. under microwave irradiation for 15 min. After completion of reaction as indicated by TLC, the reaction mixture was poured into ice cold water (50 mL) and extracted with ethyl acetate (3×50 mL). The combined organic layer was dried over anhydrous sodium sulphate and concentrated under vacuum. The obtained crude product was further purified by column chromatography (45% ethyl acetate/hexane) to obtain title compound as yellow solid (0.045 g, 12.93%) LCMS m/z 353.0 & 355.1 (M & M+2).

### Step-3: Synthesis of 4-chloro-5-ethyl-N1-(2-((4-fluorobenzyl)oxy)ethyl)benzene-1,2-diamine

### Batch ID: STL-7-A-607-JG-2001-Z3-029-A

To a solution of 4-chloro-5-ethyl-N-(2-((4-fluorobenzyl)oxy)ethyl)-2-nitroaniline (JG-2001-Z2) (0.29 g, 0.82 mmol, 1.0 eq) in EtOH:water (4:0.5 mL), Zn Dust (0.43 g, 6.57 mmol, 8.0 eq) and NH4Cl (0.35 g, 6.57 mmol, 8.0 eq) were added and stirred at 90° C. for 10 min. After completion of reaction as indicated by TLC, the reaction mixture was filtered through Celite and filtrate was concentrated under reduced pressure. The obtained crude product was further purified by column chromatography (50% ethyl acetate/hexane) to obtain title compound as yellow solid (0.19 g, 71.60%). Mass Ms 323.54 & 325.54 (M+1).

### Step-4: Synthesis of 7-chloro-8-ethyl-10-(2-((4-fluorobenzyl)oxy)ethyl)benzo[g]pteridine-2,4(3H, 10H)-dione

### Batch ID: STL-7-A-607-JG-2068-035-c

To a solution of 4-chloro-5-ethyl-N1-(2-((4-fluorobenzyl)oxy)ethyl)benzene-1,2-diamine (JG-2001-Z3) (0.19 g, 0.59 mmol, 1.0 eq) in AcOH (3 mL), alloxan monohydrate (CAS No: 2244-11-3) (0.094 g, 0.11 mmol, 1.0 eq) and boric anhydride (0.082 g, 0.11 mmol, 2.0 eq) were added and the reaction mixture was stirred at 80° C. for 15 min. After completion of reaction as indicated by TLC, The reaction mixture was poured into ice cooled water (10 mL) and filtered. The obtained crude product was further triturated with diethyl ether to obtain title compound as yellow solid (0.020 g, 7.92%). LCMS m/z 429.0 & 431.1 (M & M+2); 1H NMR (400 MHz, DMSO) δ 11.44 (s, 1H), 8.18 (s, 1H), 8.00 (s, 1H), 7.20-7.12 (m, 2H), 7.06-7.02 (m, 2H), 4.84 (bs, 2H), 4.43 (s, 2H), 3.89 (bs, 2H), 2.84 (q, J=7.2, 2H), 1.14 (t, J=7.2 Hz, 3H).

The following compounds were made according to the procedure described for JG-2068 using JG-2001-X.

| Target ID | Structure | Analytical data |
|---|---|---|
| JG-2069 | 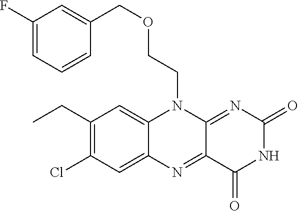 [SMILES: CCc1cc2c(cc1Cl)nc1c(=O)[nH]c(=O)nc-1n2CCOCc1cccc(F)c1] | LCMS m/z 429.0 & 431.0 (M & M + 2); ¹H NMR (400 MHz, DMSO) δ 11.44 (s, 1H), 8.19 (s, 1H), 8.04 (s, 1H), 7.29-7.23 (m, 1H), 7.05-6.95 (m, 2H), 6.87 (d, J = 10 Hz,1H), 4.88-485 (m, 2H), 4.46 (s, 2H), 3.93-3.91 (m, 2H), 2.86 (q, J = 7.6 Hz, 2H), 1.16 (t J = 7.6 Hz 3H). |
| JG-2070 | 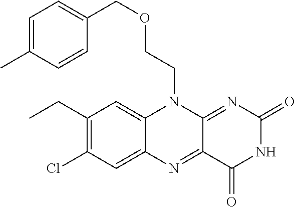 [SMILES: CCc1cc2c(cc1Cl)nc1c(=O)[nH]c(=O)nc-1n2CCOCc1ccc(C)cc1] | LCMS m/z 425.2 & 427.1 (M & M + 2); ¹H NMR (400 MHz, DMSO) δ 11.43 (s, 1H), 8.18 (s, 1H), 7.99 (s, 1H), 6.98 (d, J = 8 Hz, 2H), 6.94 (d, J = 8 Hz, 2H), 4.82 (t, J = 4.8, 2H), 4.37 (s, 2H), 3.87 (t, J = 4.8, 2H), 2.86 (t, J = 7.6, 2H), 2.22 (s, 3H), 1.18 (t, J = 7.2 Hz, 3H). |
| JG-2071 | 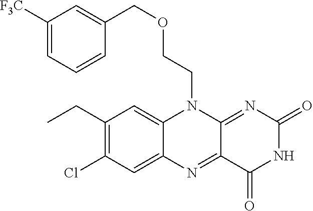 [SMILES: CCc1cc2c(cc1Cl)nc1c(=O)[nH]c(=O)nc-1n2CCOCc1cccc(C(F)(F)F)c1] | LCMS m/z 479.1 & 481 (M & M + 2); ¹H NMR (400 MHz, DMSO) δ 11.44 (s, 1H), 8.18 (s, 1H), 8.03 (s, 1H), 7.56 (d, J =7.2 Hz, 1H), 7.48-7.41 (m, 2H), 7.36 (s, 1H), 4.89 (t, J = 4.8, 2H), 4.54 (s, 2H), 3.96 (t, J = 4.8, 2H), 2.81 (q, J = 7.2 Hz, 2H), 1.09 (t, J = 7.2 Hz, 3H). |
| JG-2072 | 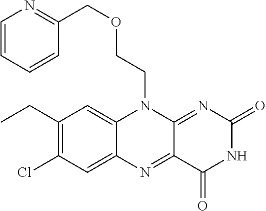 [SMILES: CCc1cc2c(cc1Cl)nc1c(=O)[nH]c(=O)nc-1n2CCOCc1ccccn1] | LCMS m/z 412.0 & 414.0 (M & M + 2); 1H NMR (400 MHz, DMSO) δ 11.45 (s, 1H) 8.43-8.42 (m, 1H), 8.35 (d, 1H), 8.18 (s, 1H), 8.02 (s, 1H), 7.51 (d, J = 7.6 Hz, 1H), 7.25 (dd, J = 4.8 & 8 Hz, 1H), 4.86 (t, 2H), 4.49 (s, 2H), 3.93 (t, 2H), 2.84 (q, J = 7.2 Hz, 2H), 1.13 (t, J = 7 .2 Hz, 3H). |
| JG-2073 | 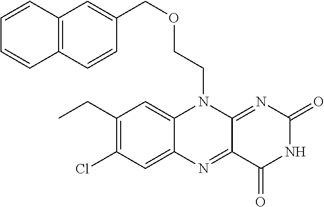 [SMILES: CCc1cc2c(cc1Cl)nc1c(=O)[nH]c(=O)nc-1n2CCOCc1ccc2ccccc2c1] | LCMS m/z 461.1 & 463.1 (M & M + 2); ¹H NMR (400 MHz, DMSO) δ 11.43 (s, 1H), 8.17 (s, 1H), 8.03 (s, 1H), 7.86-7.70 (m, 3H), 7.58 (s, 1H), 7.50- 7.46 (m, 2H), 7.22 (d, J = 8.4 Hz, 1H), 4.89 (t, 2H), 4.61 (s, 2H), 3.96 (t, J = 4.8, 2H), 2.79 (q, J = 7.2 Hz, 2H), 1.09 (t, J = 7.6 Hz, 3H). |
| JG-2084 | 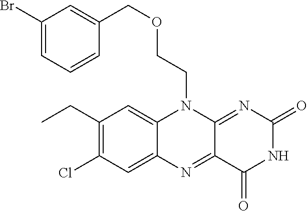 [SMILES: CCc1cc2c(cc1Cl)nc1c(=O)[nH]c(=O)nc-1n2CCOCc1cccc(Br)c1] | LCMS m/z 488.9 & 490.9 (M & M + 2); ¹HNMR (400 MHz, DMSO) δ 11.44 (s, 1H), 8.19 (s, 1H), 8.03 (s, 1H), 7.39 (d, J = 7.6 Hz, 1H), 7.20-7.10 (m, 3H), 4.87 (t, 2H), 4.44 (s, 2H), 3.93 (t, 2H), 2.87 (q, J = 6.8, 2H), 1.15 (t, J = 7.2 Hz, 3H). |
| JG-2085 | 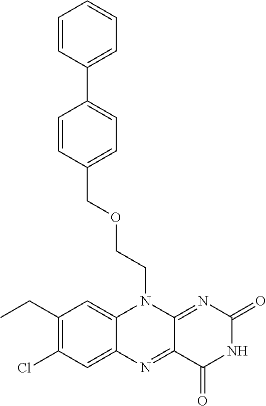 [SMILES: CCc1cc2c(cc1Cl)nc1c(=O)[nH]c(=O)nc-1n2CCOCc1ccc(-c2ccccc2)cc1] | LCMS m/z 487.1 & 489.3 (M & M + 2); ¹H NMR (400 MHz, DMSO) δ 11.44 (s, 1H), 8.18 (s, 1H), 8.03 (s, 1H), 7.61 (d, J = 7.2 Hz, 2H), 7.51-7.35 (m, 5H), 7.19 (d, J = 8.0 Hz, 2H), 4.87 (m, 2H), 4.49 (. 2H), 3.93 (m, 2H), 2.85 (q, J = 7.2 Hz, 2H), 1.15 (t, J = 7.6 Hz, 3H). |
| JG-2002-G4 | 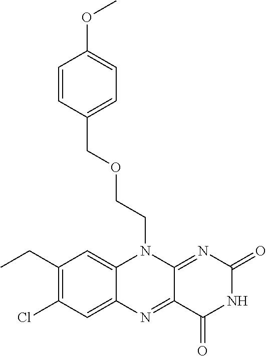 [SMILES: CCc1cc2c(cc1Cl)nc1c(=O)[nH]c(=O)nc-1n2CCOCc1ccc(OC)cc1] | LCMS m/z 441.8 & 443.9 (M & M + 2); ¹H NMR (400 MHz, DMSO) δ 11.44 (s, 1H), 8.19 (s, 1H), 7.98 (s, 1H), 7.02 (d, J = 8.4 Hz, 2H), 6.74 (d,J = 8.4 Hz, 2H), 4.81 (m, 2H), 4.36 (bs, 2H), 3.86 (bs, 2H), 3.70 (s, 3H), 2.85 (q, 2H), 1.17 (t, 3H). |

### Synthesis of alkyl ether analogues (JG-2005, JG-2006, JG-2008, JG-2009, JG-2014, JG-2015, JG-2016, JG-2092)

### Synthesis of 7-chloro-10-(2-(cyclohexylmethoxy)ethyl)-8-ethylbenzo[g]pteridine-2,4(3H, 10H)-dione (JG-2005)

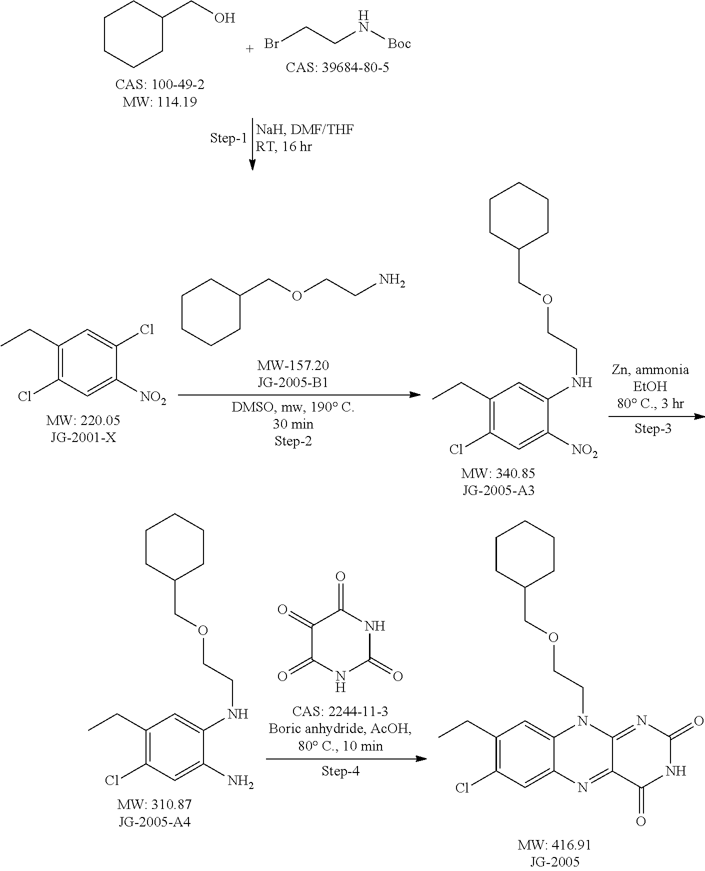

### Step-1: Synthesis of 2-(cyclohexylmethoxy)ethan-1-amine

### Batch ID: STL-7-A-525-JG-2005-B1-188-A

To a solution of cyclohexylmethanol (CAS: 100-49-2) (1.0 g, 8.75 mmol, 1 eq) in THE (10 mL), tert-butyl (2-bromoethyl)carbamate (CAS: 39684-80-5) (1.96 g, 8.75 mmol, 1 eq) and NaH (1.05 g, 43.7 mmol, 5 eq) were added and the reaction mixture was stirred at RT for 16 hr. After completion of reaction as indicated by TLC, the reaction mixture was quenched by methanol and concentrated under vacuum the reaction mixture was poured into water (50 mL) and extracted with (9:1 MDC in MeOH) (3×50 mL). The combined organic layer was dried over anhydrous sodium sulphate and concentrated under vacuum to to afford yellow liquid (1.68 g, crude) 158.4 (M+1). Note: TLC and analysis shown that Boc group was cleaved in reaction itself.

### Step-2: Synthesis of 4-chloro-N-(2-(cyclohexylmethoxy)ethyl)-5-ethyl-2-nitroaniline

### Batch ID: STL-7-A-525-JG-2005-A3-192-A

To a solution of 1,4-dichloro-2-ethyl-5-nitrobenzene (JG-2001-X) (0.300 g, 1.36 mmol, 1.0 eq) in DMSO (2 mL), 2-(cyclohexylmethoxy)ethan-1-amine (JG-2005-B1) (0.645 g, 4.10 mmol, 3.0 eq) was added and the reaction mixture was heated at 190° C. under microwave irradiation for 30 min. After completion of reaction as indicated by TLC, the reaction mixture was poured into ice cold water (50 mL) and extracted with ethyl acetate (3×50 mL). The combined organic layer was dried over anhydrous sodium sulphate and concentrated under vacuum to The obtained crude product was further purified by column chromatography (neat hexane) to afford orange solid (0.057 g, 12.27%) LCMS m/z 341.7 & 343.7 & (M&M+2).

### Step-3: Synthesis of 4-chloro-N1-(2-(cyclohexylmethoxy)ethyl)-5-ethylbenzene-1,2-diamine

### Batch ID: STL-7-A-525-JG-2005-A4-200-A

To a solution of 4-chloro-N-(2-(cyclohexylmethoxy)ethyl)-5-ethyl-2-nitroaniline (JG-2005-A3) (0.215 g, 0.63 mmol, 1.0 eq) in EtOH:water (5:0.8 mL), Zn Dust (0.330 g, 5.05 mmol, 8.0 eq) and NH4Cl (0.270 g, 5.05 mmol, 8.0 eq) were added and stirred at 80° C. for 3 hr. After completion of reaction as indicated by TLC, the reaction mixture was filtered through Celite and filtrate concentrated under reduced pressure. The obtained crude product was further purified by column chromatography (35% ethyl acetate/hexane) to obtain title compound as gray liquid (0.054 g, 27.54%) LCMS m/z 311.6 & 313.6 (M & M+2).

### Step-4: Synthesis of 7-chloro-10-(2-(cyclohexylmethoxy)ethyl)-8-ethylbenzo[g]pteridine-2,4(3H, 10H)-dione

### Batch ID: STL-7-A-588-JG-2005-001-B

To a solution of 4-chloro-N1-(2-(cyclohexylmethoxy)ethyl)-5-ethylbenzene-1,2-diamine (JG-2005-A4) (0.054, 0.17 mmol, 1.0 eq) in AcOH (1.5 mL), alloxan monohydrate (CAS No: 2244-11-3) (0.027 g, 0.17 mmol, 1.0 eq) and boric anhydride (0.024 g, 0.34 mmol, 2.0 eq) were added and the reaction mixture was stirred at 80° C. for 10 min. After completion of reaction as indicated by TLC, the reaction mixture was poured into ice cold water (50 mL) and extracted with ethyl acetate (3×50 mL). The combined organic layer was dried over anhydrous sodium sulphate and concentrated under vacuum. The obtained crude product was further purified by column chromatography (97% ethyl acetate/hexane) to afford yellow solid (0.007 g, 13.81%). LCMS m/z 417.3 & 419.2 (M&M+2); 1HNMR (400 MHz, DMSO) δ 11.44 (s, 1H), 8.20 (s, 1H), 8.04 (s, 1H), 4.81 (t, 2H), 3.78 (t, 2H), 3.15 (d, J=6.0, 2H), 2.92 (q, J=7.6, 2H), 1.56-1.46 (m, 5H), 1.29-0.98 (m, 7H), 0.77-72 (m, 2H).

### Synthesis of 7-chloro-8-ethyl-10-(2-(neopentyloxy)ethyl)benzo[g]pteridine-2,4(3H, 10H)-dione (JG-2014)

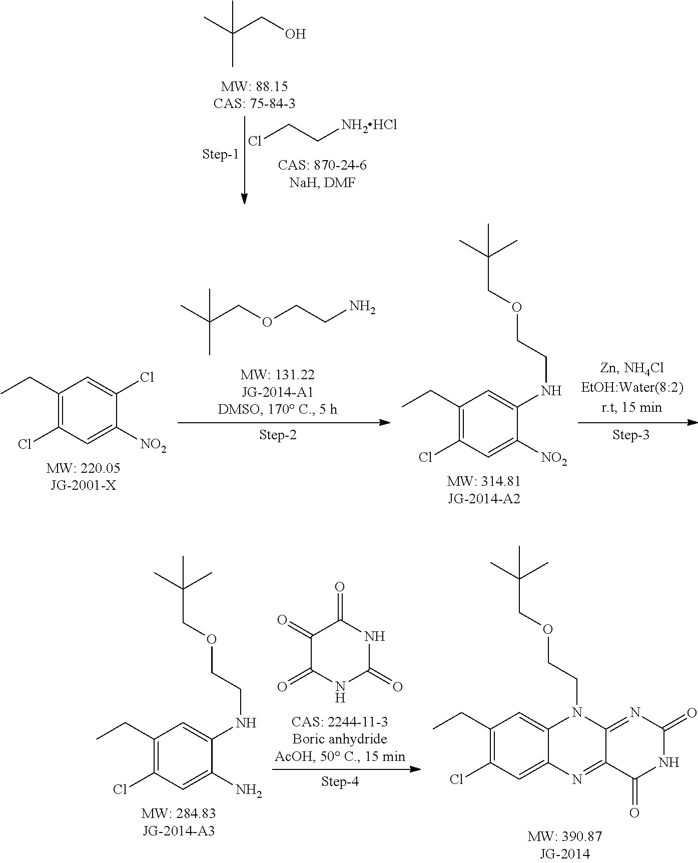

### Step-1: Synthesis of 2-(neopentyloxy)ethan-1-amine (JG-2014-A1)

### Batch ID: STL-9-A708-JG-2014-A1-036

To a solution of 2,2-Dimethyl-1-propanol (CAS: 75-84-3) (10 g, 113.44 mmol, 1.0 eq) in DMF (100 mL/10V), 60% NaH (22.68 g, 567.2 mmol, 5.0 eq) was added portion wise. The reaction mixture was stirred at 0° C. temperature for 1.5 h under nitrogen atmosphere. 2-Chloroethylamine hydrochloride (CAS: 870-24-6) (19.73 g, 170.16 mmol, 1.5 eq) was added portion wise to the reaction mixture. The reaction mixture was allowed to stir for 16 hr at room temperature. After completion of reaction as indicated by TLC (mobile phase: 10% MeOH in DCM), the reaction mixture was slowly dumped in to cold aqueous sodium chloride solution (200 mL). The reaction mixture was extracted with ethyl acetate (3×1.5 L) and combined organic layer was washed with water (2×500 mL) and dried over anhydrous sodium sulphate. The organic layer was dried over anhydrous sodium sulphate and concentrated under vacuum to afford yellow bi-phasic liquid (12 g, 80.61%) which was used in next step without further purification. LCMS: 1.186 min, MS: ES+132.34 (M+1).

### Step-2: Synthesis of 4-chloro-5-ethyl-N-(2-(neopentyloxy)ethyl)-2-nitroaniline (JG-2014-A2)

### Batch ID: STL-9-A708-JG-2014-A2-069

To a solution of 1,4-dichloro-2-ethyl-5-nitrobenzene (JG-2001-X) (1.0 g, 4.54 mmol, 1.0 eq) in DMSO (10 mL/10V) was added 2-(neopentyloxy)ethan-1-amine (JG-2014-A1) (2.98 g, 22.70 mmol, 5.0 eq). The reaction mixture was stirred at 190° C. temperature under microwave for 30 to 45 min. After completion of reaction as indicated by TLC, the reaction mixture was slowly dumped in ice cooled water (300 mL) and extracted with ethyl acetate (3×100 mL). The reaction mixture was dried over anhydrous sodium sulphate and concentrated under vacuum. The obtained crude product was further purified by column chromatography (0.4% ethyl acetate/hexane) to give title compound as yellow solid (0.25 g, 17.47%). Note: Above reaction was repeated in 1 g parallel batch under microwave irradiation. LCMS: 315.3, 317.3 (M & M+2); 1H NMR (400 MHz, DMSO) δ 8.25 (t, J=4.8 Hz, 1H), 8.01 (s, 1H), 7.07 (s, 1H), 3.65-3.63 (m, 2H), 3.56-3.52 (m, 2H), 3.10 (s, 2H), 2.67 (q, J=7.4 Hz, 2H), 1.18 (t, 3H), 0.85 (s, 9H).

### Step-3: Synthesis of 4-chloro-5-ethyl-N1-(2-(neopentyloxy)ethyl)benzene-1,2-diamine (JG-2014-A3)

### Batch ID: STL-9-A708-JG-2014-A3-073a

To a solution of 4-chloro-5-ethyl-N-(2-(neopentyloxy)ethyl)-2-nitroaniline (JG-2014-A2) (2.3 g, 7.46 mol, 1.0 eq) in ethanol:water (10 mL, 8:2 V) were added Zn Dust (3.90 g, 59.7 mmol, 8.0 eq) and NH4Cl (3.19 g, 59.7 mmol, 8.0 eq). The reaction mixture was stirred at room temperature for 15 min. After completion of reaction as indicated by TLC, the reaction mixture was filtered through Celite and extracted with ethyl acetate (2×50 mL). The filtrate was dried over anhydrous sodium sulphate and concentrated under reduced pressure to afford yellow liquid (2.0 g, 96.11%). It was directly used in next step without further purification. LCMS: 285.1, 286.9 (M & M+2).

### Step-4: Synthesis of 7-chloro-8-ethyl-10-(2-(neopentyloxy)ethyl)benzo[g]pteridine-2,4(3H, 10H)-dione (JG-2014)

### Batch ID: STL-9-A708-JG-2014-103-b

To a solution of 4-chloro-5-ethyl-N₁-(2-(neopentyloxy)ethyl)benzene-1,2-diamine (JG-2014-A3) (1.17 g, 4.10 mmol, 1.0 eq) in AcOH (6 mL), alloxan monohydrate (CAS: 2244-11-3) (0.65 g, 4.10 mmol, 1.0 eq) and boric anhydride (0.571 g, 8.20 mmol, 2.0 eq) were added. The reaction mixture was stirred at 50° C. temperature for 15 min. TLC indicated completion of reaction. The reaction mixture was slowly poured into ice cooled water (50 mL) and extracted with ethyl acetate (3×50 mL). The combined organic layer was dried over anhydrous sodium sulphate and concentrated under reduce pressure. The crude was purified by column chromatography (70% ethyl acetate/hexane) to afford yellow solid (0.7 g, 42.60%). LCMS: 391.1, 393.1 (M& M+2). 1H NMR (400 MHz, DMSO) δ 11.43 (s, 1H), 8.19 (s, 1H), 8.05 (s, 1H), 4.83 (bs, 2H), 3.81 (bs, 2H), 3.00 (s, 2H), 2.92 (q, J=7.6 Hz, 2H), 1.29 (t, J=7.4 Hz, 3H), 0.67 (s, 9H).

### Synthesis of 7-chloro-8-ethyl-10-(2-isobutoxyethyl)benzo[g]pteridine-2,4(3H, 10H)-dione (JG-2016)

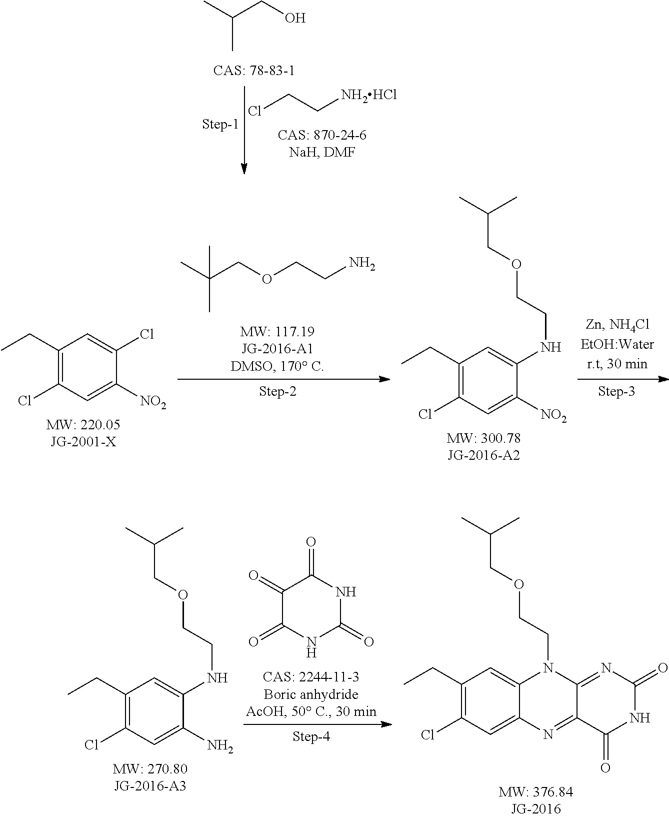

### Step-1: Synthesis of 2-isobutoxyethan-1-amine (JG-2016-A1)

### Batch ID: STL-9-A708-JG-2016-A1-010

To a solution of 2-methylpropan-1-ol (CAS: 78-83-1) (25 g, 337.2 mmol, 1 eq) in DMF (250 mL/10V), 60% NaH (67.45 g, 1686.4 mmol, 5.0 eq) was added portion wise and stirred at 0° C. temperature for 1 h under nitrogen atmosphere. 2-chloroethan-1-amine hydrochloride (CAS: 870-24-6) (58.68 g, 505.9 mmol, 1.5 eq) was added portion wise to the reaction mixture and stirred at room temperature for 4 h. After completion of reaction as indicated by TLC, the reaction mixture was poured into aqueous sodium chloride solution and extracted with ethyl acetate (3×2 L). The combined organic layer was dried over anhydrous sodium sulphate and concentrated under vacuum up to 300 ml then washed with water (2×500 mL). The organic layer was dried over anhydrous sodium sulphate and concentrated under vacuum to afford yellow biphasic liquid (21.48 g, 54.34%) crude material which was directly used in next step without further purification. LCMS: 118.2 (M+1).

### Step-2: Synthesis of 4-chloro-5-ethyl-N-(2-isobutoxyethyl)-2-nitroaniline (JG-2016-A2)

### Batch ID: STL-9-A708-JG-2016-A2-026

To a solution of 1,4-dichloro-2-ethyl-5-nitrobenzene (JG-2001-X) (5.0 g, 22.72 mmol, 1.0 eq) in DMSO (5 mL), 2-isobutoxyethan-1-amine (JG-2016-A1) (13.31 g, 113.6 mmol, 5.0 eq) was added and stirred at 170° C. temperature for 5 h. After completion of reaction as indicated by TLC, the reaction mixture was poured into ice cooled water (300 mL) and extracted with ethyl acetate (3×100 mL). The combined organic layer was dried over anhydrous sodium sulphate and concentrated under vacuum. The crude was further purified by column chromatography (4% ethyl acetate/hexane) to afford yellow liquid (5.0 g, 73.16%) LCMS: 301.3, 303.3 (M & M+2).

### Step-3: Synthesis of 4-chloro-5-ethyl-N₁-(2-isobutoxyethyl)benzene-1,2-diamine (JG-2016-A3)

### Batch ID: STL-9-A708-JG-2016-A3-037

To a solution of 4-chloro-5-ethyl-N-(2-isobutoxyethyl)-2-nitroaniline (JG-2016-A2) (6.1 g, 20.28 mmol, 1.0 eq) in ethanol:water (8:2v) Zn Dust (10.60 g, 162.2 mmol, 8.0 eq) and NH4Cl (8.67 g, 162.2 mmol, 8.0 eq) were added and stirred the reaction mixture at room temperature for 30 min at room temperature. TLC indicated completion of reaction. The reaction mixture was frittered through celite and filtrate was extracted with ethyl acetate (2×50 mL). The organic layer was dried over anhydrous sodium sulphate and concentrated under reduce pressure to afford yellow liquid (5.24 g, 95.41%). The crude product was used in next step without further purification. LCMS: 271.3, 273.2 (M&M+2).

### Step-4: Synthesis of 7-chloro-8-ethyl-10-(2-isobutoxyethyl)benzo[g]pteridine-2,4(3H, 10H)-dione (JG-2016)

### Batch ID: STL-9-A708-JG-2016-038-e

To a solution of 4-chloro-5-ethyl-N1-(2-isobutoxyethyl)benzene-1,2-diamine (JG-2016-A3) (5.24 g, 19.35 mmol, 1.0 eq) in AcOH (25 mL) alloxan monohydrate (CAS: 2244-11-3) (3.09 g, 19.35 mmol, 1.0 eq) and boric anhydride (2.69 g, 38.70 mmol, 2.0 eq) were added and stirred at 50° C. temperature for 30 min. The reaction mixture was poured into ice cooled water (100 mL) and extracted with ethyl acetate (3×50 mL). The reaction mixture was dried over anhydrous sodium sulphate Filtrate was concentrated under reduce pressure. The obtained crude product was further purified by column chromatography (80% ethyl acetate/hexane) and pure fraction was concentrated under vacuum to afford yellow solid (2.8 g, 38.40%). LCMS: 377.3, 379.3 (M&M+2). 1H NMR (400 MHz, DMSO) δ 11.44 (s, 1H), 8.18 (s, 1H), 8.03 (s, 1H), 4.80 (t, 2H), 3.78 (t, J=4.8 Hz, 2H), 3.12 (d, J=6.4 Hz, 2H), 2.91 (q, J=7.2 Hz, 2H), 1.62 (m, 1H), 1.27 (t, J=7.4 Hz, 3H), 0.70 (d, J=6.7 Hz, 6H).

### Synthesis of tert-butyl 4-((2-(7-chloro-8-ethyl-2,4-dioxo-3,4-dihydrobenzo [g]pteridin-10 (2H)-yl) ethoxy) methyl) piperidine-1-carboxylate (JG-2009)

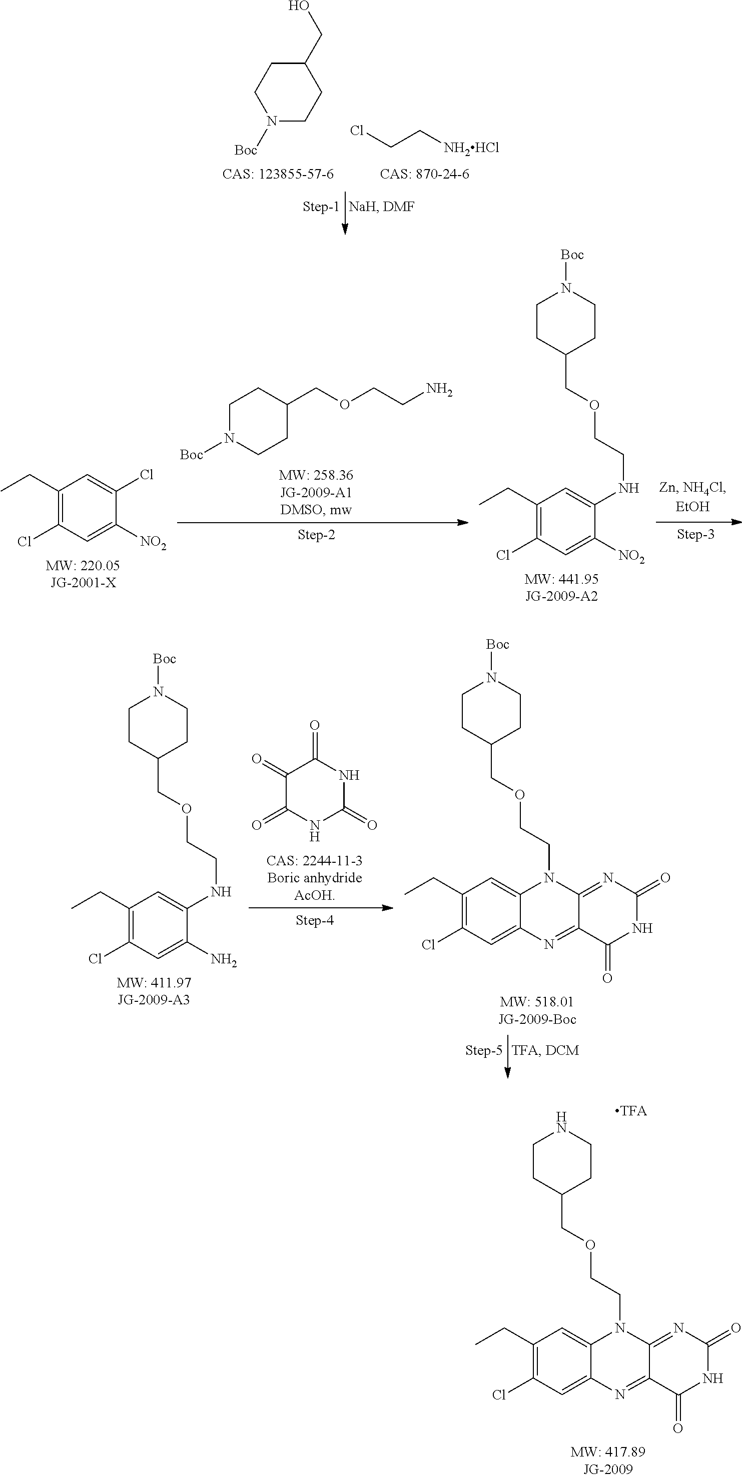

### Step-1: Synthesis of tert-butyl 4-((2-aminoethoxy)methyl)piperidine-1-carboxylate

### Batch ID: STL-7-A-574-JG-2009-A1-139

To a solution of tert-butyl 4-(hydroxymethyl)piperidine-1-carboxylate (CAS: 123855-57-6) (0.5 g, 2.30 mmole, 1 eq) in DMF (20 mL), Sodium hydride (0.93 g, 23.20 mmol, 10.0 eq) was added portion wised at 0° C. and the reaction mixture was allowed to stir for 1 hr at 0° C. 2-chloroethan-1-amine hydrochloride (CAS: 870-24-6) (0.54 g, 4.60 mmole, 2eq) was added slowly at 0° C. and reaction mixture was further stirred at 0° to room temperature for 16 hr. After completion of reaction as indicated by TLC, the reaction mixture was quenched into ice cold water (20 mL) and extracted with ethyl acetate (3×20 mL). The combined organic layer was dried over anhydrous sodium sulphate and concentrated under vacuum to afford crude colourless liquid (0.93 g, quantitative). Reaction was only monitored by TLC and used immediately in next step.

### Step-2: Synthesis of tert-butyl 4-((2-((4-chloro-5-ethyl-2-nitrophenyl)amino)ethoxy)methyl)piperidine-1-carboxylate

### Batch ID: STL-7-A-574-JG-2009-A2-148-B

To a solution of 1,4-dichloro-2-ethyl-5-nitrobenzene (JG-2001-X) (0.25 g, 1.10 mmole, 1 eq) in DMSO (4.0 mL), 4-((2-aminoethoxy)methyl)piperidine-1-carboxylate (JG-2009-A1) (0.9 g, 4.50 mmol, 4.0 eq) was added and heated at 190° C. under microwave irradiation for 7 min. After completion of reaction as indicated by TLC, the reaction mixture was poured into water (10 mL) and extracted with ethyl acetate (3×10 mL). The combined organic layer was dried over anhydrous sodium sulphate and concentrated under vacuum to afford yellow liquid (0.27 g, quantitative), LCMS m/z 442.01. Note: Crude material was directly used in next step without further purification.

### Step-3: Synthesis of tert-butyl 4-((2-((2-amino-4-chloro-5-ethylphenyl) amino) ethoxy) methyl) piperidine-1-carboxylate

### Batch ID: STL-7-A-574-JG-2009-A3-152-b

To a solution of tert-butyl 4-((2-((4-chloro-5-ethyl-2-nitrophenyl)amino)ethoxy)methyl)piperidine-1-carboxylate (JG-2009-A2) (0.27 g, 6.10 mmole, 1.0 eq) in EtOH:water (8:2 mL), Zn Dust (0.261 g, 4.80 mmole, 8.0 eq) and NH4Cl (0.32 g, 4.80 mmole, 8.0eq) were added and stirred at 50° C. for 1 hr. After completion of reaction as indicated by TLC, the reaction mixture was filtered through Celite and filtrate was extracted with ethyl acetate (2×50 mL). The combined organic layer was dried over anhydrous sodium sulphate and concentrated under reduced pressure to obtain crude as sticky solid (0.24 g, quantitative), LCMS m/z 412.2 & 414.2 (M & M+2). Note: Crude material was directly used in next step without further purification.

### Step-4: Synthesis of tert-butyl 4-((2-(7-chloro-8-ethyl-2,4-dioxo-3,4-dihydrobenzo [g]pteridin-10 (2H)-yl) ethoxy) methyl) piperidine-1-carboxylate

### Batch ID: STL-7-A-574-JG-2009-154-Boc

To a solution of tert-butyl 4-((2-((2-amino-4-chloro-5-ethylphenyl) amino) ethoxy) methyl) piperidine-1-carboxylate (JG-2009-A3) (0.24 g, 0.58 mmol, 1.0 eq) in AcOH (5.0 mL), alloxan monohydrate (CAS: 2244-11-3) (0.093 g, 0.58 mmol, 1.0 eq) and boric anhydride (0.081 g, 1.10 mmol, 2.0 eq) were added and reaction mixture was stirred at 50° C. for 5 min. After completion of reaction as indicated by TLC the reaction mixture was quenched with ice cold water (30 mL) slowly and stirred for 20 min, the solid was precipitate out. The reaction mass was filtered over micro buckner funnel, solid was washed by ice cold water (5 ml×2). Then solid was dried under reduced pressure to afford reddish brown solid. The crude was triturated with acetonitrile (10 ml×2) to obtain title compound as yellow solid (0.01 g, 3.0%). LCMS: m/z 518.1 & 520.1 (M & M+2).

### Step-5: Synthesis of 2,2,2-trifluoroacetaldehyde compound with 7-chloro-8-ethyl-10-(2-(piperidin-4-ylmethoxy) ethyl) benzo [g]pteridine-2,4(3H, 10H)-dione (JG-2009)

### Batch ID: STL-7-A-574-JG-2009-174

To a solution of tert-butyl 4-((2-(7-chloro-8-ethyl-2,4-dioxo-3,4-dihydrobenzo [g]pteridin-10 (2H)-yl) ethoxy) methyl) piperidine-1-carboxylate (JG-2009-Boc) (0.02 g, 0.038 mmol, 1 eq) in DCM (2 mL), Trifluoroacetic acid (0.011 mL, 0.15 mmol, 4.0 eq) was added at 0° C. and the reaction mixture was allowed to stir for 2 hr at room temperature. After completion of reaction as indicated by TLC, the reaction mixture was concentrated under vacuum and dried under reduced pressure. The crude was triturated with diethyl ether (10 ml×2) to obtained title compound as yellow solid (0.013 g, 81.25%). LCMS: m/z 418.1 & 420.0 (M & M+2); 1H NMR: (400 Mz, DMSO) δ 11.49 (s, 1H), 8.40 (bs, 2H), 8.21 (s, 1H), 8.00 (s, 1H), 4.80 (t, 2H), 3.81 (t, 2H), 3.26 (d, J=6.4 Hz, 2H), 3.10-3.06 (m, 2H), 2.93 (q, J=7.2 Hz, 2H), 2.85-2.60 (m, 21H), 1.75-1.55 (m, 31H), 1.28 (t, J=7.6 Hz, 3H), 1.20-1.10 (m, 2H).

The following compounds were made according to the procedure described in above examples using JG-2001-X and various amine side chains.

| Target ID | Structure | Analytical data |
|---|---|---|
| JG-2006 | 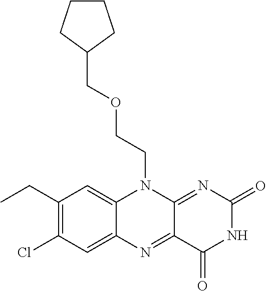 [SMILES: CCc1cc2c(cc1Cl)nc1c(=O)[nH]c(=O)nc-1n2CCOCC1CCCC1] | LCMS m/z 403.1 & 405.1 (M & M + 2); 1H NMR (400 MHz, DMSO) δ 11.44 (s, 1H), 8.19 (s, 1H), 8.03 (s, 1H), 4.80 (t, 2H), 3.79 (t, J = 4.8 Hz, 2H), 3.23 (d, J = 6.8 Hz, 2H), 2.91 (q, J = 7.2 Hz, 2H), 1.95-1.88 (m, 1H), 1.49-1.38 (m, 6H), 1.27 (t, J = 7.6 Hz, 3H), 1.04-0.95 (m, 2H). |
| JG-2015 | 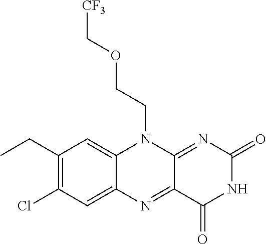 [SMILES: CCc1cc2c(cc1Cl)nc1c(=O)[nH]c(=O)nc-1n2CCOCC(F)(F)F] | LCMS m/z 403.0 & 405.1 (M & M + 2); 1H NMR (400 MHz, DMSO) δ 11.46 (s, 1H), 8.20 (s, 1H), 8.00 (s, 1H), 4.85 (s, 2H), 4.09-4.00 (m, 4H), 2.91 (q, J = 7.2 Hz, 2H), 1.27 (t, J = 7.6 Hz, 3H). |
| JG-2008 | 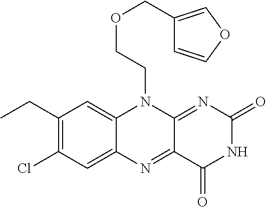 [SMILES: CCc1cc2c(cc1Cl)nc1c(=O)[nH]c(=O)nc-1n2CCOCc1ccoc1] | LCMS m/z 401.0 & 403.0 (M & M + 2); 1H NMR (400 MHz, DMSO) δ 11.44 (s, 1H), 8.19 (s, 1H), 7.99 (s, 1H), 7.55-7.53 (m, 2H), 6.24 (s, 1H), 4.80 (t, 2H), 4.33 (s, 2H), 3.83 (t, 2H), 2.89 (q, J = 7.2 Hz, 2H), 1.20 (t, J = 7.6 Hz, 3H). |
| JG-2092 | 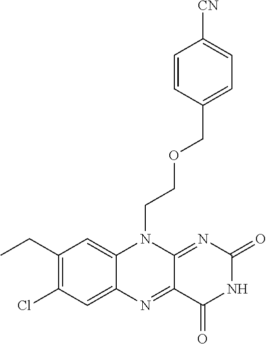 [SMILES: CCc1cc2c(cc1Cl)nc1c(=O)[nH]c(=O)nc-1n2CCOCc1ccc(C#N)cc1] | LCMS m/z 436.0 & 438.0 (M & M + 2); 1H NMR (400 MHz, DMSO) δ 11.45 (s, 1H), 8.19 (s, 1H), 8.02 (s, 1H), 7.71 (d, J = 8.0 Hz, 2H), 7.31 (d, J = 8.0 Hz, 2H), 4.87 (bs, 2H), 4.56 (s, 2H), 3.93 (bs, 2H), 2.87 (q, J = 7.2 Hz, 2H), 1.15 (t, J = 7.2 Hz, 3H). |

### Synthesis of phenolic ether derivatives (JG-2082, JG-2067, JG-2074, JG-2076, JG-2077, JG-2078, JG-2086, JG-2087, JG-2088, JG-2034, JG-2079, JG-2080, JG-2081, JG-2083, JG-2089, JG-2090)

### Synthesis of 7-chloro-8-ethyl-10-(2-(p-tolyloxy)ethyl)benzo[g]pteridine-2,4(3H, 10H)-dione (JG-2082)

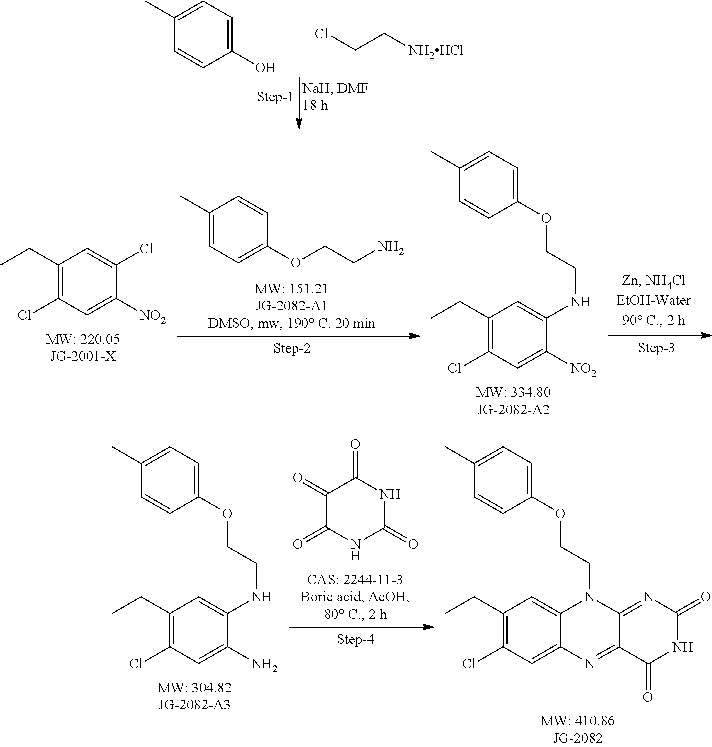

### Step-1 Synthesis of 2-(p-tolyloxy)ethan-1-amine

### Batch ID: STL7-A574-JG-2082-A1-138

To a solution of p-cresol (0.20 g, 1.85 mmol, 1 eq) and 2-chloroethylamine hydrochloride (0.21 g, 1.85 mmol, 1.0 eq) in DMF (35 mL), sodium hydride (0.46 g, 9.25 mmol, 5 eq) was added and the reaction mixture was stirred at room temperature for 18 h at room temperature. The reaction mixture was quenched with ice water (25 mL) and extracted with ethyl acetate (3×50 mL). The combined organic phase was dried over anhydrous sodium sulphate and concentrated under vacuum. The crude product was purified by column chromatography to obtain title compound as white gummy solid. (0.12 g, 42.8%). LCMS m/z 152.02 (M+1).

### Step-2 Synthesis of 4-chloro-5-ethyl-2-nitro-N-(2-(p-tolyloxy)ethyl)aniline

### Batch ID: STL7-A574-JG-2082-A2-141

A solution of 2-(p-tolyloxy)ethan-1-amine (JG-2082-A1) (0.120 g, 0.794 mmol, 1 eq) and 1,4-dichloro-2-ethyl-5-nitrobenzene (JG-2001-X) (0.21 g, 0.794 mmol, 1.0 eq), in DMSO (2 mL) was heated at 190° C. under microwave irradiation for 20 min. The reaction mixture was poured into water (25 mL) and extracted with ethyl acetate (2×25 mL). The organic phase was dried over anhydrous sodium sulphate and concentrated under vacuum. The crude was purified by column chromatography (20% ethyl acetate/hexane) to obtain title compound as yellow solid. (0.100 g, 37.70%). LCMS m/z 335.13 (M+1).

### Step-3 Synthesis of 4-chloro-5-ethyl-N1-(2-(p-tolyloxy)ethyl)benzene-1,2-diamine

### Batch ID: STL7-A574-JG-2082-A3-142

To a solution of 4-chloro-5-ethyl-2-nitro-N-(2-(p-tolyloxy)ethyl)aniline (JG-2082-A2) (0.10 g, 0.299 mmol, 1 eq) in ethanol (10 mL) and water (2 mL), Zinc dust (0.156 g, 2.39 mmol, 8 eq) and ammonium chloride (0.128 g, 2.39 mmol, 8 eq) were added and the reaction mixture was stirred at 90° C. 2 h. After completion of reaction as indicated by TLC, the reaction mixture was filtered through celite and washed with ethyl acetate (50 ml). The filtrate was washed with water (50 ml) and the organic phase was dried over anhydrous sodium sulphate. The combined organic layer was concentrated under vacuum to afford crude as yellow liquid. (0.080 g, 88.01%). LCMS m/z 305.1 & 307.1 (M & M+2).

### Step-4 Synthesis of 7-chloro-8-ethyl-10-(2-(p-tolyloxy)ethyl)benzo[g]pteridine-2,4 (3H, 10H)-dione

### Batch ID: STL7-A574-JG-2082-A4-143

To a solution of 4-chloro-5-ethyl-N₁-(2-(p-tolyloxy)ethyl)benzene-1,2-diamine (JG-2082-A3) (0.080 g, 0.29 mmol, 1.0 eq) in AcOH (30 mL) (CAS: 2244-11-3) (0.042 g, 0.296 mmol, 1.0 eq) and Boric anhydride (0.041 g, 0.592 mmol, 2.0 eq) were added and the reaction mixture was stirred at 80° C. for 2 h. The reaction mixture was quenched with ice cooled water (35 mL) and extracted with ethyl acetate (2×50 mL). The combined organic phase was dried over anhydrous sodium sulphate and concentrated under reduce pressure. The obtained crude product was further purified by column chromatography (100% ethyl acetate) to afford yellow solid (0.010 g, 8.24%) LCMS m/z 410.9 & 413.2 (M & M+2); 1H NMR (400 MHz, DMSO): 5: 11.45 (s, 1H), 8.21 (s, 1H), 8.10 (s, 1H), 7.05 (d, J=8.4 Hz, 2H), 6.75 (d, J=8.0 Hz, 2H), 4.99 (t, 2H), 4.37 (t, 2H), 2.92 (q, 2H), 2.19 (s, 31H), 1.28 (t, J=7.6 Hz, 3H).

The following compounds were made according to the procedure described for JG-2082 using JG-2001-X and the respective amine.

| Target ID | Structure | Analytical data |
|---|---|---|
| JG-2067 | 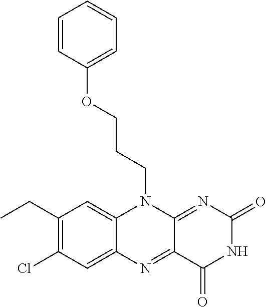 [SMILES: CCc1cc2c(cc1Cl)nc1c(=O)[nH]c(=O)nc-1n2CCCOc1ccccc1] | LCMS m/z 411.0 & 413.0 (M & M + 2); 1H NMR (400 MHz, DMSO): δ: 11.42 (s, 1H), 8.22 (s, 1H), 7.90 (s, 1H), 7.92-7.25 (m, 2H), 6.94-6.85 (m, 3H), 4.76 (bs, 2H), 4.11 (t, 2H), 2.82 (q, J = 7.6 Hz, 2H), 2.24 (bs, 2H), 1.16 (t, J = 7.2 Hz, 3H). |
| JG-2074 | 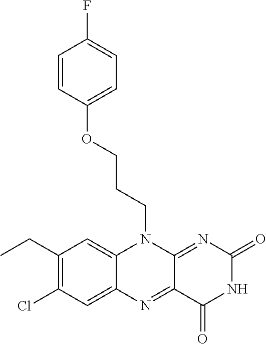 [SMILES: CCc1cc2c(cc1Cl)nc1c(=O)[nH]c(=O)nc-1n2CCCOc1ccc(F)cc1] | LCMS m/z 429.0 & 431.0 (M & M + 2); 1H NMR (400 MHz, DMSO, ppm) δ 11.43 (s, 1H), 8.20 (s, 1H), 7.86 (s, 1H), 7.08 (t, J = 8.8 Hz, 2H), 6.84 (m, J = 4 Hz, 2H), 4.75 (t, 2H), 4.07 (t, J = 5.6 Hz, 2H), 2.81 (q, J = 7.6 Hz, 2H), 2.21 (m, J = 6.4 Hz, 2H), 1.14 (t, J = 7.2 Hz, 3H) |
| JG-2076 | 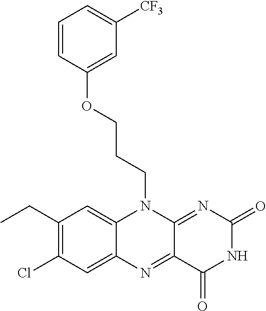 [SMILES: CCc1cc2c(cc1Cl)nc1c(=O)[nH]c(=O)nc-1n2CCCOc1cccc(C(F)(F)F)c1] | LCMS m/z 479.0 & 481.0 (M & M + 2); 1H NMR (400 MHz, DMSO): δ: 11.43 (s, 1H), 8.21 (s, 1H), 7.91 (s, 1H), 7.52-7.48 (m, 1H), 7.28-7.11 (m, 3H), 4.77 (t, 2H), 4.20 (bs, 2H), 2.80 (q, J = 7.6 Hz, 2H), 2.30-2.20 (m, 2H), 1.12 (t, J = 7.2 Hz, 3H). |
| JG-2077 | 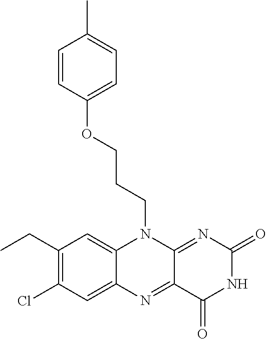 [SMILES: CCc1cc2c(cc1Cl)nc1c(=O)[nH]c(=O)nc-1n2CCCOc1ccc(C)cc1] | LCMS m/z 425.0 & 427.0 (M & M + 2); 1H NMR (400 MHz, DMSO): δ: 11.42 (s, 1H), 8.21 (s, 1H), 7.88 (s, 1H), 7.06 (d, J = 8.0 Hz, 2H), 6.76 (d, J = 8.4 Hz, 2H), 4.74 (t, 2H), 4.08 (t, J = 5.6 Hz, 2H), 2.82 (q, J = 7.6 Hz, 2H), 2.21 (s, 3H), 2.20-2.15 (m, 2H), 1.17 (t, J = 7.2 Hz, 3H). |
| JG-2078 | 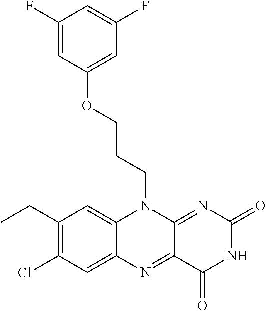 [SMILES: CCc1cc2c(cc1Cl)nc1c(=O)[nH]c(=O)nc-1n2CCCOc1cc(F)cc(F)c1] | LCMS m/z 447.0 & 449.0 (M & M + 2); 1H NMR (400 MHz, DMSO): δ: 11.44 (s, 1H), 8.23 (s, 1H), 7.89 (s, 1H), 6.80-6.75 (m, 1H), 6.64-6.61 (m, 2H), 4.74 (t, 2H), 4.15 (t, J = 5.6 Hz, 2H), 2.85 (q, J = 7.6 Hz, 2H), 2.24-2.21 (m, 2H), 1.18 (t, J = 7.6 Hz, 3H). |
| JG-2086 | 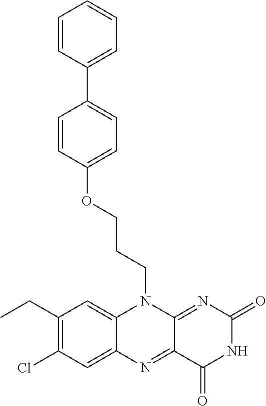 [SMILES: CCc1cc2c(cc1Cl)nc1c(=O)[nH]c(=O)nc-1n2CCCOc1ccc(-c2ccccc2)cc1] | LCMS m/z 487.0 & 489.0 (M & M + 2); 1H NMR (400 MHz, DMSO): δ: 11.44 (s, 1H), 8.21 (s, 1H), 7.90 (s, 1H), 7.61-7.56 (m, 4H), 7.41 (dd, J = 7.6 Hz, 2H), 7.30 (t, J = 7.2 Hz, 1H), 6.95 (d, J = 8.4 Hz, 2H), 4.75 (t, 2H), 4.17 (t, J = 5.6 Hz, 2H), 2.82 (q, J = 7.2 Hz, 2H), 2.28-2.25 (m, 2H), 1.16 (t, J = 7.2 Hz, 3H). |
| JG-2087 | 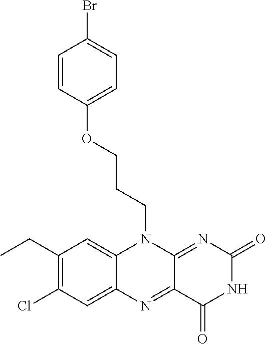 [SMILES: CCc1cc2c(cc1Cl)nc1c(=O)[nH]c(=O)nc-1n2CCCOc1ccc(Br)cc1] | LCMS m/z 490.9 & 492.9 (M & M + 2); 1H NMR (400 MHz, DMSO): δ: 11.43 (s, 1H), 8.21 (s, 1H), 7.88 (s, 1H), 7.43 (d, J = 8.4 Hz, 2H), 6.84 (d, J = 8.8 Hz, 2H), 4.73 (bs, 2H), 4.11 (t, 2H), 2.82 (q, J = 7.2 Hz, 2H), 2.30-2.25 (m, 2H), 1.17 (t, J = 7.2 Hz, 3H). |
| JG-2088 | 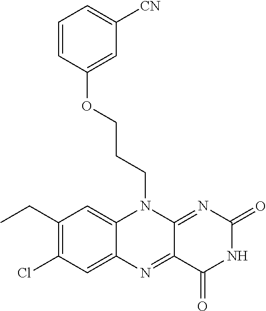 [SMILES: CCc1cc2c(cc1Cl)nc1c(=O)[nH]c(=O)nc-1n2CCCOc1cccc(C#N)c1] | LCMS m/z 436.1 & 438.1 (M & M + 2); 1H NMR (400 MHz, DMSO): δ: 11.44 (s, 1H), 8.22 (s, 1H), 7.90 (s, 1H), 7.49-7.38 (m, 2H), 7.33 (s, 1H), 7.20 (dd, J = 8.0 & 1.6 Hz, 1H), 4.75 (t, 2H), 4.19 (t, J = 5.6 Hz, 2H), 2.83 (q, J = 7.6 Hz, 2H), 2.26-2.23 (m, 2H), 1.16 (t, J = 7.2 Hz, 3H). |
| JG-2034 | 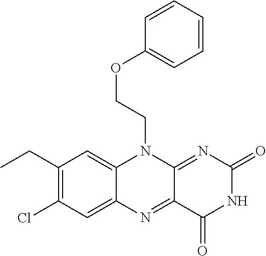 [SMILES: CCc1cc2c(cc1Cl)nc1c(=O)[nH]c(=O)nc-1n2CCOc1ccccc1] | LCMS m/z 397.50 & 399.62 (M & M + 2); 1H NMR (400 MHz, DMSO): δ: 11.46 (s, 1H), 8.22 (s, 1H), 8.13 (s, 1H), 7.26 (t, J = 8 Hz, 2H), 6.94-6.85 (m, 3H), 5.02 (t, 2H), 4.43 (bs, 2H), 2.93 (q, J = 7.2 Hz, 2H), 1.29 (t, J = 7.6 Hz, 3H). |
| JG-2079 | 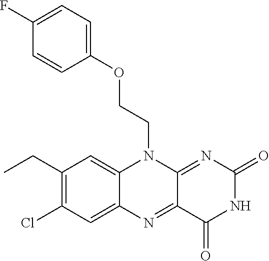 [SMILES: CCc1cc2c(cc1Cl)nc1c(=O)[nH]c(=O)nc-1n2CCOc1ccc(F)cc1] | LCMS m/z 415.0 & 417.0 (M & M + 2); 1H NMR (400 MHz, DMSO): δ: 11.45 (s, 1H), 8.21 (s, 1H), 8.11 (s, 1H), 7.10-7.06 (m, 2H), 6.88-6.84 (m, 2H), 5.00 (t, 2H), 4.39 (t, 2H), 2.92 (q, J = 7.2 Hz, 2H), 1.26 (t, J = 7.6 Hz, 3H). |
| JG-2080 | 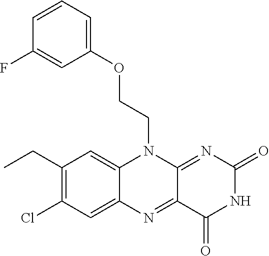 [SMILES: CCc1cc2c(cc1Cl)nc1c(=O)[nH]c(=O)nc-1n2CCOc1cccc(F)c1] | LCMS m/z 415.0 & 417.1 (M & M + 2); 1H NMR (400 MHz, DMSO): δ: 11.46 (s, 1H), 8.21 (s, 1H), 8.09 (s, 1H), 7.28-7.25 (m, 1H), 6.76-6.67 (m, 3H), 5.01 (t, 2H), 4.44 (t, 2H), 2.92 (q, J = 7.2 Hz, 2H), 1.26 (t, J = 7.6 Hz, 3H). |
| JG-2081 | 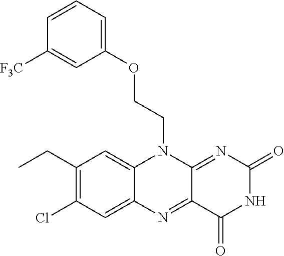 [SMILES: CCc1cc2c(cc1Cl)nc1c(=O)[nH]c(=O)nc-1n2CCOc1cccc(C(F)(F)F)c1] | LCMS m/z 465.0 & 467.0 (M & M + 2); 1H NMR (400 MHz, DMSO): δ: 11.46 (s, 1H), 8.21 (s, 1H), 8.13 (s, 1H), 7.48 (t, J = 7.6 Hz, 1H), 7.27 (d, J = 7.2 Hz, 1H), 7.17 (t, J = 8 Hz, 1H), 7.09 (s, 1H), 5.04 (t, 2H), 4.51 (t, 2H), 2.91 (q, J = 7.2 Hz, 2H), 1.28 (t, 3H). |
| JG-2083 | 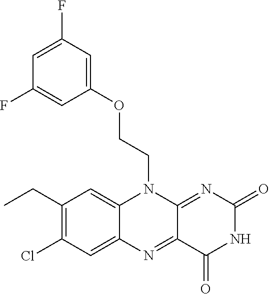 [SMILES: CCc1cc2c(cc1Cl)nc1c(=O)[nH]c(=O)nc-1n2CCOc1cc(F)cc(F)c1] | LCMS m/z 433.0 & 435.0 (M & M + 2); 1H NMR (400 MHz, DMSO): δ: 11.46 (s, 1H), 8.21 (s, 1H), 8.05 (s, 1H), 6.79-6.77 (m, 1H), 6.65-6.63 (m, 2H), 5.01 (t, 2H), 4.46 (t, 2H), 2.92 (q, J = 7.2 Hz, 2H), 1.27 (t, J = 7.6 Hz, 3H). |
| JG-2089 | 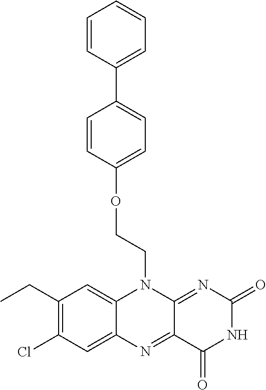 [SMILES: CCc1cc2c(cc1Cl)nc1c(=O)[nH]c(=O)nc-1n2CCOc1ccc(-c2ccccc2)cc1] | LCMS m/z 473.2 & 475.1 (M & M + 2); 1H NMR (400 MHz, DMSO): δ: 11.45 (s, 1H), 8.21 (s, 1H), 8.13 (s, 1H), 7.57-7.55 (m, 4H), 7.42-7.27 (m, 3H), 6.95 (d, J=, 2H), 5.04 (t, 2H), 4.48 (t, 2H), 2.93 (q, J = 7.2 Hz, 2H), 1.29 (t, J = 7.2 Hz, 3H). |
| JG-2090 | 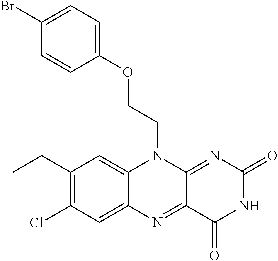 [SMILES: CCc1cc2c(cc1Cl)nc1c(=O)[nH]c(=O)nc-1n2CCOc1ccc(Br)cc1] | LCMS m/z 474.8 & 476.9 (M & M + 2); 1H NMR (400 MHz, DMSO): δ: 11.46 (s, 1H), 8.21 (s, 1H), 8.11 (s, 1H), 7.42 (d, J = 8.8 Hz, 2H), 6.83 (d, J = 8.8 Hz, 2H), 5.00 (t, 2H), 4.41 (t, 2H), 2.93 (q, 2H), 1.28 (t, J = 7.6 Hz, 3H). |

### Synthesis of amine sidechain derivatives (JG-2025, JG-2052, JG-2051, JG-2053)

### Synthesis of 10-(2-aminoethyl)-7-chloro-8-ethylbenzo[g]pteridine-2,4(3H, 10H)-dione 2,2,2-trifluoroacetate (JG-2025)

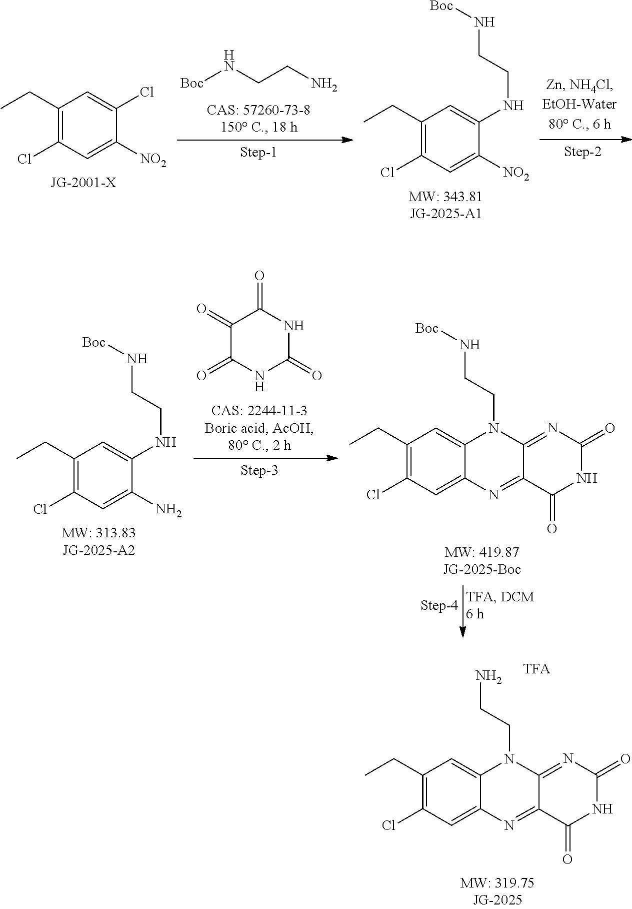

### Step-1 Synthesis of tert-butyl (2-((4-chloro-5-ethyl-2-nitrophenyl)amino)ethyl)carbamate

### Batch ID: STL7-A574-JG-2025-A1-060

1,4-dichloro-2-ethyl-5-nitrobenzene (JG-2001-X) (3.0 g, 13.63 mmol, 1 eq) and tert-butyl (2-aminoethyl)carbamate (CAS: 57260-73-8) (2.18 g, 13.63 mmol, 1.0 eq) were stirred neat at 150° C. temperature. After 18 h of stirring TLC analysis confirms consumption of SM, the reaction mixture was slowly poured into water (100 mL) and extracted with ethyl acetate (2×75 mL). The organic phase was dried over anhydrous sodium sulphate and concentrated under vacuum. The crude material was purified by column chromatography (30% ethyl acetate/hexane) as yellow solid (3.01 g, 64.3%). LCMS: MS: m/z 344.6 & 346.6 (M+1).

### Step-2 Synthesis of tert-butyl (2-((2-amino-4-chloro-5-ethylphenyl)amino)ethyl) carbamate

### Batch ID: STL7-A574-JG-2025-A2-066

To a solution of tert-butyl (2-((4-chloro-5-ethyl-2-nitrophenyl)amino)ethyl) carbamate (JG-2025-A1) (3.0 g, 8.72 mmol) in ethanol:water (8:2V, 40 mL), Zn Dust (4.56 g, 69.8 mmol, 8.0 eq) and NH4Cl (3.73 g, 69.8 mmol, 8.0 eq) were added and the reaction mixture was stirred at 80° C. Reaction progress was further monitored using TLC (mobile phase: 20% ethyl acetate in hexane), after 6 h of stirring TLC analysis confirms consumption of SM. The reaction mixture was filtered through celite and filtrate was poured into water (75 mL) and extracted with ethyl acetate (2×100 mL). The combined organic phase was dried over anhydrous sodium sulphate and concentrated under reduce pressure. The obtained crude product material was directly used in next step (2.8 g, Quantitative). LCMS: m/z 314.6 & 316.6 (M & M+2).

### Step-3 Synthesis of tert-butyl (2-(7-chloro-8-ethyl-2,4-dioxo-3,4-dihydrobenzo[g]pteridin-10(2H)-yl)ethyl)carbamate

### Batch ID: STL7-A574-JG-2025-A4-067

To a solution of tert-butyl (2-((2-amino-4-chloro-5-ethylphenyl)amino)ethyl) carbamate (JG-2025-A2) (2.75 g, 8.70 mmol, 1.0 eq) in AcOH (30 mL), alloxan monohydrate (CAS: 2244-11-3) (1.54 g, 8.70 mmol, 1.0 eq) and boric anhydride (1.36 g, 17.53 mmol, 2.0 eq) were added. And the reaction mixture was stirred at 80° C. temperature. Reaction progress was further monitored using TLC (mobile phase: 100% ethyl acetate in hexane), after 2 h of stirring TLC analysis confirms consumption of SM. The reaction mixture was slowly poured in to ice cooled water (100 mL) extracted with ethyl acetate (2×100 mL). The organic phase was dried over anhydrous sodium sulphate and concentrated under reduced pressure. The obtained crude product was further purified by column chromatography (100% ethyl acetate) to obtain yellow solid (2.15 g, 58.9%) LCMS: m/z 420.80 & 422.8 (M& M+2).

### Step-4 Synthesis of 10-(2-aminoethyl)-7-chloro-8-ethylbenzo[g]pteridine-2,4(3H, 10H)-dione 2,2,2-trifluoroacetate

### Batch ID: STL7-A574-JG-2025-A4-069

To a solution of tert-butyl (2-(7-chloro-8-ethyl-2,4-dioxo-3,4-dihydrobenzo[g]pteridin-10(2H)-yl)ethyl)carbamate (JG-2025-Boc) (2.0 g, 4.77 mmol, 1.0 eq) in DCM (30 mL), TFA (10.86 g, 95.2 mmol, 20.0 eq) was added and the reaction mixture was stirred at room temperature for 6 h. Reaction progress was further monitored using TLC (100% ethyl acetate/hexane). The reaction mixture was concentrated under vacuum, triturated with MTBE and diethyl ether and again dried under vacuum to afford product as yellow solid (1.85 g, 90.2%). LCMS: m/z 320.51 (M+1); 1H NMR (400 MHz, DMSO): 5: 11.58 (s, 1H), 8.29 (s, 1H), 7.95 (bs, 4H), 4.87 (t, 2H), 3.23 (bs, 2H), 2.96 (q, 2H), 1.31 (t, 3H).

### Synthesis of N-(2-(7-chloro-8-ethyl-2,4-dioxo-3,4-dihydrobenzo[g]pteridin-10(2H)-yl)ethyl)acetamide (JG-2052)

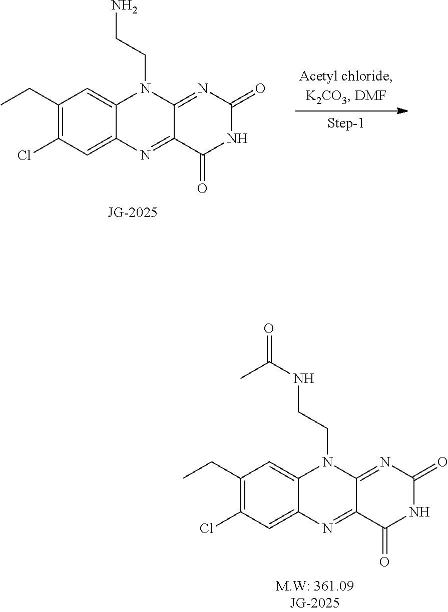 [SMILES: CCc1cc2c(cc1Cl)nc1c(=O)[nH]c(=O)nc-1n2CCN ; CCc1cc2c(cc1Cl)nc1c(=O)[nH]c(=O)nc-1n2CCNC(C)=O]

### Batch ID: STL7-A574-JG-2025-A5-036

To a solution of 10-(2-aminoethyl)-7-chloro-8-ethylbenzo[g]pteridine-2,4(3H, 10H)-dione (JG-2025) (0.050 g, 0.156 mmol, 1 eq) in DMF (2 mL), potassium carbonate (0.064 g, 0.468 mmol, 3 eq) and acetyl chloride (0.014 mg, 0.188 mmol, 1.2 eq) were added and the reaction mixture was stirred at room temperature for 18 h. The reaction mixture was poured into water (25 mL) and extracted with ethyl acetate (75 mL). The organic phase was dried over anhydrous sodium sulphate and concentrated. The crude material was purified by column chromatography (5% methanol/dichloromethane) to obtain title compound as yellow solid (2 mg, 3.55%). LCMS: m/z 362.1 & 364.3 (M& M+2); 1H NMR (400 MHz, DMSO): 5: 11.49 (s, 1H), 8.24 (s, 1H), 8.16 (t, 1H), 8.06 (s, 1H), 4.62 (t, 2H), 3.43 (t, 2H), 2.94 (q, 2H), 1.71 (s, 3H), 1.32 (t, 3H).

### Synthesis of N-(2-(7-chloro-8-ethyl-2,4-dioxo-3,4-dihydrobenzo[g]pteridin-10(2H)-yl)ethyl) benzamide (JG-2051)

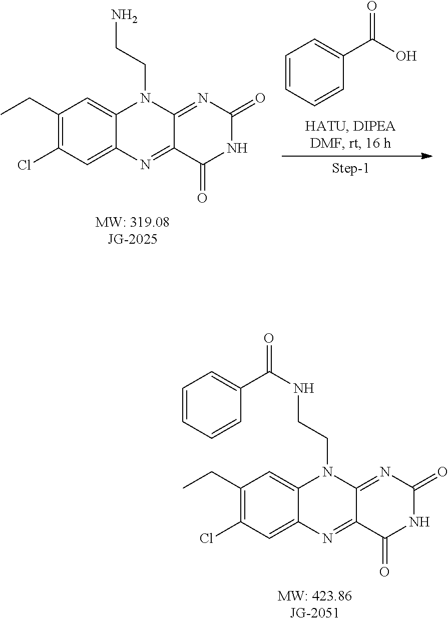

### Batch ID: STL7-A574-JG-2025-A5-040

To a solution of 10-(2-aminoethyl)-7-chloro-8-ethylbenzo[g]pteridine-2,4(3H, 10H)-dione (JG-2025) (0.076 g, 0.63 mmol, 1 eq) in DMF (2 mL), DIPEA (0.30 mL, 1.88 mmol, 3eq), HATU (0.36 g, 0.94 mmol, 1.5eq) and benzoic acid (0.20 g, 0.63 mmol, 1eq) were added. The reaction mixture was stirred at room temperature for 18 h. The reaction mixture was poured into water (25 mL) and extracted with ethyl acetate (2×25 mL). The combined organic phase was dried over anhydrous sodium sulphate and concentrated under vacuum. The crude material was purified by column chromatography (100% ethyl acetate) to obtain title compound as yellow solid (0.030 g, 11.2%). LCMS m/z 424.6 & 426.6 (M & M+2); 1H NMR (400 MHz, DMSO): 5: 11.53 (s, 1H), 8.65 (t, 1H), 8.22 (s, 1H), 8.01 (s, 1H), 7.61 (d, J=7.2 Hz, 2H), 7.51-7.37 (m, 3H), 4.79 (t, 2H), 3.79-3.72 (m, 2H), 2.76 (q, 2H), 1.14 (t, 3H).

The following compound was made according to the procedure described for JG-2051 using JG-2025 and isobutyric acid.

| Target ID | Structure | Analytical data |
|---|---|---|
| JG-2053 | 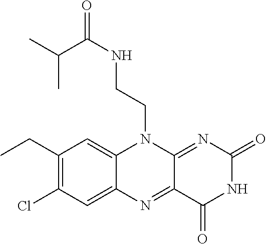 [SMILES: CCc1cc2c(cc1Cl)nc1c(=O)[nH]c(=O)nc-1n2CCNC(=O)C(C)C] | LCMS m/z 390.7 & 392.7 (M & M + 2); 1H NMR (400 MHz, DMSO): δ: 11.50 (s, 1H), 8.24 (s, 1H), 8.03 (s, 1H), 7.97 (t, 1H), 4.67 (t, 2H), 3.55 (m, 2H), 2.94 (q, J = 7.6 Hz, 2H), 2.14 (hept, 1H), 1.34 (t, J = 7.2 Hz, 3H), 0.81 (d, J = 6.8 Hz, 6H). |

### Synthesis of sulfonamide sidechain derivatives (JG-2057, JG-2061, JG-2060, JG-2062, JG-2063, JG-2064, JG-2065)

### Synthesis of N-(2-(7-chloro-8-ethyl-2,4-dioxo-3,4-dihydrobenzo[g]pteridin-10(2H)-yl)ethyl) benzene sulfonamide (JG-2057)

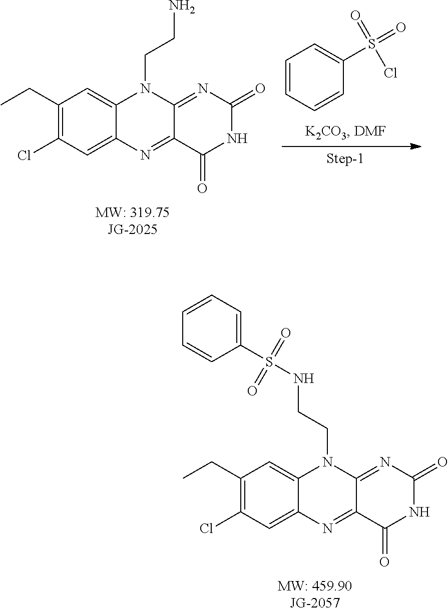

### Batch ID: STL7-A574-JG-2025-A5-055

To a solution of 10-(2-aminoethyl)-7-chloro-8-ethylbenzo[g]pteridine-2,4(3H, 10H)-dione (JG-2025) (0.100 g, 0.313 mmol, 1 eq) in DMF (2 mL), potassium carbonate (0.13 g, 0.939 mmol, 3 eq) and benzenesulfonyl chloride (0.083 g, 0.188 mmol, 1.2 eq) were added and stirred for 18 h at room temperature. The reaction mixture was poured into water (50 mL) and extracted with ethyl acetate (2×25 mL). The combined organic phase was dried over anhydrous sodium sulphate and concentrated under vacuum. The crude material was purified by column chromatography (100% ethyl acetate) as yellow solid (0.012 g, 8.35%). LCMS: m/z 460.78 (M+1); 1H NMR (400 MHz, DMSO): δ: 11.50 (s, 1H), 8.22 (s, 1H), 7.97 (t, 1H), 7.91 (s, 1H), 7.72 (d, J=7.6 Hz, 2H), 7.62-7.51 (m, 3H), 4.66 (t, 2H), 3.23-21 (m, 2H), 2.93 (q, 2H), 1.32 (t, 3H).

The following compounds were made according to the procedure described for JG-2057 using JG-2025 and respective sulfonyl chloride.

| Target ID | Structrure | Analytical data |
|---|---|---|
| JG-2062 | 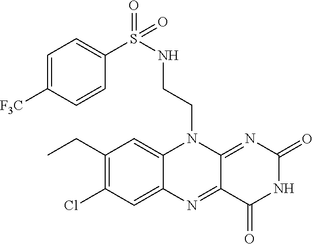 [SMILES: CCc1cc2c(cc1Cl)nc1c(=O)[nH]c(=O)nc-1n2CCNS(=O)(=O)c1ccc(C(F)(F)F)cc1] | LCMS: m/z 428.2 & 430.1 (M & M + 2); 1H NMR (400 MHz, DMSO): δ: 11.51 (s, 1H), 8.20 (bs, 1H), 8.18 (s, 1H), 7.95-7.89 (m, 5H), 4.66 (t, 2H), 3.29 (t, 2H), 2.90 (q, J = 7.2 Hz, 2H), 1.30 (t, J = 7.6 Hz, 3H). |
| JG-2063 | 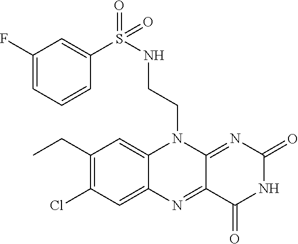 [SMILES: CCc1cc2c(cc1Cl)nc1c(=O)[nH]c(=O)nc-1n2CCNS(=O)(=O)c1cccc(F)c1] | LCMS: m/z 478.1 & 480.1 (M & M + 2); 1H NMR (400 MHz, DMSO): δ: 11.50 (s, 1H), 8.20 (s, 1H), 8.10 (bs, 1H), 7.90 (s, 1H), 7.58-7.44 (m, 4H), 4.66 (t, 2H), 3.27 (t, 2H), 2.92 (q, J = 7.6 Hz, 2H), 1.31 (t, J = 7.6 Hz, 3H). |
| JG-2064 | 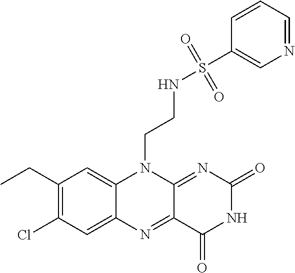 [SMILES: CCc1cc2c(cc1Cl)nc1c(=O)[nH]c(=O)nc-1n2CCNS(=O)(=O)c1cccnc1] | LCMS: m/z 461.1 & 463.2 (M & M + 2); 1H NMR (400 MHz, DMSO): δ: 11.50 (s, 1H), 8.86 (s, 1H), 8.77 (d, J = 3.6 Hz, 1H), 8.30 (bs, 1H), 8.21 (s, 1H), 8.13-8.11 (m, 1H), 7.96 (s, 1H), 7.58- 7.54 (m, 1H), 4.66 (t, 2H), 3.52-3.40 (m, 2H), 2.92 (q, J = 7.2 Hz, 2H), 1.31 (t, J = 7.2 Hz, 3H). |

### Synthesis of N-(2-(7-chloro-8-ethyl-2,4-dioxo-3,4-dihydrobenzo[g]pteridin-10(2H)-yl)ethyl)-2-methyl propane-i-sulfonamide (JG-2061)

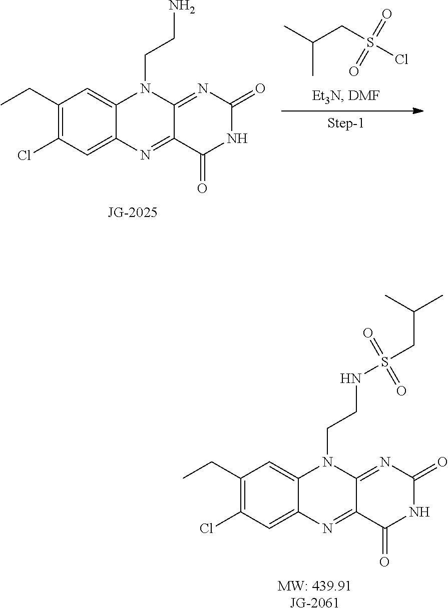

### Batch ID: STL7-A574-JG-2025-A5-087

To a solution of 10-(2-aminoethyl)-7-chloro-8-ethylbenzo[g]pteridine-2,4(3H, 10H)-dione (JG-2025) (0.050 g, 0.156 mmol, 1 eq) in DMF (3 mL), triethylamine (0.06 mL, 0.468 mmol, 3eq) and isobutylsulfonyl chloride (0.025 g, 0.156 mmol, 1eq) were added and the reaction mixture was stirred at room temperature for 18 h. The reaction mixture was poured into water (50 mL) and extracted with ethyl acetate (2×25 mL). The combined organic phase was dried over anhydrous sodium sulphate and concentrated under vacuum. The crude material was purified by column chromatography (100% ethyl acetate) to obtain title compound as yellow solid (0.012 g, 8.35%). LCMS: m/z 440.3 & 442.3 (M & M+2); 1H NMR (400 MHz, DMSO): δ: 11.50 (s, 1H), 8.24 (s, 1H), 7.97 (s, 1H), 4.68 (t, 2H), 3.39 (t, 2H), 2.95-2.90 (m, 4H), 2.00 (hept, 1H), 1.32 (t, J=7.6 Hz, 3H), 0.97 (d, J=6.4 Hz, 6H).

The following compounds were made according to the procedure described for JG-2061 using JG-2025 and respective sulfonyl chloride.

| Target ID | Structure | Analytical data |
|---|---|---|
| JG-2060 | 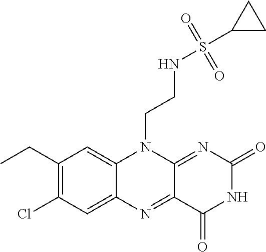 [SMILES: CCc1cc2c(cc1Cl)nc1c(=O)[nH]c(=O)nc-1n2CCNS(=O)(=O)C1CC1] | LCMS: m/z 424.0 & 426.0 (M & M + 2); 1H NMR (400 MHz, DMSO): δ: 11.49 (s, 1H), 8.23 (s, 1H), 7.95 (s, 1H), 7.33 (t, 1H), 4.70 (t, 2H), 3.50-3.40 (m, 2H), 2.92 (q, 2H), 2.60-2.40 (m, 1H), 1.30 (t, 3H), 0.91-0.86 (m, 4H). |
| JG-2065 | 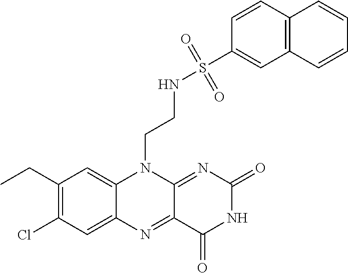 [SMILES: CCc1cc2c(cc1Cl)nc1c(=O)[nH]c(=O)nc-1n2CCNS(=O)(=O)c1ccc2ccccc2c1] | LCMS: m/z 510.2 & 512.2 (M & M + 2); 1H NMR (400 MHz, DMSO): δ: 11.47 (s, 1H), 8.34 (s, 1H), 8.06-7.97 (m, 5H), 7.97 (s, 1H), 7.68- 7.63 (m, 3H), 4.64 (t, 2H), 3.39-3.30 (m, 2H), 2.88 (q, J = 7.2 Hz, 2H), 1.27 (t, J = 7.2 Hz, 3H). |

### Synthesis of 2001-B1

### 7-chloro-8-ethyl-10-(2-hydroxyethyl)-3-(4-methoxybenzyl)benzo[g]pteridine-2,4(3H, 10H)-dione (JG-2001-B1)-

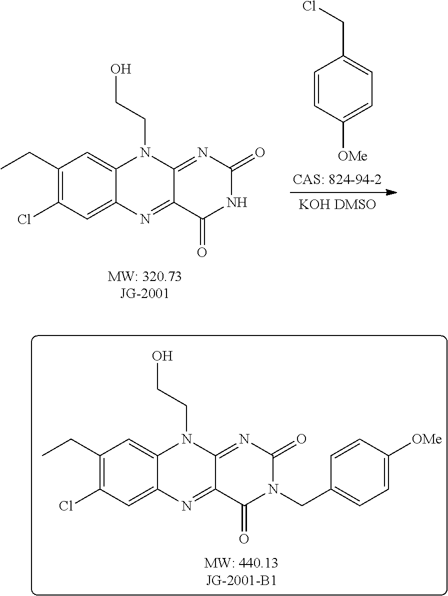 [SMILES: CCc1cc2c(cc1Cl)nc1c(=O)[nH]c(=O)nc-1n2CCO ; COc1ccc(CCl)cc1 ; CCc1cc2c(cc1Cl)nc1c(=O)n(Cc3ccc(OC)cc3)c(=O)nc-1n2CCO]

JG-2001 (100 mg, 1 eq) was mixed with CAS 824-94-2 (3 eq) and KOH (4 eq) in DMSO and reacted at room temperature for 2 hours. Product (JG-2001-B1) was purified by column chromatography (yield 25 mg). ¹H-NMR (400 MHz, DMSO, ppm) δ 8.25 (s, 1H), 8.08 (s, 1H), 7.32 (d, J=8.4 Hz, 2H), 6.86 (d, J=8.4 Hz, 2H), 5.02 (s, 2H), 4.96 (t, J=6.0 Hz, 1H), 4.73 (s, 2H), 3.82 (d, J=5.6 Hz, 2H), 3.71 (s, 3H), 2.94 (d, J=7.6 Hz, 2H), 1.29 (t, J=7.6 Hz, 3H). LCMS, calc'd 440.13 m/z; found 441.79 m/z.

### Synthesis of JG-2011

### 7-chloro-8-ethyl-10-(2-(pyrazin-2-ylmethoxy)ethyl)benzo[g]pteridine-2,4(3H, 10H)-dione (JG-2011)-

### Part-1: Amine Sidechain Synthesis

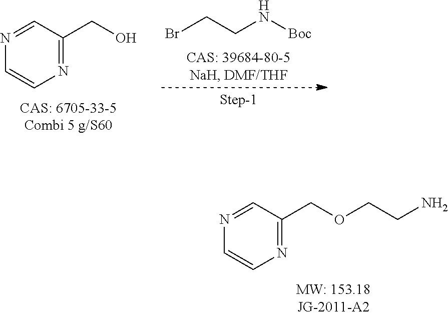 [SMILES: OCc1cnccn1 ; CC(C)(C)OC(=O)NCCBr ; NCCOCc1cnccn1]

CAS 6705-33-5 (100 mg, 1 eq) was mixed with NaH (5 eq) and CAS 39684-80-5 (2 eq) in THF, then chilled to 0° C. on ice and allowed to warm to room temperature for 3 hours. Product formation (JG-2011-A2) was confirmed by TLC and LCMS, purified by reverse phase chromatography (yield 590 mg).

Part-2: Synthesis of JG-2011 from Amine Sidechain

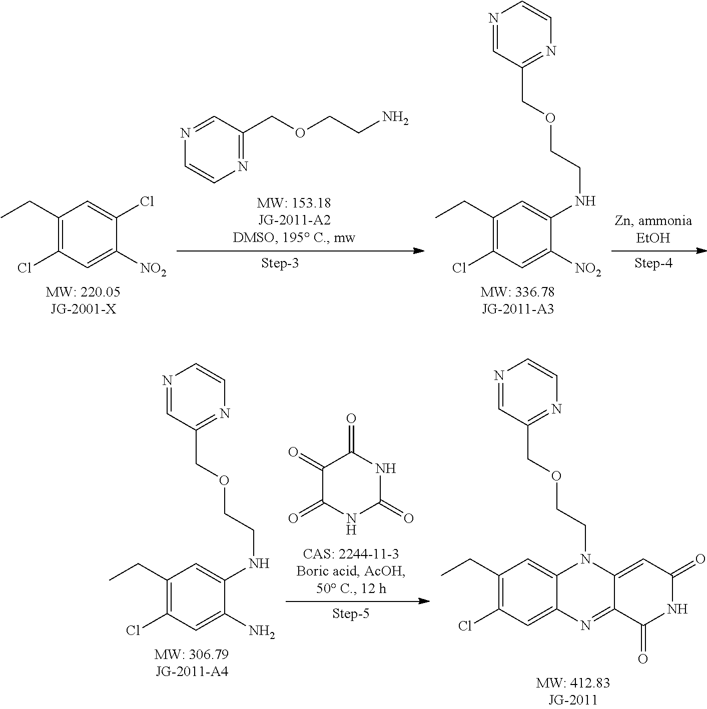

JG-2001-X (238 mg, 1 eq) was combined with JG-2011-A2 (3 eq) in DMSO, then heated to 190° C. for 15 minutes in a microwave. Product formation (JG-2011-A3) was observed by TLC and LCMS, and purified by column chromatography (yield 63 mg). Then, JG-2011-A3 (63 mg, 1 eq) was combined with Zn (8 eq) and NH4Cl (8 eq) in a 8:2 mixture of ethanol:water, reacted for 30 minutes at 80° C. and product formation (JG-2011-A4) was confirmed by TLC, LCMS, purified by column chromatography (yield 54 mg). Next, JG-2011-A4 (54 mg, 1 eq) was mixed with alloxan, boric acid and acetic acid, heated to 80° C. for 15 and product formation (JG-2011) was observed, confirmed by TLC, purified by column chromatography (yield 7 mg). 1H NMR (400 MHz, DMSO, ppm) δ 11.46 (s, 1H), 8.52 (s, 2H), 8.42 (s, 1H), 8.20 (s, 1H), 8.08 (s, 1H), 4.90 (s, 2H), 4.63 (s, 2H), 4.02 (s, 2H), 2.86 (s, 2H), 1.16 (s, 3H). LCMS, calc'd 412.83 m/z; found 413.64 m/z.

### Synthesis of JG-2031

### 2-(7-chloro-8-ethyl-2,4-dioxo-3,4-dihydrobenzo[g]pteridin-10(2H)-yl)ethyl phenylcarbamate (JG-2031)-

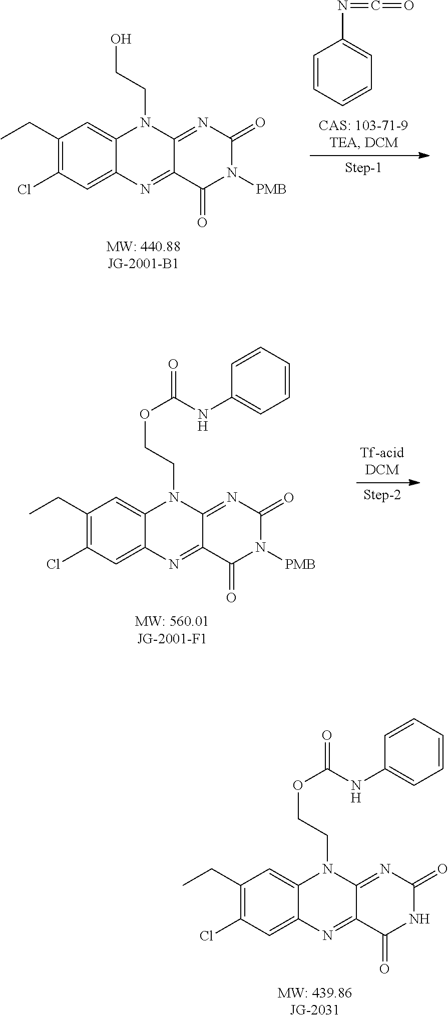

JG-2001-B1 (80, 1 eq) was mixed with DCM+TEA (3 eq) and CAS 103-71-0 (1.5 eq) for 1 hour at room temperature. Product formation (JG-2001-F1) was observed by TLC and LCMS, and purified by column chromatography (yield 50 mg). JG-2001-F1 was mixed with DCM and triflic acid (0.1 ml) and cooled on ice for 30 minutes. Product formation (JG-2031) was observed by TLC and LCMS (yield 20 mg). 1H NMR (400 MHz, DMSO, ppm) δ 11.51 (s, 1H), 9.5 (s, 1H), 8.22 (s, 1H), 7.9 (s, 1H), 7.27 (d, 4H), 6.97 (m, 1H), 4.9 (s, 2H), 4.5 (s, 2H), 2.85 (d, J=7.2 Hz, 2H), 1.21 (t, J=6.8 Hz, 3H). LCMS, calc'd 439.86 m/z; found 440.74 m/z.

### Synthesis of JG-2029

### 2-(7-chloro-8-ethyl-2,4-dioxo-3,4-dihydrobenzo[g]pteridin-10(2H)yl)ethylbenzoate (JG-2029)

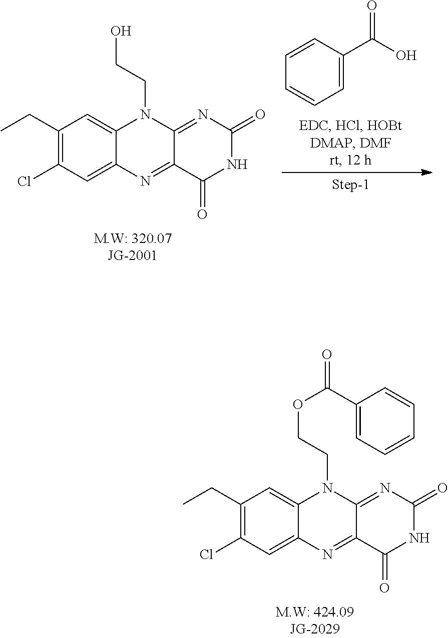

To a solution of benzoic acid (0.15 g, 0.12 mmol) in DMF (3 mL), EDC.HCl (0.35 g, 0.18 mmol), DMAP (0.45 g, 0.17 mmol) and HOBT (0.24 g, 0.17 mmol) were added. 7-chloro-8-ethyl-10-(2-hydroxyethyl)benzo[g]pteridine-2,4(3H, 10H)-dione (JG-2001) (0.39 g, 0.12 mmol) in DMF (2 mL) was added to the reaction mixture and stirred at rt for 12 min. The reaction mixture was slowly poured into water (10 mL) and extracted with ethyl acetate (3×10 mL). The combined organic layer was dried over anhydrous sodium sulphate and concentrated under vacuum. The crude was purified with prep-HPLC to obtained title compound as pale yellow solid (0.036 g, 18.18%). LCMS: 425.19 (M+1); 1H NMR (400 MHz, DMSO-d6) δ 11.49 (s, 1H), 8.25 (s, 1H), 8.22 (s, 1H), 7.73-7.59 (m, 3H), 7.43-7.39 (m, 2H), 5.06 (t, 2H), 4.71 (bs, 2H), 2.87 (q, J=7.2 Hz, 2H), 1.21 (t, J=7.2 Hz, 3H).

### Synthesis of JG-2030

### 2-(7-chloro-8-ethyl-2,4-dioxo-3,4-dihydrobenzo[g]pteridin-10(2H)-yl)ethylisobutyrate (JG-2030)

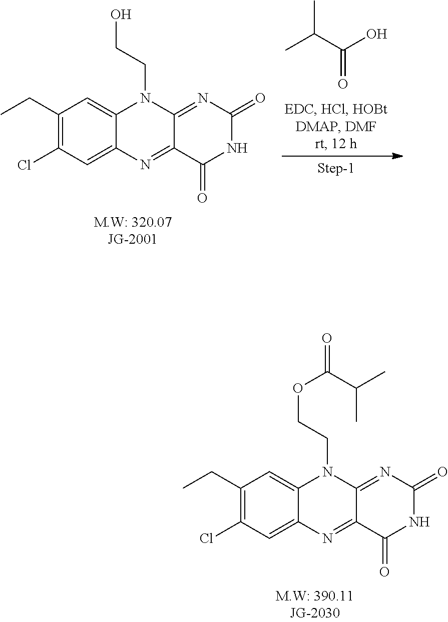

To a solution of Isobutyric acid ((0.15 g, 0.17 mmol) in DMF (3 mL), EDC.HCl (0.48 g, 0.25 mmol), DMAP (0.34 g, 0.25 mmol) and HOBT (0.34 g, 0.25 mmol) were added and stirred at room temperature. 7-chloro-8-ethyl-10-(2-hydroxyethyl)benzo[g]pteridine-2,4(3H, 10H)-dione (JG-2001) (0.54 g, 0.16 mmol) in DMF (3 mL) was added and stirred for 10 min ar room temperature. The reaction mixture was slowly poured into water (10 mL) and extracted with ethyl acetate (3×10 mL). The combined organic layer was dried over anhydrous sodium sulphate and concentrated under vacuum. The crude was purified by prep-HPLC to obtained title compound as pale yellow solid (0.066 g, 36.76%). LCMS: 391.8, 393.8 (M& M+2); 1H NMR (400 MHz, DMSO-d6) δ 11.49 (s, 1H), 8.23 (s, 1H), 8.08 (s, 1H), 4.93 (t, 2H), 4.44 (s, 2H), 2.95 (q, J=7.6 Hz, 2H), 2.36 (hept, 1H), 1.31 (t, J=7.6 Hz, 3H), 0.90 (d, J=6.8 Hz, 6H).

### Synthesis of JG-2066

### 2-(7-chloro-8-ethyl-2,4-dioxo-3,4-dihydrobenzo[g]pteridin-10(2H)-yl)ethylethylcarbamate (JG-2066)-

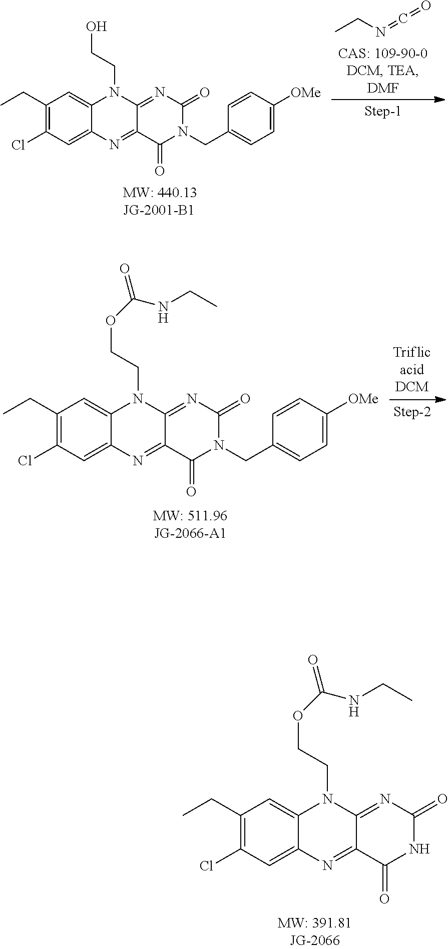

JG-2001-B1 (30 mg, 1 eq) was mixed with CAS 109-90-4 (4 eq), DCM, TEA and DMR (0.2 ml) for 2 hours at room temperature. Product (JG-2066-A1) was observed by TLC (yield 20 mg). The product was mixed with ice cold DCM and triflic acid for 1 hour at 00 C. Product (JG-2066) was observed by TLC and purified by column chromatography (yield 13 mg). 1H NMR (400 MHz, DMSO, ppm) δ 11.49 (s, 1H), 8.33 (s, 1H), 7.95 (s, 1H), 7.11 (t, J=5.6 Hz, 1H), 4.85 (t, 2H), 4.36 (t, J=5.2 Hz, 3H), 2.90 (m, 4H), 1.31 (t, J=7.6 Hz, 3H), 0.85 (t, J=7.2 Hz, 3H). LCMS, calc'd 391.81 m/z; found 392.07 m/z.

### Synthesis of JG-2016 (Scale-Up)

### 7-chloro-8-ethyl-10-(2-isobutoxyethyl)benzo[g]pteridine-2,4(3H, 10H)-dione

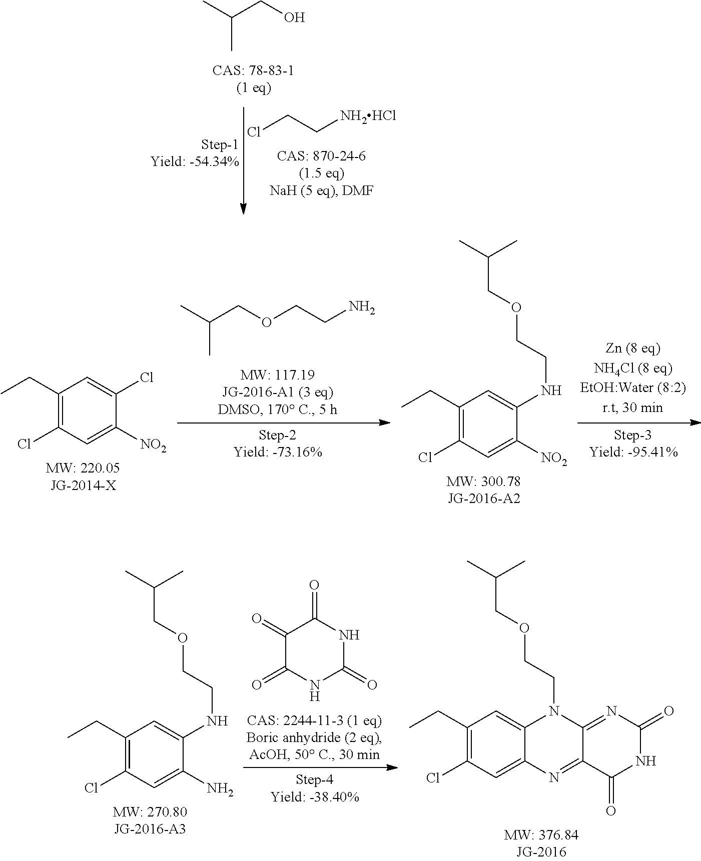

### Step-1 (JG-2016-A1)

To a solution of 2-methylpropan-1-ol (CAS: 78-83-1) (25 g, 337.2 mmol, 1 eq) in DMF (250 mL/10V), 60% NaH (67.45 g, 1686.4 mmol, 5.0 eq) was added portion wise and stirred at 0° C. temperature for 1 h under nitrogen atmosphere. 2-chloroethan-1-amine hydrochloride (CAS: 870-24-6) (58.68 g, 505.9 mmol, 1.5 eq) was added portion wise to the reaction mixture and stirred at room temperature for 4 h. After completion of reaction as indicated by TLC, the reaction mixture was poured into aqueous sodium chloride solution and extracted with ethyl acetate (3×2 L). The combined organic layer was dried over anhydrous sodium sulphate and concentrated under vacuum up to 300 ml then washed with water (2×500 mL). The organic layer was dried over anhydrous sodium sulphate and concentrated under vacuum to afford yellow biphasic liquid (21.48 g, 54.34%) crude material which was directly used in next step without further purification. LCMS: 118.2 (M+1).

### Step-2 (JG-2016-A2)

To a solution of 1,4-dichloro-2-ethyl-5-nitrobenzene (JG-2014-X) (5.0 g, 22.72 mmol, 1.0 eq) in DMSO (5 mL), JG-2016-A1 (13.31 g, 113.6 mmol, 5.0 eq) was added and stirred at 170° C. temperature for 5 h. After completion of reaction as indicated by TLC, the reaction mixture was poured into ice cooled water (300 mL) and extracted with ethyl acetate (3×100 mL). The combined organic layer was dried over anhydrous sodium sulphate and concentrated under vacuum. The crude was further purified by column chromatography (4% ethyl acetate/hexane) to afford yellow liquid (5.0 g, 73.16%) LCMS: 301.3, 303.3 (M & M+2).

### Step-3 (JG-2016-A3)

To a solution of 4-chloro-5-ethyl-N-(2-isobutoxyethyl)-2-nitroaniline (JG-2016-A2) (6.1 g, 20.28 mmol, 1.0 eq) in ethanol:water (8:2V) Zn Dust (10.60 g, 162.2 mmol, 8.0 eq) and NH4Cl (8.67 g, 162.2 mmol, 8.0 eq) were added and stirred the reaction mixture at room temperature for 30 min at room temperature. TLC indicated completion of reaction. The reaction mixture was frittered through celite and filtrate was extracted with ethyl acetate (2×50 mL). The organic layer was dried over anhydrous sodium sulphate and concentrated under reduce pressure to afford yellow liquid (5.24 g, 95.41%). The crude product was used in next step without further purification. LCMS: 271.3, 273.2 (M&M+2).

### Step-4 (JG-2016)

To a solution of 4-chloro-5-ethyl-N₁-(2-isobutoxyethyl)benzene-1,2-diamine (JG-2016-A3) (5.24 g, 19.35 mmol, 1.0 eq) in AcOH (25 mL) alloxan monohydrate (CAS: 2244-11-3) (3.09 g, 19.35 mmol, 1.0 eq) and boric anhydride (2.69 g, 38.70 mmol, 2.0 eq) were added and stirred at 50° C. temperature for 30 min. The reaction mixture was poured into ice cooled water (100 mL) and extracted with ethyl acetate (3×50 mL). The reaction mixture was dried over anhydrous sodium sulphate Filtrate was concentrated under reduce pressure. The obtained crude product was further purified by column chromatography (80% ethyl acetate/hexane) and pure fraction was concentrated under vacuum to afford yellow solid (2.8 g, 38.40%). LCMS: 377.3, 379.3 (M&M+2). 1H NMR (400 MHz, DMSO) δ 11.44 (s, 1H), 8.18 (s, 1H), 8.03 (s, 1H), 4.80 (t, 2H), 3.78 (t, J=4.8 Hz, 2H), 3.12 (d, J=6.4 Hz, 2H), 2.91 (q, J=7.3 Hz, 2H), 1.62 (m, 1H), 1.27 (t, J=7.4 Hz, 3H), 0.70 (d, J=6.7 Hz, 6H).

1. A compound of formula 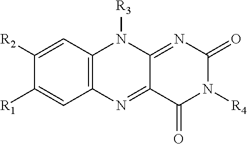
or its salt of a suitable acid,
wherein:
R₁ is a functional group selected from H, OH, methyl (CH₃), ethyl (C₂H₅), ethyleny (C₂H₃), ethynyl (C₂H), halogen, alkyl, alkoxy, azide (Ns), ether, NO₂, or cyanide (CN);
R₂ is a functional group selected from H, OH, methyl (CH₃), ethyl (C₂H₅), ethyleny (C₂H₃), ethynyl (C₂H), halogen, alkyl, alkoxy, azide (Ns), ether, NO₂, or cyanide (CN);
R₃ is a functional group selected from H, glucose, ribose, an alkyl chain, an alkoxy chain, a ribityl chain, OH, (CH₂)ₙOC═OR₁′, (CH₂)ₙNR₂′R₃′, (CH₂)ₙOH, (CHOH)ₙ-alkyl, (CHOH)ₙ-alkyne, (CHOH)ₙ-aryl, (CH₂)ₙOR₁′, C(OC═OR₁′)ₙ, where R₁′ is an alkyl, aryl and R₂′ and R₃′ are each independently an alkyl, alkene, alkyne, aryl, (CH₂)ₙOH, CHOH-alkyl, CHOH-alkyne, or CHOH-aryl;
R₄ is a functional group selected from H, OH, methyl, (CH₃), ethyl (C₂H₆), halogen, alkyl (including cyclic alkyls), alkoxy, azide (Ns), ether, NO₂, cyanide (CN), benzyl, and (CH₂)ₙ-benzyl; and
n is an independently selected integer selected from 1, 2, 3, or 4.

2. The compound of formula of claim 1, wherein R₁ is a C₁ to C₃ alkyl and R₂ is a halogen.

3. The compound of formula of claim 1, wherein R₁ is ethyl (C₂H₆) or methyl (CH₃) and R₂ is F, Cl, or Br.

4. The compound of formula of claim 1, wherein R₁ is ethyl (C₂H₆) and R₂ is Cl.

5. The compound of formula of claim 1, wherein R₂ is a C₁ to C₃ alkyl and R₁ is a halogen.

6. The compound of formula of claim 1, wherein R₂ is ethyl (C₂H₆) or methyl (CH₃) and R₁ is F, Cl, or Br.

7. The compound of formula of claim 1, wherein R₂ is ethyl (C₂H₆) and R₁ is Cl.

8. The compound of formula of claim 1, wherein R₁ and R₂ are each independently a C₁ to C₃ alkyl.

9. The compound of formula of claim 1, wherein R₁ and R₂ are each independently ethyl (C₂H₆) or methyl (CH₃).

10. The compound of formula of claim 1, wherein R₁ and R₂ are each independently ethyl (C₂H₆).

11. The compound of formula of claim 1, wherein R₁ and R₂ are each independently methyl (CH₃).

12. The compound of formula of claim 1, wherein R₁ and R₂ are each independently a halogen.

13. The compound of formula of claim 1, wherein R₁ and R₂ are each independently F, Cl, or Br.

14. The compound of formula of claim 1, wherein R₁ and R₂ are each independently C₁.

15. The compound of formula of claim 1, wherein R₃ is ribose, an alkyl chain, an alkoxy chain, or a ribityl chain.

16. The compound of formula of claim 1, wherein R₃ is ribose,

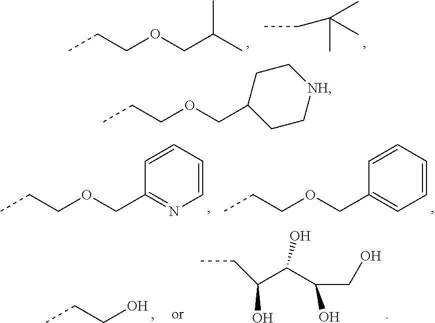

17-70. (canceled)

71. A method of treatment of a mammalian disorder or condition comprising: administering a pharmaceutical formulation to a mammalian subject, the pharmaceutical formulation containing a therapeutically effective amount of one or more small molecule compounds having the formula 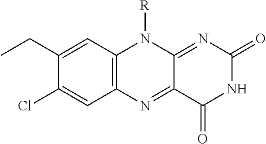
or an acceptable pharmaceutical salt thereof,
wherein:
R is a functional group selected from H, glucose, ribose, an alkyl chain, an alkoxy chain, a ribityl chain, OH, (CH₂)ₙOC═OR₁′, (CH₂)ₙNR₂′R₃′, (CH₂)ₙOH, (CHOH)ₙ-alkyl, (CHOH)ₙ-alkyne, (CHOH)ₙ-aryl, (CH₂)ₙOR₁′, C(OC═OR₁′)ₙ, where R₁′ is an alkyl, alkene, alkyne, aryl and R₂′ and R₃′ are each independently an alkyl, alkene, alkyne, aryl, (CH₂)ₙOH, CHOH-alkyl, CHOH-alkyne, or CHOH-aryl; and
n is an independently selected integer selected from 1, 2, 3, or 4.

72. (canceled)

73. The method of claim 71, wherein the mammalian disorder or condition is a neoplasm, and wherein the neoplasm is selected from one or more of the group: acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, neuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors.

74-94. (canceled)

95. The compound of claim 1, wherein the compound is JG-2016.

96. The method of claim 71, wherein the one or more small molecule compounds comprises JG-2016.
